SEQUENCE LISTING (11 sequences)

SEQ ID NO: 1 - protein, 9 aa, Artificial
  Peptide corresponding to HIV gag
AMQMLKETI

SEQ ID NO: 2 - protein, 9 aa, Artificial
  Intracellular degradation motif
RXALGXIXN

SEQ ID NO: 3 - protein, 10 aa, Artificial
  Intracellular degradation motif
KTKRNYSARD

SEQ ID NO: 4 - DNA, 21 bp, Artificial
  Fw1 primer
atgaagatctctgcagctgcc

SEQ ID NO: 5 - DNA, 21 bp, Artificial
  REV1 primer
ctagctcatctccaaatagtt

SEQ ID NO: 6 - DNA, 21 bp, Artificial
  Fw2 primer
tcaccatcatcctcactgcag

SEQ ID NO: 7 - DNA, 21 bp, Artificial
  REV2 primer
tcgagtgacaaacacgactgc

SEQ ID NO: 8 - DNA, 38 bp, Artificial
  IL-13Ralpha2 - Forward primer
agatctgaaatggcttttgtgcatatcagatgcttgtg

SEQ ID NO: 9 - DNA, 30 bp, Artificial
  IL-13Ralpha2 - Reverse primer
gagctcttaacagagggtatcttcataagc

SEQ ID NO: 10 - protein, 5 aa, Homo sapiens
ARINV

SEQ ID NO: 11 - protein, 11 aa, Escherichia coli
AANDENYALAA

(19) United States
(12) Patent Application Publication
(10) Pub. No.: US 2012/0177668 A1
(43) Pub. Date: Jul. 12, 2012

(54) IMMUNOMODULATING COMPOSITIONS COMPRISING INTERLEUKIN 13 INHIBITORS AND USES THEREFOR

(75) Inventors: Ronald James Jackson, Mckellar (AU); Ian Allister Ramshaw, Duffy (AU); Charani Ranasinghe, Aranda (AU)

(73) Assignee: THE AUSTRALIAN NATIONAL UNIVERSITY, Australian Capital Territory (AU)

(21) Appl. No.: 13/381,757

(22) PCT Filed: Jul. 6, 2010
(86) PCT No.: PCT/AU10/00864

§ 371 Date: Mar. 20, 2012

(30) Foreign Application Priority Data
Jul. 6, 2009 (AU) 2009903156

(51) Int. Cl. A61K 39/00; A61P 37/02

(52) U.S. Cl. 424/184.1

(57) ABSTRACT

This invention relates generally to compositions and methods for modulating immune responses. More particularly, the present invention relates to the co-expression, co-location or co-presentation on host cells (e.g. antigen-presenting cells, leukocytes, etc) of an inhibitor of IL-13 function and an immune stimulator that stimulates an immune response to a target antigen in compositions and methods for stimulating protective or therapeutic immune responses to the target antigen. The compositions and methods of the present invention are particularly useful in the prophylaxis and/or treatment of a range of diseases or conditions including pathogenic infections and cancers.

### FIELD OF THE INVENTION

This invention relates generally to compositions and methods for modulating immune responses. More particularly, the present invention relates to the co-expression, co-location or co-presentation on host cells (e.g. antigen-presenting cells, leukocytes, etc) of an inhibitor of IL-13 function and an immune stimulator that stimulates an immune response to a target antigen in compositions and methods for stimulating protective or therapeutic immune responses to the target antigen. The compositions and methods of the present invention are particularly useful in the prophylaxis and/or treatment of a range of diseases or conditions including pathogenic infections and cancers.

### BACKGROUND OF THE INVENTION

Remarkable strides have been made in the last two centuries in the control of human infectious diseases through the development of continuously improved vaccination techniques. Indeed, past vaccine development has proved to be the single most cost effective investment in health care.

The immune system protects us against infection through the induction of either neutralising antibodies or cell-mediated immunity (CMI). Infectious pathogens that can be neutralised by antibody such as polio, influenza, hepatitis B, and HPV have well-established classical routes to vaccine development. However, antibodies may not be effective at blocking many infections such as human immunodeficiency virus (HIV), tuberculosis (TB), non-pharyngeal carcinoma and hepatitis C, thus vaccine strategies that stimulate good CMI responses are required to combat these infections. Unfortunately, vaccines that stimulate strong, protective CMI responses have proven far more difficult to develop.

Many of the current HIV vaccine trials, for example, although showing enhanced immunity in animals, have failed to establish true correlates of protection. These findings increasingly suggest that not only the magnitude but also the “quality” or “avidity” of the T cell response generated against vaccine antigens may be important in protection against pathogenic organisms such as HIV-1. The quality of the T cell response is reflected in the functional avidity of T cells towards the MHC-peptide complex on target cells. High avidity cytotoxic T lymphocytes (CTL) recognise low concentrations of antigen, whilst low avidity CTL are functionally ineffective at these antigen concentrations (Alexander-Miller et al., 1996, J. Exp. Med. 184: 485-492; La Gruta et al., 2004, J. Immunol. 172: 5553-5560). It is now well established that high avidity CTL also have greater capacity to clear an infection compared to low avidity T cells (Alexander-Miller et al., 2005, Immunol. Res. 31: 13-24).

In work leading up to the present invention, it was discovered that expression of the cytokine IL-13 plays an important role in down-regulating the functional avidity of cytotoxic T-cells, and that T-cell avidity is thus improved by inhibition of IL-13 function in the local milieu of the immune response. These discoveries led the inventors to also discover that a subject's T-cell mediated immune response to a target antigen may be enhanced by inhibition of IL-13 function in the local milieu of the immune response.

The above discoveries have been reduced to practice in novel compositions and methods and compositions for stimulating more efficacious prophylactic and therapeutic immune responses against a target antigen, including those associated with a disease or condition, including pathogenic infections and cancers.

### SUMMARY OF THE INVENTION

Accordingly, in one aspect, the present invention provides compositions for stimulating an immune response against a target antigen in a subject. In certain embodiments, the immune response is a T-cell mediated response. In another aspect, the present invention provides compositions for preventing or treating a disease or condition associated with the presence or aberrant expression of a target antigen in a subject.

The compositions of the present invention generally comprise a first agent comprising an immune stimulator that stimulates or otherwise enhances an immune response to the target antigen or a polynucleotide sequence encoding an immune stimulator that stimulates or otherwise enhances an immune response to the target antigen together with a second agent comprising an inhibitor of IL-13 function or a polynucleotide sequence encoding an inhibitor of IL-13 function.

In some embodiments, the composition comprises a nucleic acid composition comprising: a first agent comprising a first polynucleotide sequence which encodes an immune stimulator that stimulates or otherwise enhances an immune response to the target antigen and which is operably linked to a regulatory polynucleotide, and a second agent comprising a second polynucleotide sequence which encodes an inhibitor of IL-13 function and which is operably linked to a regulatory polynucleotide.

In some embodiments, the immune stimulator is selected from an antigen that corresponds to at least a portion of the target antigen. The target antigen is typically associated with a disease or condition of interest, including but not limited to pathogenic infections and cancers, such as but not limited to HIV, TB, non-pharyngeal carcinoma and hepatitis C. The antigen that corresponds to at least a portion of the target antigen may be in soluble form (e.g., a peptide or polypeptide) when expressed.

In some embodiments, the inhibitor of IL-13 function is selected from a modified, mutated or defective form of IL-13, soluble or defective IL-13 receptors or fragments thereof, or antigen-binding molecules that are immuno-interactive with IL-13 or an IL-13 receptor.

In some embodiments, the composition further comprises a third agent comprising an inhibitor of IL-4 function or a polynucleotide sequence encoding an inhibitor of IL-4 function. In some embodiments, the inhibitor of IL-4 function is selected from a mutated or defective form of IL-4, soluble or defective IL-4 receptors or fragments thereof, or antigen-binding molecules that are immuno-interactive with IL-4 or an IL-4 receptor.

In some embodiments, the subject is naïve to the target antigen or has previously raised an immune response to the target antigen. Suitably, in embodiments in which the subject has previously raised an immune response to the target antigen and the immune stimulator comprises an antigen that corresponds to the target antigen, the amino acid sequence of the corresponding antigen is the same as the amino acid sequence of at least a portion of the target antigen. In illustrative examples of this type, the corresponding antigen is a naturally-occurring antigen to which the subject has previously raised an immune response.

Exemplary compositions of the present invention include vaccines or constructs, including but not limited to recombinant vaccines.

In some embodiments, the compositions further comprise a pharmaceutically acceptable carrier or diluent. In some embodiments, the compositions further comprise an adjuvant that enhances the effectiveness of the immune stimulation. Suitably, the adjuvant delivers the antigen to the class I major histocompatibility (MHC) pathway. For example, such adjuvants include, but are not limited to, saponin-containing compounds (e.g., ISCOMs) and cytolysins, which mediates delivery of antigens to the cytosol of a target cell. The cytolysin may be linked to, or otherwise associated with, the antigen. In some embodiments, the cytolysin mediates transfer of the antigens from the vacuole (e.g., phagosome or endosome) to the cytosol of an antigen-presenting cell and in illustrative examples of this type, the cytolysin is a listeriolysin.

In some embodiments, the antigen comprises, or is otherwise associated with, an intracellular degradation signal or degron. In illustrative examples of this type, the intracellular degradation signal comprises a destabilising amino acid at the amino-terminus of the antigen. Suitably, the destabilising amino acid is selected from isoleucine and glutamic acid, preferably from histidine tyrosine and glutamine, and even more preferably from aspartic acid, asparagine, phenylalanine, leucine, tryptophan and lysine. In a specific embodiment, the destabilising amino acid is arginine. In other illustrative examples of this type, the antigen is fused or otherwise conjugated to a masking entity, which masks the amino terminus so that when unmasked the antigen will exhibit an enhanced rate of intracellular proteolytic degradation. Suitably, the masking entity is a masking protein sequence. The masking protein sequence is suitably cleavable by an endoprotease, which is typically an endogenous endoprotease of a mammalian cell. For example, an endoprotease cleavage site may be interposed between the masking protein sequence and the antigen. Suitable endoproteases include, but are not restricted to, serine endoproteases (e.g., subtilisins and furins), proteasomal endopeptidases, proteases relating to the MHC class I processing pathway and signal peptidases. In a preferred embodiment of this type, the masking protein sequence comprises a signal peptide sequence. Suitable signal peptides sequences are described, for example, by Nothwehr et al. (1990, Bioessays 12 (10): 479-484), Izard, et al. (1994, Mol. Microbiol. 13 (5): 765-773), Menne, et al. (2000, Bioinformatics. 16 (8): 741-742) and Ladunga (2000, Curr. Opin. Biotechnol. 11 (1): 13-18).

Alternatively or in addition, the intracellular degradation signal comprises an ubiquitin acceptor, which allows for the attachment of ubiquitin by intracellular enzymes, which target the antigen for degradation via the ubiquitin-proteosome pathway. Suitably, the ubiquitin acceptor is a molecule which contains a residue appropriately positioned from the amino terminus of the antigen as to be able to be bound by ubiquitin molecules. Such residues may have an epsilon amino group such as lysine. In illustrative examples of this type, the ubiquitin acceptor comprises at least one, preferably at least two, more preferably at least four and still more preferably at least six lysine residues, which are suitably present in a sufficiently segmentally mobile region of the antigen.

In some embodiments, the intracellular degradation signal comprises a ubiquitin or biologically active fragment thereof. In non-limiting examples of this type, the ubiquitin or biologically active fragment thereof is fused, or otherwise conjugated, to the antigen. Suitably, the ubiquitin is of mammalian origin, more preferably of human or other primate origin.

Another aspect of the present invention provides methods for stimulating an immune response to a target antigen in a subject. In certain embodiments, the immune response is a T-cell mediated response. A further aspect of the present invention provides methods for treating or preventing a disease or condition associated with the presence or aberrant expression of a target antigen in a subject.

The methods of the present invention generally comprise administering to the subject an effective amount of a first agent comprising an immune stimulator that stimulates or otherwise enhances an immune response to the target antigen or a polynucleotide sequence encoding an immune stimulator that stimulates or otherwise enhances an immune response to the target antigen together with a second agent comprising an inhibitor of IL-13 function or a polynucleotide sequence encoding an inhibitor of IL-13 function, as broadly described above. The method may further comprise administering the first agent and the second agent together with a third agent comprising an inhibitor of IL-4 function or a polynucleotide sequence encoding an inhibitor of IL-4 function, as broadly described above. The target antigen is typically associated with a disease or condition of interest, including but not limited to pathogenic infections and cancers, such as but not limited to HIV, TB, non-pharyngeal carcinoma and hepatitis C.

In some embodiments, the subject is naïve to the target antigen or has previously raised an immune response to the target antigen. Suitably, in embodiments in which the subject has previously raised an immune response to the target antigen and the immune stimulator comprises an antigen that corresponds to the target antigen, the amino acid sequence of corresponding antigen is the same as the amino acid sequence of at least a portion of the target antigen. In illustrative examples of this type, the corresponding antigen is a naturally-occurring antigen to which the subject has previously raised an immune response.

In yet another aspect, the invention contemplates the use of a first agent comprising an immune stimulator that stimulates or otherwise enhances an immune response to the target antigen or a polynucleotide sequence encoding an immune stimulator that stimulates or otherwise enhances an immune response to the target antigen and a second agent comprising an inhibitor of IL-13 function or a polynucleotide sequence encoding an inhibitor of IL-13 function as broadly defined above in the manufacture of a medicament for stimulating an immune response to the target antigen in a subject. The use may further comprise the use of a third agent comprising an inhibitor of IL-4 function or a polynucleotide sequence encoding an inhibitor of IL-4 function, as broadly described above, in the manufacture of the medicament. In certain embodiments, the immune response is a T-cell mediated response. The target antigen is typically associated with a disease or condition of interest, including but not limited to pathogenic infections and cancers, such as but not limited to HIV, TB, non-pharyngeal carcinoma and hepatitis C.

In still another aspect, the invention resides in the use of a first agent comprising an immune stimulator that stimulates or otherwise enhances an immune response to the target antigen or a polynucleotide sequence encoding an immune stimulator that stimulates or otherwise enhances an immune response to the target antigen and a second agent comprising an inhibitor of IL-13 function or a polynucleotide sequence encoding an inhibitor of IL-13 function as broadly defined above in the manufacture of a medicament for preventing or treating a disease or condition associated with the presence or aberrant expression of the target antigen in a subject. The use may further comprise the use of a third agent comprising an inhibitor of IL-4 function or a polynucleotide sequence encoding an inhibitor of IL-4 function, as broadly described above, in the manufacture of the medicament. The target antigen is typically associated with a disease or condition of interest, including but not limited to pathogenic infections and cancers, such as but not limited to HIV, TB, non-pharyngeal carcinoma and hepatitis C.

Yet another aspect of the present invention provides an immunomodulatory antigen-presenting cell or antigen-presenting cell precursor that presents an antigen that corresponds to at least a portion of the target antigen, and wherein the antigen-presenting cell or antigen-presenting cell precursor expresses or otherwise produces an inhibitor of IL-13 function. In some embodiments, the antigen-presenting cell or antigen-presenting cell precursor expresses or otherwise produces an inhibitor of IL-4 function.

Yet a further aspect of the present invention provides a method for producing an immunomodulatory antigen-presenting cell, the method comprising contacting an antigen-presenting cell or antigen-presenting cell precursor with an antigen that corresponds to at least a portion of the target antigen or a composition of the invention for a time and under conditions sufficient for the antigen or a processed form thereof to be presented by the antigen-presenting cell or antigen-presenting cell precursor, and wherein the antigen-presenting cell or antigen-presenting cell precursor expresses or otherwise produces an inhibitor of IL-13 function. In some embodiments, the antigen-presenting cell or antigen-presenting cell precursor expresses or otherwise produces an inhibitor of IL-4 function.

### BRIEF DESCRIPTION OF THE DRAWINGS

FIGS. 1-7 show the results of studies described in Example 1.

FIG. 1 shows the results of studies examining K^{d}Gag₁₉₇₋₂₀₅-specific T-cell avidity, IFN-γ expression and IL-4 and IL-13 expression following mucosal and systemic immunisation as described in the Experimental section

FIG. 1A is a graphical representation showing percentage of K^{d}Gag₁₉₇₋₂₀₅ CD8⁺ splenocyte loss (dissociation) in splenocytes taken 14 days post AE VV boost from wild type BALB/c (H-2^{d}) mice (n=4 per group) that were immunized i.n./i.n. (grey line with triangles), i.n./i.m. (black dotted linen with squares), or i.m./i.m. (black line with circles) with AE FPV/AE VV as described in the Materials and Methods section. Data are values obtained using splenocytes pooled from the mice within each group and are representative of at least three experiments (this data was also reported in Ranasinghe et al., 2007, J. Immunol., 178: 2370-2379).

FIG. 1B shows plots of the results where K^{d}Gag₁₉₇₋₂₀₅-specific CTL isolated from the mice immunised by different routes were sorted, cultured in complete RPMI in the presence of IL-2 for 3-4 days, re-stimulated with AMQMLKETI gag peptide for 6 hours (after adding spleen cells from naïve BALB/c mice) and flow cytometric analysis performed as described in the Materials and Methods section to evaluate the proportion of IFN-γ⁺ HIV-specific CTL expressed as a percentage of the total number of antigen-specific CTL. The plots represent the i.n./i.n. immunised unstimulated control (left, note that the i.m./i.m. immunised unstimulated control gave comparable results (data not shown)), i.n./i.n. immunised peptide-stimulated group (middle) and i.m./i.m. immunised peptide-stimulated group (right). The y-axis indicates the IFN-γ FITC channel and the x-axis the CD8α APC channel. Data are values obtained using splenocytes pooled from the mice within each group.

FIG. 1C shows IL-4 expression by K^{d}Gag₁₉₇₋₂₀₅-specific memory CTL. Eleven months following poxvirus AE FPV/AE VV, i.m./i.m. (right) and i.n./i.m. (left) prime boost immunisation, mice (n=3-4) were challenged i.r. with AE-VV. Three days following challenge spleen cells were harvested and stimulated overnight with the AMQMLKETI gag peptide as described in the Materials and Methods section. The flow cytometry plots represent CD8⁺ T cells (gated on CD8⁺ T cells) expressing IL-4. The values in the upper right quadrants indicate the percentage of CD8⁺ T cells producing IL-4 protein. Data are values obtained using splenocytes pooled from the mice within each group.

FIG. 1D shows IL-4 and IL-13 expression by K^{d}Gag₁₉₇₋₂₀₅-specific CD8 T cells evaluated by cytokine antibody arrays. 2 weeks following i.n./i.n. or i.m./i.m. prime boost immunisation, 2×10⁶ T cells from pooled spleens (n=4 per group) were cultured overnight in complete RPMI (without any IL-2) in the presence of AMQMLKETI gag peptide. Supernatants were then collected and assessed for cytokine production as described in the Materials and Methods section. The data are representative of two experiments.

FIG. 2 shows the results of effector CTL responses in IL-4Rα^{-/-} KO and wild type BALB/c mice.

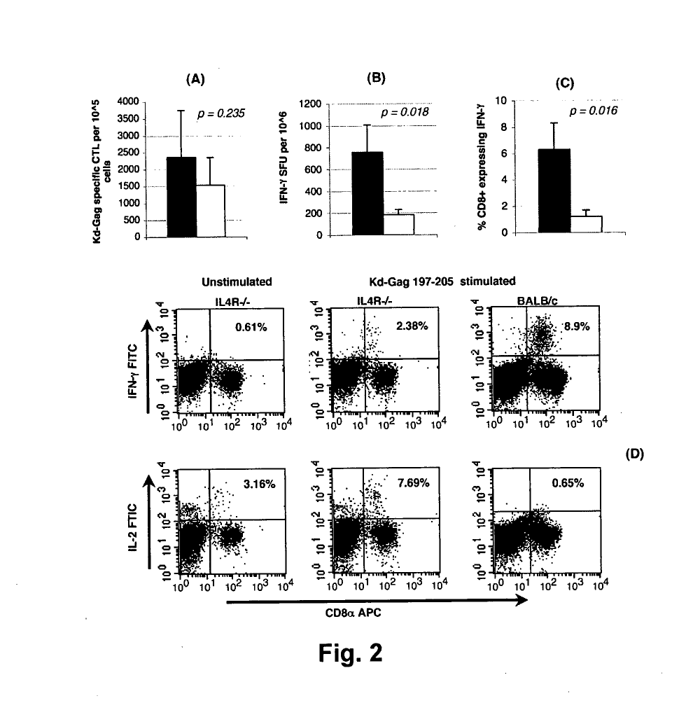

Mice (n=4-6 per group) were prime-boost immunised i.n./i.m. with AE FPV/AE VV and then K^{d}Gag₁₉₇₋₂₀₅-specific T-cell responses were measured at 14 days by tetramer staining (results shown in FIG. 2A), IFN-γ ELISpot (results shown in FIG. 2B) and IFN-γ ICS (results shown in FIG. 2C). The black bars represent the wild type BALB/c mice and the white bars represent the IL-4Rα^{-/-} KO mice. For IFN-γ ELISpot and ICS assays the splenocytes were stimulated with AMQMLKETI gag peptide; unstimulated cells from each sample were used as the background control and this value was subtracted from each sample. The data represent mean+SD and p values were determined using two-tailed, two-sample equal or unequal variance Student's t-test. The data are representative of at least three experiments.

FIG. 2D shows IFN-γ and IL-2 expression by IL-4Rα^{-/-}K^{d}Gag₁₉₇₋₂₀₅-specific and wild type BALB/cK^{d}Gag₁₉₇₋₂₀₅-specific CTL. The representative flow cytometry plots indicate CD8⁺ T cells expressing IFN-γ (top row) and IL-2 (bottom row) from an individual mouse within each group (n=4), and the percentage of CD8⁺ T cells producing either IFN-γ or IL-2 is indicated in the upper right quadrant. Un-stimulated splenocytes obtained from wild type BALB/c mice showed 0.3-0.5% background expression of IFN-γ and IL-2. The y-axis indicates the IFN-γ or IL-2 FITC channel and the x-axis the CD8α-APC channel.

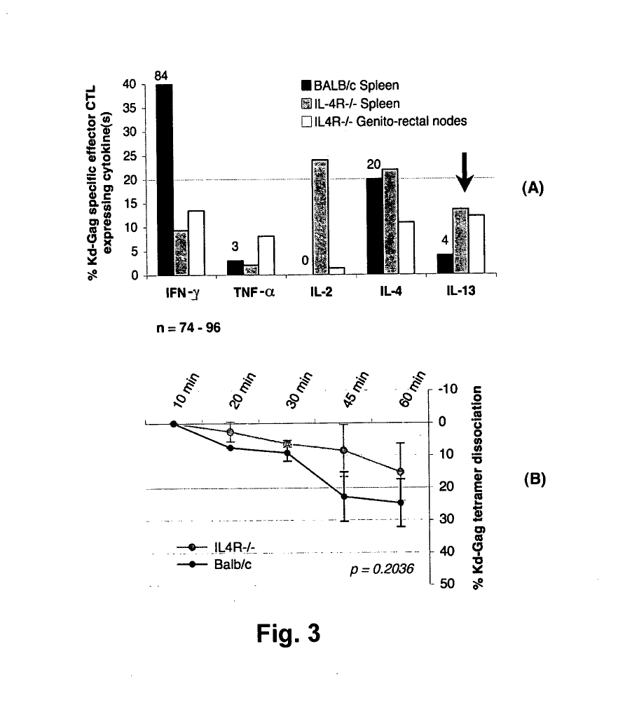

FIG. 3A shows the results of single-cell cytokine analysis of IL-4Rα^{-/-}K^{d}Gag₁₉₇₋₂₀₅-specific and wild type BALB/cK^{d}Gag₁₉₇₋₂₀₅-specific effector CTL. Mice (n=4 per group) were immunised i.n./i.m. with AE FPV/AE VV. At 14 days post boost K^{d}Gag⁺₁₉₇₋₂₀₅ single spleen (n=96) and genito-rectal node (n=74) cells were assessed for their ability to produce the indicated cytokines by single-cell multiplex nested PCR as described in the Materials and methods section. Data represent the percentage of splenocytes or genito-rectal lymphocytes producing IFN-γ, TNF-α, IL-2, IL-14 and IL-13 cytokines. The arrow highlights the differences in IL-13 expression between groups. The data are representative of two experiments. In FIG. 3B, K^{d}Gag₁₉₇₋₂₀₅-specific CTL avidity in IL-4Rα^{-/-} and wild type BALB/c mice were compared. Mice (n=4) were i.n./i.m. immunised with AE FPV/AE VV and 14 days post AE VV boost, the percentage of K^{d}Gag₁₉₇₋₂₀₅ positive CR8⁺ splenocyte loss (dissociation) was measured in individual mice (n=4) as described in the Materials and methods section. The grey line indicates IL-4Rα^{-/-} CTL and black line indicates wild type BALB/c CTL. The data represent mean±SD of four mice and p values were determined using two-tailed, two-sample equal variance Student's t-test at the 60 minutes end time point. The data are representative of at least three experiments.

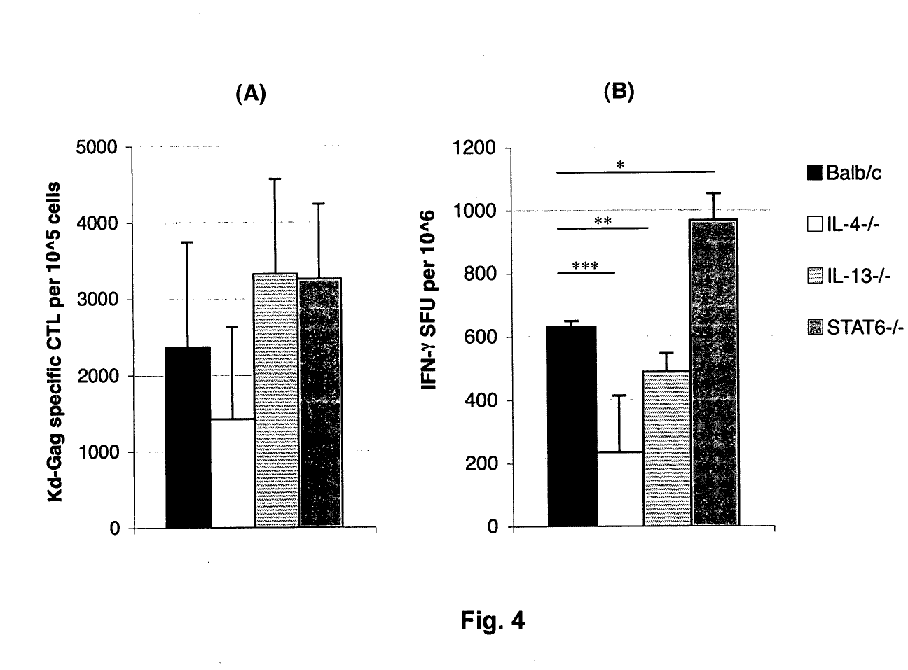

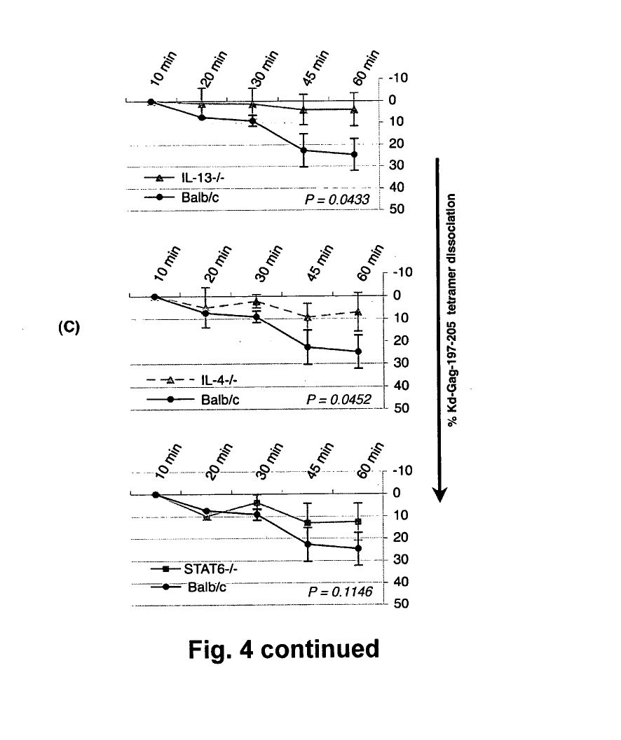

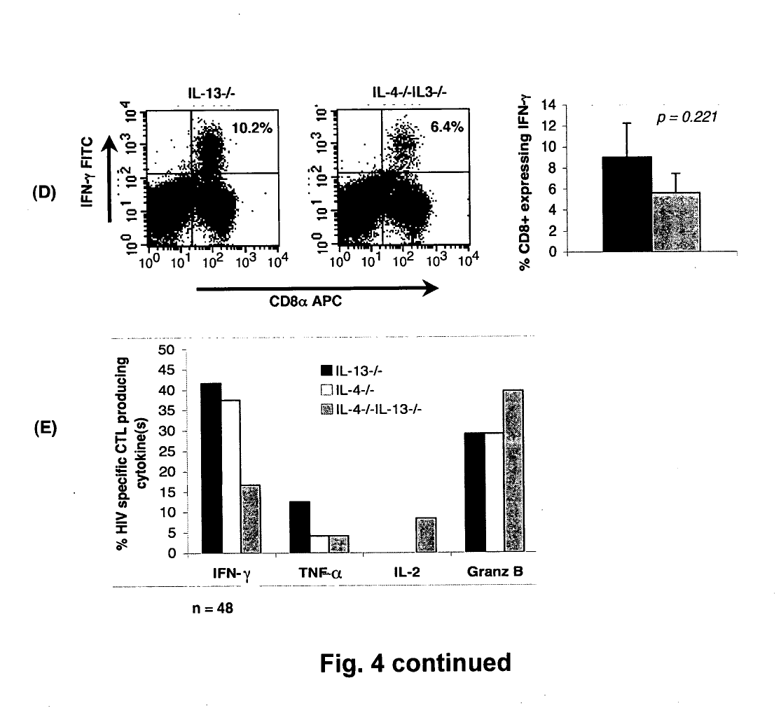

FIG. 4 shows the results of studies examining effector CTL responses in Th2 cytokine and STAT6 KO mice compared with responses in wild type BALB/c. Fourteen days post i.n./i.m. poxvirus prime boost immunisation splenocytes from IL-4^{-/-} (white), IL-13^{-/-} (striped), STAT6^{-/-} (grey) and wild type BALB/c (black) (n=4-6 per group) were harvested and K^{d}Gag₁₉₇₋₂₀₅-specific T-cell responses were measured by tetramer staining (results shown in FIG. 4A) and following AMQMLKETI gag peptide stimulation by IFN-γ ELISpot (results shown in FIG. 4B), the methods as described in the Materials and methods section. For ELISpot, the un-stimulated cells from each sample were used as the background control and this value was subtracted from each sample before plotting the data. *p=0.0024, **p=0.0174 and ***p=0.0200 as determined using the Student's t-test. The data represent mean+SD. The data are representative of at least three experiments.

FIG. 4C shows the results of K^{d}Gag₁₉₇₋₂₀₅-specific effector CTL avidity in Th2 cytokine and STAT6 KO mice. Fourteen days following i.n./i.m. poxvirus prime boost immunisation, the percentage of K^{d}Gag₁₉₇₋₂₀₅ CD8⁺ splenocyte loss (dissociation) was measured as described in the Materials and methods section. The data represent mean±SD obtained using four mice per group and p values are calculated at the 60 minutes end time point using two-tailed, two-sample equal variance Student's t-test. Experiments were repeated at least three times.

In FIGS. 4D and 4E, the results of analysis of cytokine expression in IL-13^{-/-}, IL-14^{-/-} and IL-4^{-/-}IL-13^{-/-} K^{d}G₁₉₆₋₂₀₅-specific effector CTL are shown. Mice (n=3-4 per group) were immunised i.n./i.m. with AE FPV/AE VV and at 14 days prime boost. In FIG. 4D, IFN-γ protein expression in CD8⁺ T cells was measured by ICS and in FIG. 4E, IFN-γ, TNF-α, IL-2 and granzyme B mRNA expression in K^{d}Gag⁺₁₉₇₋₂₀₅ single splenocytes (n=48) was assessed by single-cell multiplex nested PCR. In FIG. 4D, the representative flow cytometry plots are from individual mice per group, with the percentage IFN-γ CD8⁺ T cells given in the upper right quadrant, and the graph presents the mean percentage IFN-γ⁺ CD8⁺+SD obtained using 3-4 mice per group. Black bars represent IL-13^{-/-} mice and grey bars represent IL-4^{-/-}IL-13^{-/-} mice. In FIG. 4E, single-cell data are representative of two experiments.

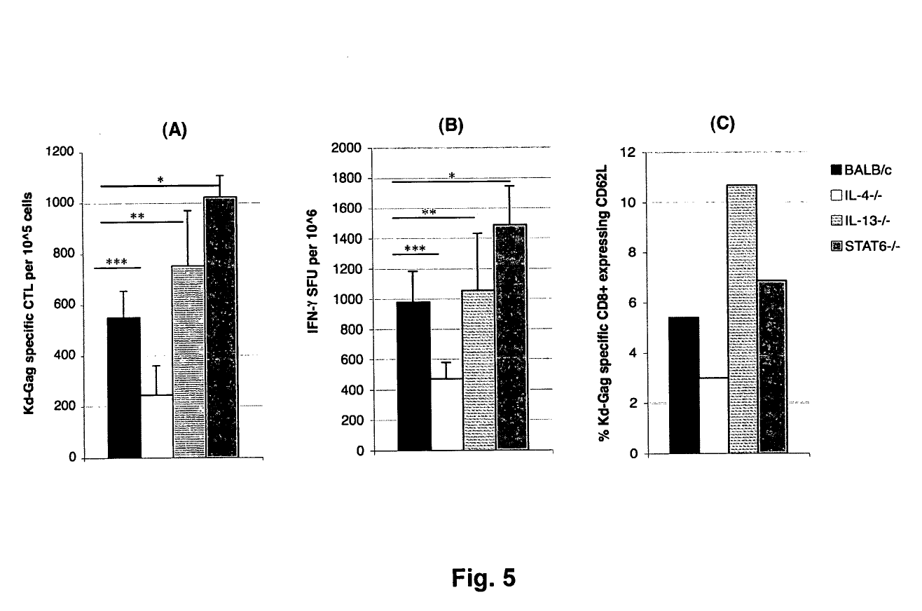

FIG. 5 shows memory CTL responses in Th2 cytokine and STAT6 KO mice compared with responses in wild type BALB/c mice. Eight weeks following i.m./i.m. poxvirus prime boost immunisation, memory was recalled i.r. with AE VV. At 7 days post recall splenocytes from IL-4^{-/-} (white), IL-13^{-/-} (striped), STAT6^{-/-} (grey) and wild type BALB/c (black) were harvested and K^{d}Gag₁₉₇₋₂₀₅-specific memory T-cell responses were measured by tetramer staining (*p=0.0012, **p=0.23, ***p=0.008) (as shown in FIG. 5A), IFN-γ ELISpot (*p=0.0206, **p=0.6954 and ***p=0.0047) (as shown in FIG. 5B) and CD8α, CD62L staining and flow cytometry (as shown in FIG. 5C), the method as described in the Materials and methods section. In FIGS. 5A and 5B, the data represent mean+SD of 4 mice per group and p values were determined using two-tailed, two-sample equal variance Student's t-test. In FIG. 5C, K^{d}Gag₁₉₇₋₂₀₅ CD8⁺ CD62L⁺ data are from pooled splenocytes. When plotting ELISpot and flow cytometry data, un-stimulated cells from each sample were used as the background control and this value was subtracted from each sample.

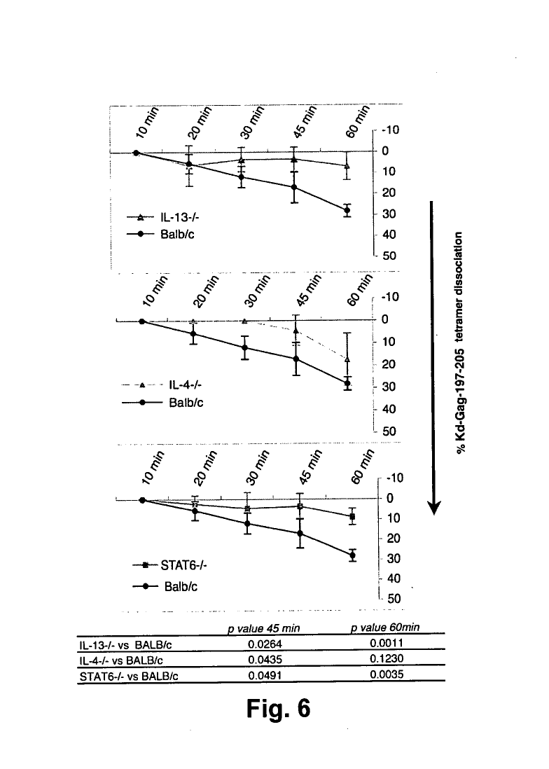

FIG. 6 shows the results of studies examining K^{d}Gag₁₉₇₋₂₀₅-specific memory CTL avidity in Th2 cytokine and STAT6 KO mice. Eight weeks following i.m./i.m. poxvirus prime boost immunisation, memory was recalled i.r. using AE VV. At 7 days, percentage of K^{d}Gag⁺₁₉₇₋₂₀₅ CD8⁺ splenocyte loss (dissociation) was measured as described in the Materials and methods section. The data represent mean±SD obtained with 3-4 mice per group and tetramer loss p values are calculated at 45 minutes and the 60 minutes end time point using two-tailed, two-sample equal or unequal variance Student's t-test and shown in the bottom panel. The data are representative of at least three experiments.

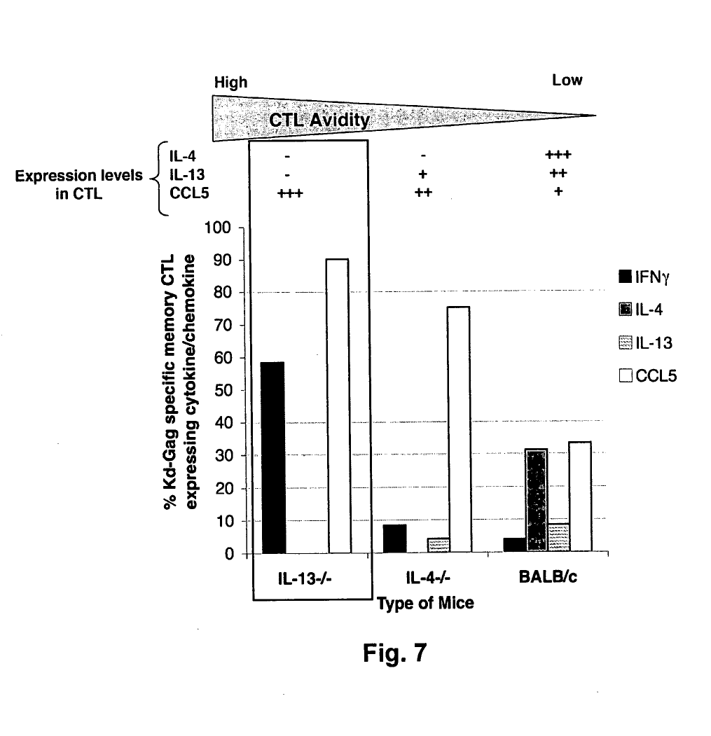

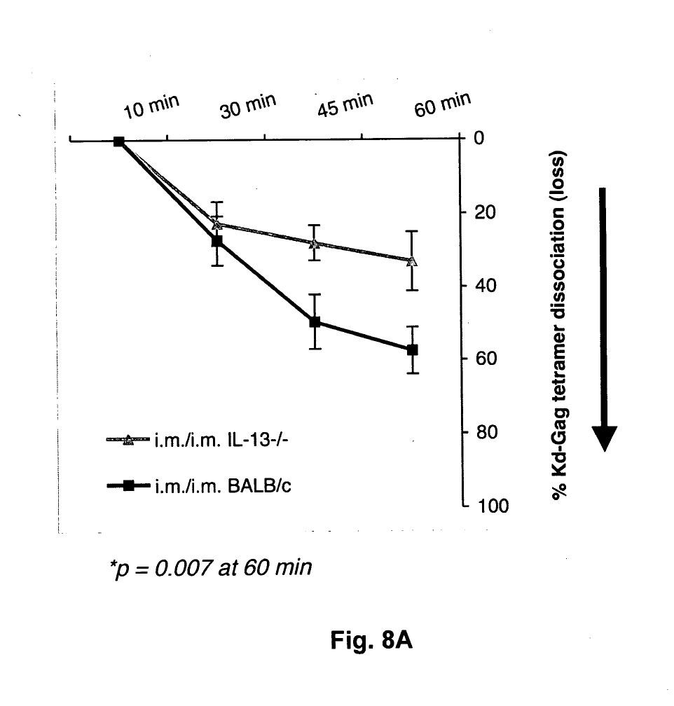

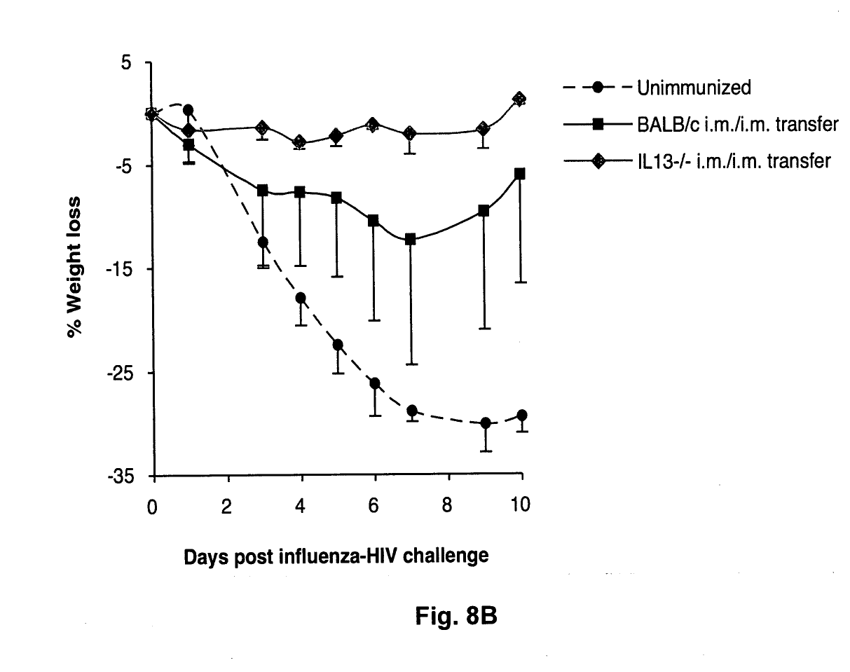

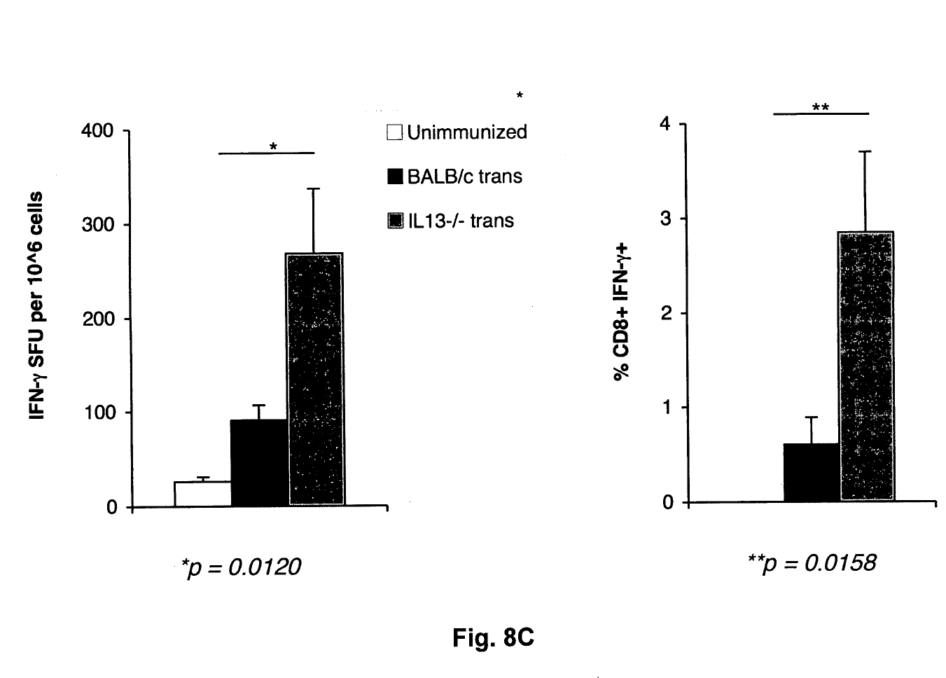

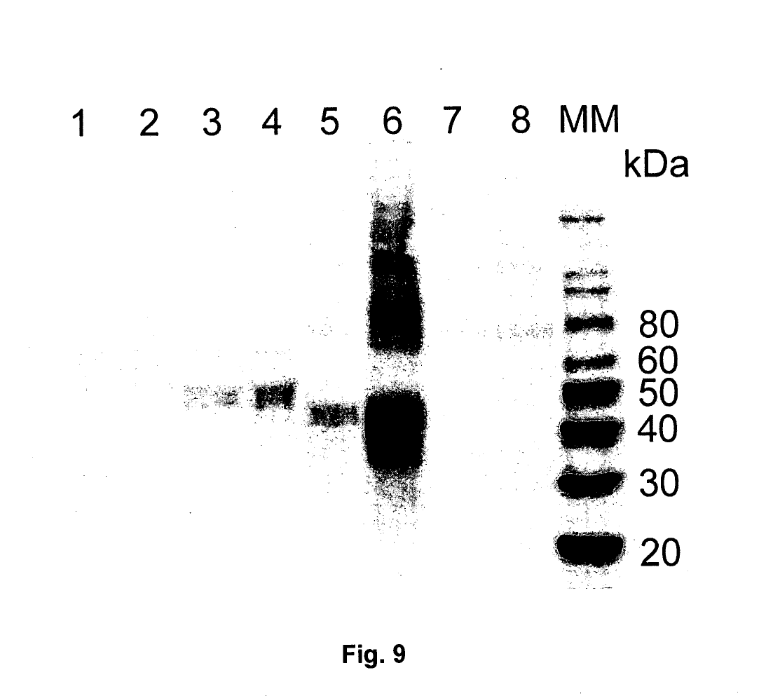

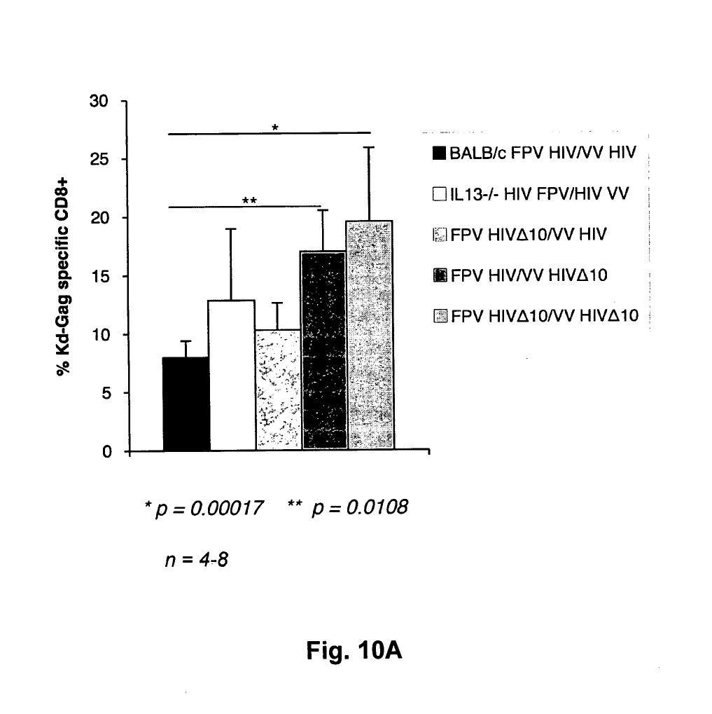

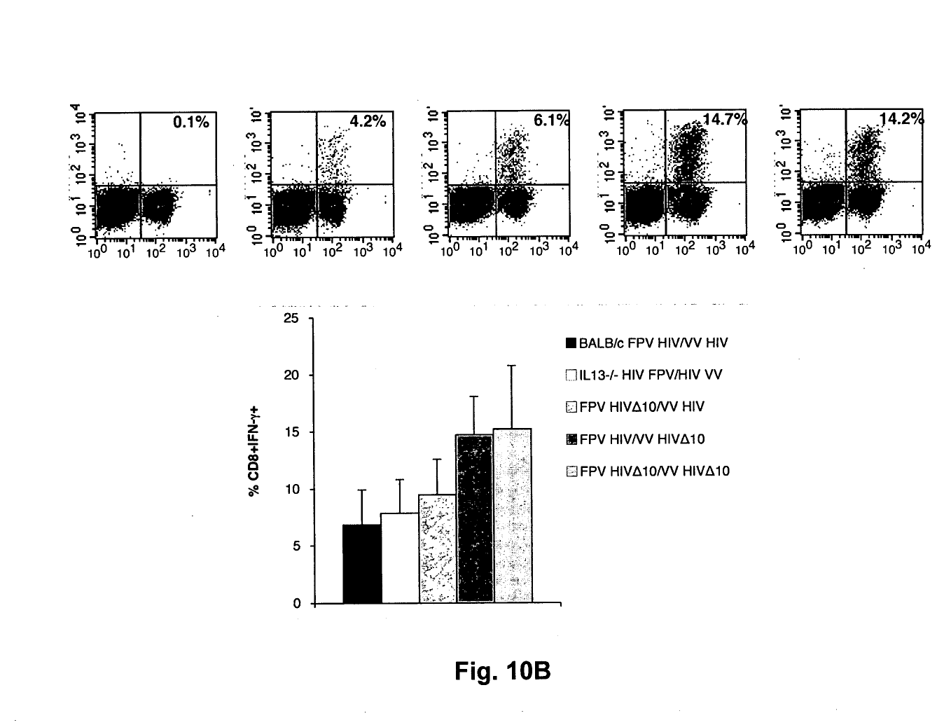

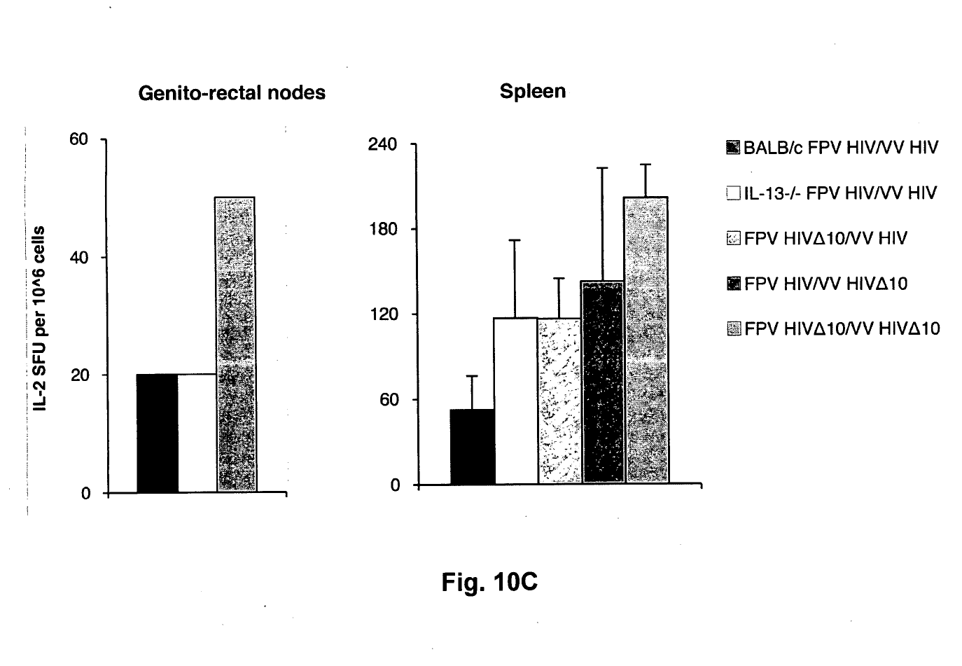

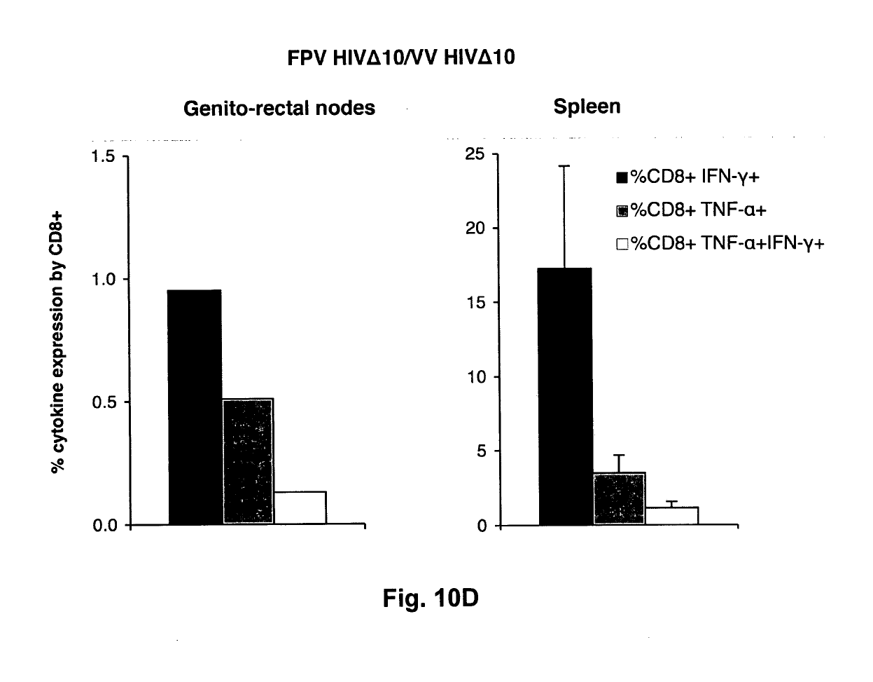

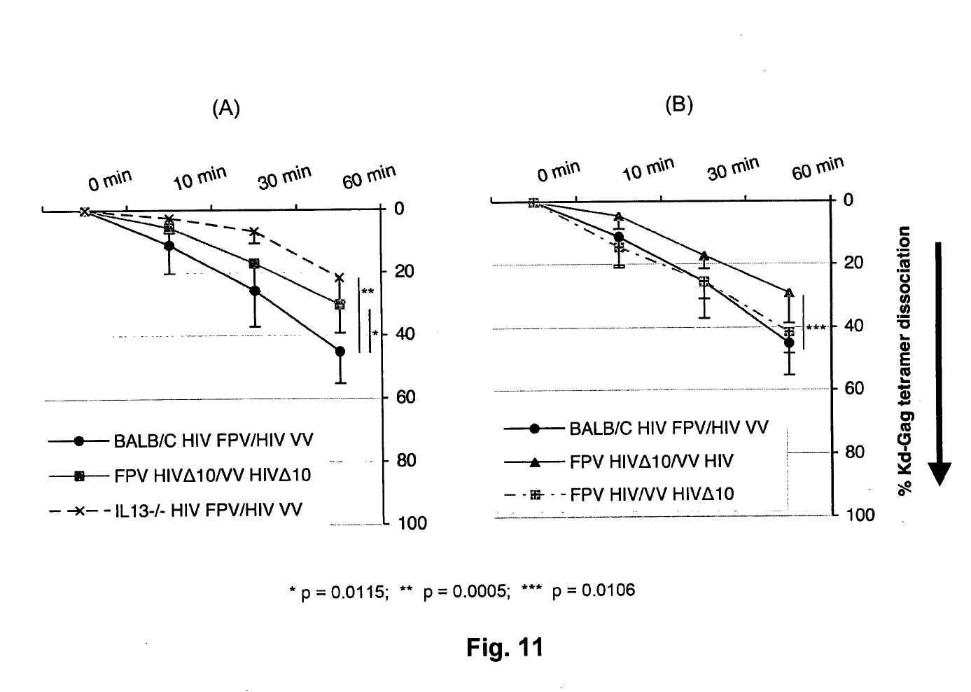

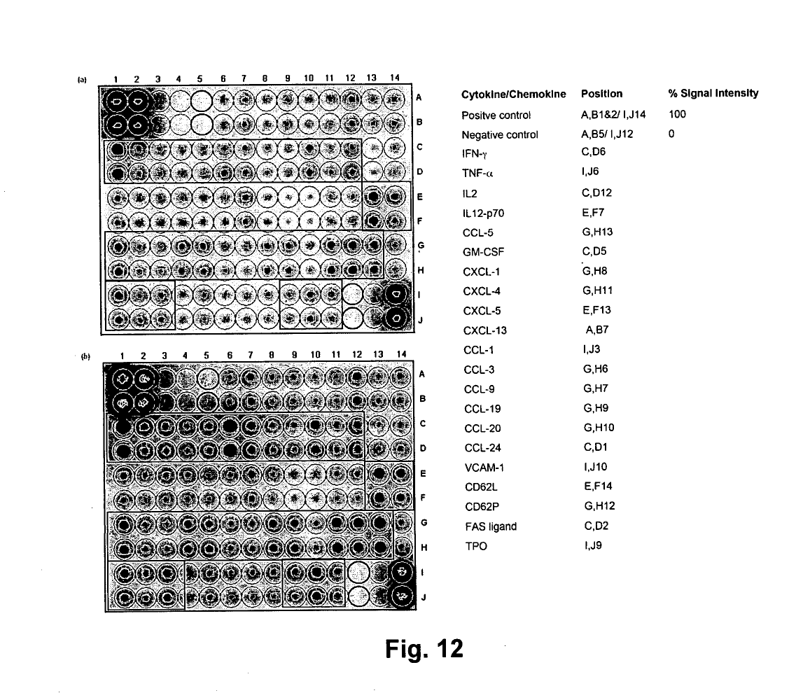

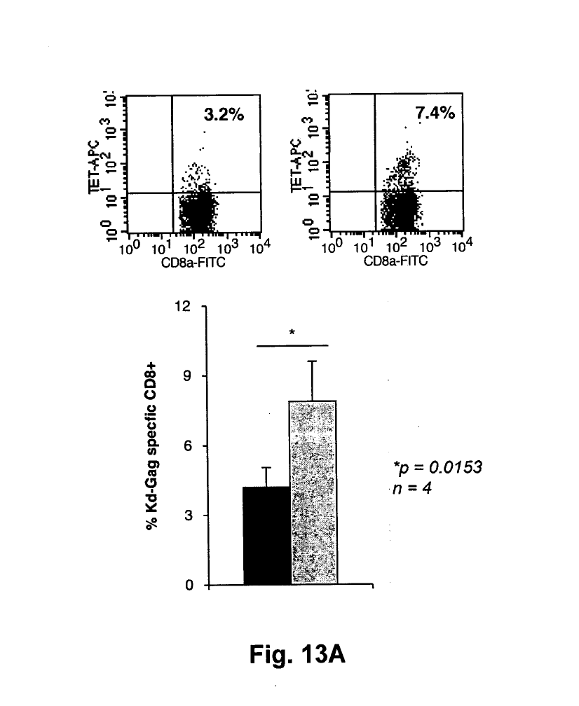

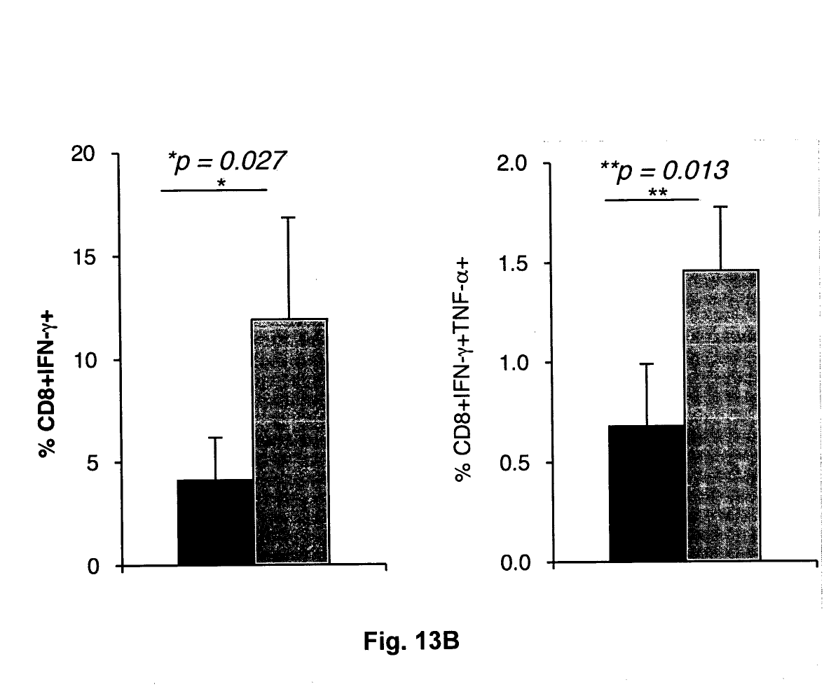

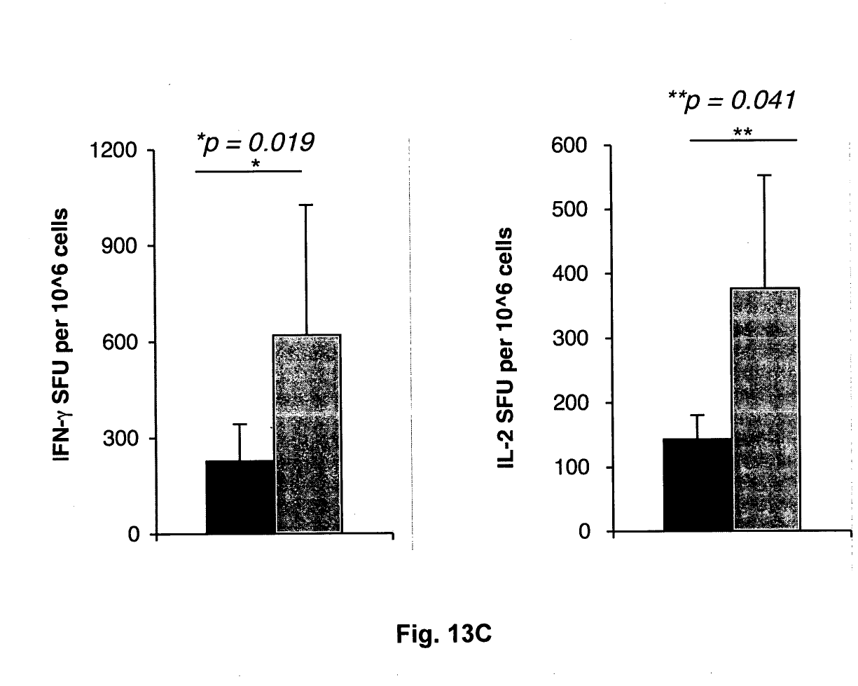

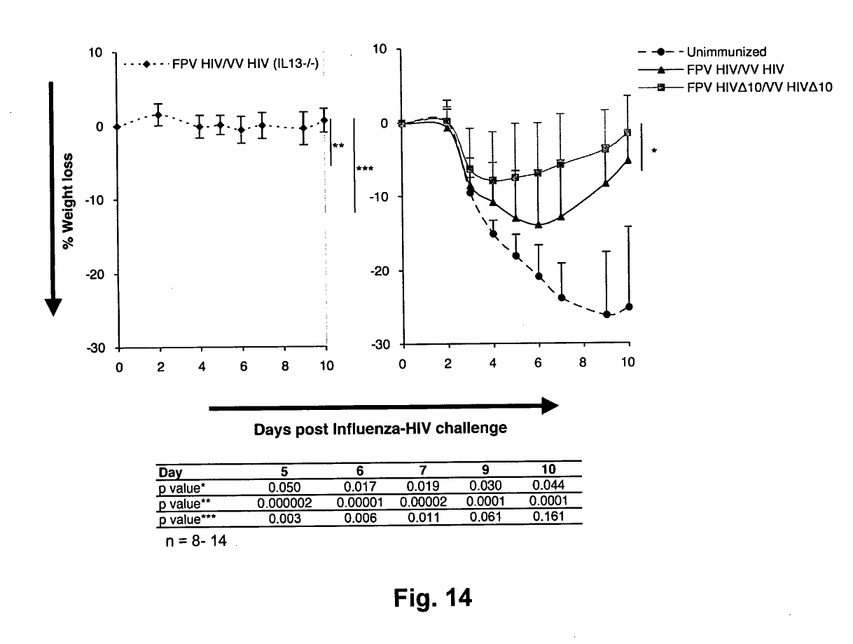

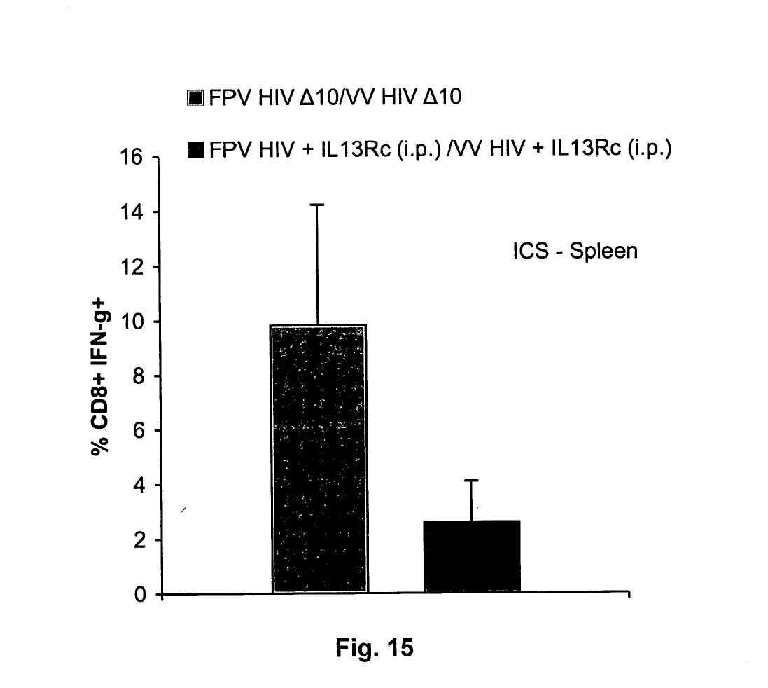

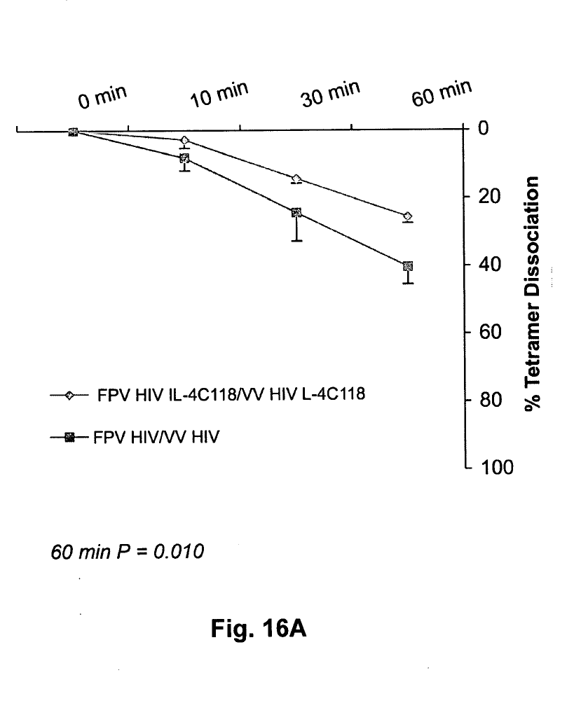

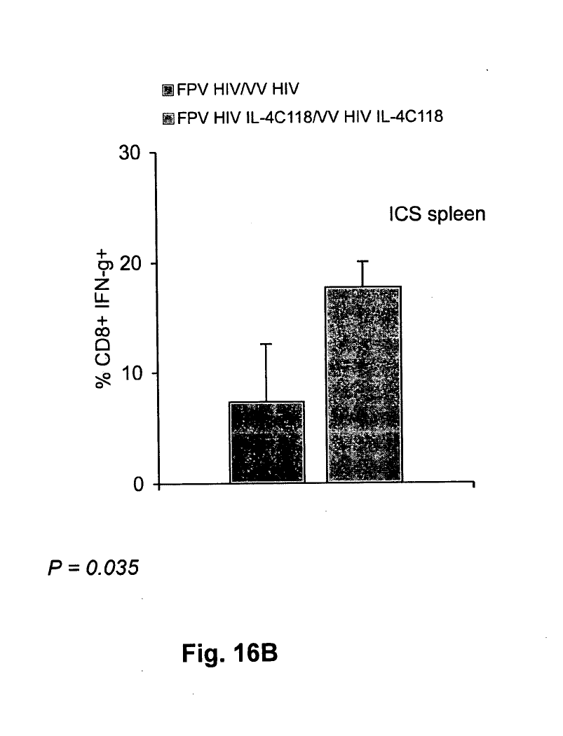

FIG. 7 shows a schematic diagram of cytokine/chemokine expression and memory CTL avidity. Top of the diagram indicates CTL avidity and its correlation with IL-3, IL-4 and CCL5 expression by K^{d}Gag₁₉₇₋₂₀₅-specific CTL. Symbols indicate (+++) high, (++) medium, (+) low or (−) no expression of the indicated cytokine or chemokine. The graph details percentage of IL-13^{-/-}, IL-4^{-/-} and wild type BALB/c K^{d}Gag₁₉₇₋₂₉₅-specific single splenocytes (n=48), expressing IFN-γ IL-4, IL-13 or CCL5, 8 wk following i.m./i.m. poxvirus prime boost immunisation, and i.r. memory recall as described in the Materials and methods section. The box superimposed on the graph highlights the cytokine and CCL5 expression profile of high-avidity CTL.

FIGS. 8-16 show the results of studies described in Example 2.

FIG. 8A shows the tetramer dissociation (avidity) of the transferred control and IL-13^{-/-} GKO spleen cells from prime boost immunised (i.m./i.m.) mice.

FIG. 8B shows the capacity of transferred CD8⁺ T cells from prime-boost immunised normal control mice or IL-13^{-/-} GKO mice to protect against a challenge with influenza virus encoding K^{d}Gag₁₉₇₋₂₀₅ specific HIV-1 epitope. Balb/c control mice or IL-13^{-/-} GKO mice were immunised (i.m./i.m.) with recombinant FPV and VV vectors expressing HIV-1 antigens. Fourteen days after the boost immunisation 1×10⁷ spleen cells were transferred to naïe Balb/c mice which were subsequently challenged i.n. with a recombinant influenza virus encoding K^{d}Gag₁₉₇₋₂₀₅ epitope and the weight loss through influenza infection recorded daily to monitor for protection.

FIG. 8C shows the results of the transfer study described in Example 2. Ten days after influenza K^{d}Gag₁₉₇₋₂₀₅ challenge the spleens from mice receiving CD8⁺ T cells from normal or IL-13^{-/-} GKO mice were stimulated with 9-mer gag peptide and the T cell responses measured by IFN-γ ELISpot and IFN-γ intracellular staining.

FIG. 9 shows an immuno-blot of recombinant poxviruses expressing mouse sIL-13Rα2. Media recovered from infected cells was in lanes 1 to 4. Infected cell lysates are in lanes 5 to 8. FPV-086 was in lanes 1 and 8. VV-336 in lanes 2 and 7. VV-IL-13Rα2Δ10 was in lanes 3 and 6. FPV-IL-13Rα2Δ10 was in lanes 4 and 5. The standards were (MM) MagicMark XP Western Protein Standard (Invitrogen, LC5603). Primary antibody was goat anti-mouse IL-13Rα2 (R&D Systems, AF539).

FIG. 10A shows the number of antigen-specific (tetramer-positive) CD8⁺ T cells in spleen induced by FPV and VV vectors co-expressing IL-13R decoy receptor either in the priming or boosting vector. Results are 14 days after i.n./i.m. prime boost immunisation (control versus IL-13R vaccination).

FIG. 10B shows the number of IFN-γ intracellular staining CD8⁺ T cells in spleen induced by FPV and VV vectors co-expressing IL-13R decoy receptor either in the priming or boosting vector. Results are 14 days after i.n./i.m. prime boost immunisation (control versus IL-13R vaccination).

FIG. 10C shows the effect of co-expression of IL-13R decoy receptor on the induction of CD8⁺ T cells expressing IL-2 from spleen and genito-rectal nodes, indicating the induction of polyfunctional T cells. Results are from ELIspot 14 days after i.n./i.m. prime boost immunisation (control versus IL-13R vaccination).

FIG. 10D shows the expression of multiple cytokines (TNF and IFN-γ) in CM⁺ T cells from control immunised mice and mice immunised with IL-13R decoy receptor recombinant vectors (measured 14 days following i.n./i.m. prime-boost immunisation).

FIG. 11 shows measurement of CD8⁺ T cell avidity of splenocytes from mice immunised with recombinant FPV and VV vectors co-expressing IL-13R decoy receptor and HIV-1 antigens. The figure compares the IL-13R delivered in the priming or boosting vector with IL-13^{-/-} GKO mice. Results are 14 days after i.n./i.m. prime boost immunisation (control versus IL-13R vaccination).

FIG. 12 shows an antibody array of multiple cytokines and chemokines of CD8⁺ T cells from splenocytes from mice immunised with vaccine vectors co-expressing HIV-1 genes and IL-13R decoy receptor, indicating greatly increased polyfunctional activity from those given the IL-13R decoy encoding vectors. Figures shows control (top—(a)) and IL-13R (bottom—(b)) vaccination measured 14 days following i.n./i.m. prime boost immunisation (using Ray Bio Mouse 64 cytokine antibody array).

FIG. 13A shows antigen-specific (tetramer-positive) memory CD8⁺ T cells (8 weeks following i.n./i.m. prime boost immunisation) from mice immunised with control vaccine vectors or vectors co-expressing IL-13R decoy receptors.

FIG. 13B shows IFN-γ/TNF-α intracellular staining of CD8⁺ memory T cells 8 weeks following i.n./i.m. prime boost immunisation with either control vaccine or vectors co-expressing IL-13R decoy receptor (ELIspot).

FIG. 13C shows IFN-γ/IL-2 intracellular staining of CD8⁺ memory T cells 8 weeks following i.n./i.m. prime boost immunisation with either control vaccine or vectors co-expressing IL-13R decoy receptor (ELIspot).

FIG. 14 shows the ability of FPV and VV vectors co-expressing IL-13R decoy receptors to elicit superior protective immunity compared to control vectors. Mice were immunised (i.n./i.m. prime boost immunisation) with control vectors or vectors co-expressing HIV-1 antigens and IL-13R decoy receptor and challenged three weeks later with an influenza virus encoding a dominant HIV-1K^{d}Gag₁₉₇₋₂₀₅ T cell epitope. The weights were monitored for ten days.

FIG. 15 shows IFN-γ intracellular cytokine staining comparing co-expression of IL-13 soluble receptor compared to IL-13 receptor delivered i.p. at the time of immunisation (mice were immunised i.n./i.m. two weeks apart, and responses were evaluated fourteen days post booster immunisation). The results in this figure demonstrate the lack of ability of IL-13Rα2 recombinant soluble protein given 10 μg/mouse (RND Systems) to influence CD8⁺ T cell responses.

FIG. 16A shows the effect of IL-4 antagonist IL-4C118 expressed by recombinant vectors to influence the avidity of CD8⁺ T cells (tetramer dissociation). Mice were immunised i.n./i.m. two weeks apart, and responses were evaluated fourteen days post booster immunisation. Enhanced avidity is seen in mice receiving IL-C118 expressing vectors.

FIG. 16B shows the effect of effect of IL-4 antagonist IL-4C 118 expressed by recombinant vectors to influence the number of CD8⁺ T cells (IFN-γ intracellular cytokine staining or ICS) elicited by prime boost vaccination. Mice were immunised i.n./i.m. two weeks apart, and responses were evaluated fourteen days post booster immunisation.

### DETAILED DESCRIPTION OF THE INVENTION

### I. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” is used herein to refer to conditions (e.g., amounts, concentrations, time etc) that vary by as much as 15%, 14%, 13%, 12%, 11%, 10%, 9%, 8%, 7%, 6%, 5%, 4%, 3%, 2%, or 1% to a specified condition.

By “antigen” is meant all, or part of, a protein, peptide, or other molecule or macromolecule capable of eliciting an immune response in a vertebrate animal, especially a mammal. Such antigens are also reactive with antibodies from animals immunised with that protein, peptide, or other molecule or macromolecule.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

By “autologous” is meant something (e.g., cells, tissues etc) derived from the same organism.

The term “allogeneic” as used herein refers to cells, tissues, organisms etc that are of different genetic constitution.

By “biologically active fragment” is meant a fragment of a full-length parent polypeptide which fragment retains an activity of the parent polypeptide. As used herein, the term “biologically active fragment” includes deletion mutants and small peptides, for example of at least 6, 7, 8, 9, 10, 11, 12, 13, 14, 15, 16, 17, 18, 19, 20, 21, 22, 23, 24, 25, 26, 27, 28, 29 or 30 contiguous amino acids, which comprise an activity of the parent polypeptide. Peptides of this type may be obtained through the application of standard recombinant nucleic acid techniques or synthesised using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

As used herein, a “cellular composition,” “cellular vaccine” or “cellular immunogen” refers to a composition comprising at least one cell population as an active ingredient.

As used herein, the term “cis-acting sequence” or “cis-regulatory region” or similar term shall be taken to mean any sequence of nucleotides which is derived from an expressible genetic sequence wherein the expression of the genetic sequence is regulated, at least in part, by the sequence of nucleotides. Those skilled in the art will be aware that a cis-regulatory region may be capable of activating, silencing, enhancing, repressing or otherwise altering the level of expression and/or cell-type-specificity and/or developmental specificity of any structural gene sequence.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

The terms “construct” and “synthetic construct” are used interchangeably herein to refer to heterologous nucleic acid sequences that are operably linked to each other and may include sequences providing the expression of a polynucleotide in a host cell and optionally sequences that provide for the maintenance of the construct.

By “corresponds to” or “corresponding to” is meant an antigen which encodes an amino acid sequence that displays substantial similarity to an amino acid sequence in a target antigen. In general the antigen will display at least about 30, 40, 50, 55, 60, 65, 70, 75, 80, 85, 90, 91, 92, 93, 94, 95, 96, 97, 98, 99% similarity to at least a portion of the target antigen.

As used herein, “culturing,” “culture” and the like refer to the set of procedures used in vitro where a population of cells (or a single cell) is incubated under conditions which have been shown to support the growth or maintenance of the cells in vitro. The art recognises a wide number of formats, media, temperature ranges, gas concentrations etc. which need to be defined in a culture system. The parameters will vary based on the format selected and the specific needs of the individual who practices the methods herein disclosed. However, it is recognised that the determination of culture parameters is routine in nature.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules.

By “effective amount”, in the context of modulating an immune response or treating or preventing a disease or condition, is meant the administration of that amount of composition to an individual in need thereof, either in a single dose or as part of a series, that is effective for that modulation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “expression vector” is meant any autonomous genetic element capable of directing the synthesis of a protein encoded by the vector. Such expression vectors are known by practitioners in the art.

The term “gene” is used in its broadest context to include both a genomic DNA region corresponding to the gene as well as a cDNA sequence corresponding to exons or a recombinant molecule engineered to encode a functional form of a product.

To enhance immune response (“immunoenhancement”), as is well-known in the art, means to increase the animal's capacity to respond to foreign or disease-specific antigens (e.g., cancer antigens) i.e., those cells primed to attack such antigens are increased in number, activity, and ability to detect and destroy the those antigens. Strength of immune response is measured by standard tests including: direct measurement of peripheral blood lymphocytes by means known to the art; natural killer cell cytotoxicity assays (see, e.g., Provinciali M. et al. (1992, J. Immunol. Meth. 155: 19-24), cell proliferation assays (see, e.g., Vollenweider, I. And Groseurth, P. J. (1992, J. Immunol. Meth. 149: 133-135), immunoassays of immune cells and subsets (see, e.g., Loeffler, D. A., et al. (1992, Cytom. 13: 169-174); Rivoltini, L., et al. (1992, Can. Immunol. Immunother. 34: 241-251); or skin tests for cell-mediated immunity (see, e.g., Chang, A. E. et al. (1993, Cancer Res. 53: 1043-1050). Any statistically significant increase in strength of immune response as measured by the foregoing tests is considered “enhanced immune response”, “immunoenhancement” or “immunopotentiation” as used herein. Enhanced immune response is also indicated by physical manifestations such as fever and inflammation, as well as healing of systemic and local infections, and reduction of symptoms in disease, i.e., decrease in tumor size, alleviation of symptoms of a disease or condition including, but not restricted to, leprosy, tuberculosis, malaria, naphthous ulcers, herpetic and papillomatous warts, gingivitis, arthrosclerosis, the concomitants of AIDS such as Kaposi's sarcoma, bronchial infections, and the like. Such physical manifestations also define “enhanced immune response” “immunoenhancement” or “immunopotentiation” as used herein.

Reference herein to “immunodeficient” includes reference to any condition in which there is a deficiency in the production of humoral and/or cell-mediated immunity.

Reference herein to “immuno-interactive” includes reference to any interaction, reaction, or other form of association between molecules and in particular where one of the molecules is, or mimics, a component of the immune system.

“Inactivation” of a cell is used herein to indicate that the cell has been rendered incapable of cell division to form progeny. The cell may nonetheless be capable of response to stimulus, or biosynthesis and/or secretion of cell products such as cytokines. Methods of inactivation are known in the art. Preferred methods of inactivation are treatment with toxins such as mitomycin C, or irradiation. Cells that have been fixed or permeabilised and are incapable of division are also examples of inactivated cells.

By “isolated” is meant material that is substantially or essentially free from components that normally accompany it in its native state.

A composition is “immunogenic” if it is capable of either: a) generating an immune response against an antigen (e.g., a tumor antigen) in a naïve individual; or b) reconstituting, boosting, or maintaining an immune response in an individual beyond what would occur if the compound or composition was not administered. A composition is immunogenic if it is capable of attaining either of these criteria when administered in single or multiple doses.

By “modulating” is meant increasing or decreasing, either directly or indirectly, the level and/or functional activity of a target molecule. For example, an agent may indirectly modulate the said level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide. In certain embodiments, “modulation” or “modulating” means that a desired/selected response is more efficient (e.g., at least 10%, 20%, 30%, 40%, 50%, 60% or more), more rapid (e.g., at least 10%, 20%, 30%, 40%, 50%, 60% or more), greater in magnitude (e.g., at least 10%, 20%, 30%, 40%, 50%, 60% or more), and/or more easily induced (e.g., at least 10%, 20%, 30%, 40%, 50%, 60% or more) than if the antigen had been used alone.

The term “5′ non-coding region” is used herein in its broadest context to include all nucleotide sequences which are derived from the upstream region of an expressible gene, other than those sequences which encode amino acid residues which comprise the polypeptide product of said gene, wherein 5′ non-coding region confers or activates or otherwise facilitates, at least in part, expression of the gene.

By “obtained from” is meant that a sample such as, for example, a nucleic acid extract or polypeptide extract is isolated from, or derived from, a particular source of the host. For example, the extract may be obtained from a tissue or a biological fluid isolated directly from the host.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

The term “operably connected” or “operably linked” as used herein means placing a structural gene under the regulatory control of a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e., the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the genes from which it is derived.

The terms “patient,” “subject,” “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc), marine mammals (e.g., dolphins, whales), reptiles (e.g., snakes, frogs, lizards etc), and fish. A preferred subject is a human in need of treatment or prophylaxis for a condition or disease, which is associated with the presence or aberrant expression of an antigen of interest. However, it will be understood that the aforementioned terms do not imply that symptoms are present.

By “pharmaceutically-acceptable carrier” is meant a solid or liquid filler, diluent or encapsulating substance that may be safely used in topical or systemic administration.

The term “pharmaceutically compatible salt” as used herein refers to a salt which is toxicologically safe for human and animal administration. This salt may be selected from a group including hydrochlorides, hydrobromides, hydroiodides, sulphates, bisulphates, nitrates, citrates, tartrates, bitartrates, phosphates, malates, maleates, napsylates, fumarates, succinates, acetates, terephthalates, pamoates and pectinates.

The term “polynucleotide” or “nucleic acid” as used herein designates mRNA, RNA, cRNA, cDNA or DNA. The term typically refers to oligonucleotides greater than 30 nucleotides in length.

The terms “polynucleotide variant” and “variant” refer to polynucleotides displaying substantial sequence identity with a reference polynucleotide sequence or polynucleotides that hybridise with a reference sequence under stringent conditions that are defined hereinafter. These terms also encompasses polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide,” “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides which vary from a reference polypeptide by the addition, deletion or substitution of at least one amino acid. It is well understood in the art that some amino acids may be changed to others with broadly similar properties without changing the nature of the activity of the polypeptide. Preferred variant polypeptides comprise conservative amino acid substitutions. Exemplary conservative substitutions in a polypeptide may be made according to the following table:

TABLE A

| ORIGINAL RESIDUE | EXEMPLARY SUBSTITUTIONS |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in TABLE A. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Asn) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a smaller side chain (e.g., Ala, Ser) or no side chain (e.g., Gly) is substituted for, or by, one having a bulky side chain (e.g., Phe or Trp).

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene. Preferred promoters according to the invention may contain additional copies of one or more specific regulatory elements to further enhance expression in a cell, and/or to alter the timing of expression of a structural gene to which it is operably connected.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

As used herein “stimulating” an immune or immunological response refers to administration of a composition that initiates, boosts, or maintains the capacity for the host's immune system to react to a target substance or antigen, such as a foreign molecule, an allogeneic cell, or a tumour cell, at a level higher than would otherwise occur. Stimulating a “primary” immune response refers herein to eliciting specific immune reactivity in a subject in which previous reactivity was not detected; for example, due to lack of exposure to the target antigen, refractoriness to the target, or immune suppression. Stimulating a “secondary” response refers to the reinitiation, boosting, or maintenance of reactivity in a subject in which previous reactivity was detected; for example, due to natural immunity, spontaneous immunisation, or treatment using one or several compositions or procedures.

By “treatment,” “treat,” “treated” and the like is meant to include both prophylactic and therapeutic treatment, including but not limited to preventing, relieving, altering, reversing, affecting, inhibiting the development or progression of, ameliorating, or curing (1) a disease or condition associated with the presence or aberrant expression of a target antigen, or (2) a symptom of the disease or condition, or (3) a predisposition toward the disease or condition, including conferring protective immunity to a subject.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

### 2. Compositions

The present invention stems at least in part from the determination that expression of the cytokine IL-13 plays an important role in down-regulating the functional avidity of cytotoxic T-cells, and that T-cell avidity is improved by inhibition of IL-13 function in the local milieu of the immune response, leading the inventors to discover that a subject's T-cell mediated immune response may be enhanced by removal, inhibition or neutralisation of IL-13 production or function in the local milieu of the immune response.

Based on these observations, the present inventors propose that more efficacious prophylactic or therapeutic immune responses against a target antigen can be achieved using compositions that comprise a first agent comprising an immune stimulator that stimulates or otherwise enhances an immune response to the target antigen or a polynucleotide sequence encoding an immune stimulator that stimulates or otherwise enhances an immune response to the target antigen together with a second agent comprising an inhibitor of IL-13 function or a polynucleotide sequence encoding an inhibitor of IL-13 function. In specific embodiments, the compositions are introduced into antigen-presenting cells such that the first and second agents are co-located in or on or co-presented by the antigen-presenting cells.

The present inventors have also observed that inhibitors of IL-4 function may achieve a more efficacious prophylactic or therapeutic immune responses against a target antigen. Thus in some embodiments, the present invention contemplates an inhibitor of IL-4 function or a polynucleotide sequence encoding an inhibitor of IL-4 function in substitution of an inhibitor of IL-13 function or a polynucleotide sequence encoding an inhibitor of IL-13 function.

2.1 Inhibitors of IL-13 Function

The inhibitor of IL-13 function includes any molecule or compound that directly or indirectly binds or physically associates with IL-13 or its receptor(s) and that suitably blocks, inhibits or otherwise antagonises at least one of its functions or activities (e.g., binding to or interaction with one or more surface molecules (e.g., receptors) present on white blood cells, especially lymphocytes and more especially T lymphocytes). The binding or association may involve the formation of an induced magnetic field or paramagnetic field, covalent bond formation, an ionic interaction such as, for example, occur in an ionic lattice, a hydrogen bond or alternatively, a van der Waals interaction such as, for example, a dipole-dipole interaction, dipole-induced-dipole interaction, induced-dipole-induced-dipole interaction or a repulsive interaction or any combination of the above forces of attraction.

In certain embodiments, the inhibitor of IL-13 function is any molecule capable of specifically preventing activation of cellular receptors for IL-13. For example, inhibitors of this type can be selected from soluble, membrane-bound or defective IL-13 receptors or soluble IL-13 receptor subunits, including but not limited to IL-13Rα2 and IL-13Rα2Δ10.

In certain embodiments, the inhibitor of IL-13 function is a modified, mutated or defective form of IL-13 or IL-4, including but not limited to IL-4C118 or AEROVANT™ (AER 001, pitrakinra produced by Aerovance) which is a 15 kDa recombinant human IL-4 mutein (see The Lancet (2007) 370: 1422-1431).

Alternatively, such an inhibitor can be an antigen-binding molecule that is immuno-interactive with an IL-13 receptor. In these embodiments, the antigen-binding molecule may bind to the IL-13 receptor but will not signal via the receptor, thus blocking any host IL-13 signalling. In other embodiments, the inhibitor of IL-13 function is an antigen-binding molecule that is immuno-interactive with at least a portion of IL-13. In these embodiments, the antigen-binding molecules can be immuno-interactive with an active or an inactive form of IL-13, the difference being that antigen-binding molecules to the active cytokine are more likely to recognise epitopes that are only present in the active conformation. Representative examples of such inhibitors include ligands or single-chain antibodies including those disclosed in US published patent application no. 2009-0060916 A1, and the antibodies disclosed in US published patent application no. 2005-0186146 A1.

In some embodiments, the inhibitor of IL-13 function is an IL-13 trap, including but not limited to those disclosed in US published patent application no. 2003-0211104 A1.

2.2 Inhibitors of IL-4 Function

The present inventors have also observed that inhibitors of IL-4 function may achieve a more efficacious prophylactic or therapeutic immune responses against a target antigen. Thus in some embodiments, the present invention contemplates the inclusion of a third agent comprising an inhibitor of IL-4 function or a polynucleotide sequence encoding an inhibitor of IL-4 function.

The inhibitor of IL-4 function includes any molecule or compound that directly or indirectly binds or physically associates with IL-4 or its receptor(s) and that suitably blocks, inhibits or otherwise antagonises at least one of its functions or activities (e.g., binding to or interaction with one or more surface molecules (e.g., receptors) present on white blood cells, especially lymphocytes and more especially T lymphocytes). The binding or association may involve the formation of an induced magnetic field or paramagnetic field, covalent bond formation, an ionic interaction such as, for example, occur in an ionic lattice, a hydrogen bond or alternatively, a van der Waals interactions such as, for example, a dipole-dipole interaction, dipole-induced-dipole interaction, induced-dipole-induced-dipole interaction or a repulsive interaction or any combination of the above forces of attraction.

In certain embodiments, the inhibitor of IL-4 function is any molecule capable of specifically preventing activation of cellular receptors for IL-4. For example, inhibitors of this type can be selected from soluble or defective IL-4 receptors or soluble IL-4 receptor subunits.

In certain embodiments, the inhibitor of IL-13 function is a modified, mutated or defective form of IL-4, including but not limited to IL-4C118 or AEROVANT™ (AER 001, pitrakinra produced by Aerovance) which is a 15 kDa recombinant human IL-4 mutein (see The Lancet (2007) 370: 1422-1431).

Alternatively, such an inhibitor can be an antigen-binding molecule that is immuno-interactive with an IL-4 receptor. In these embodiments, the antigen-binding molecule may bind to the IL-13 receptor but will not signal via the receptor, thus blocking any host IL-13 signalling. In other embodiments, the inhibitor of IL-4 function is an antigen-binding molecule that is immuno-interactive with at least a portion of IL-4. In these embodiments, the antigen-binding molecules can be immuno-interactive with an active or an inactive form of IL-4, the difference being that antigen-binding molecules to the active cytokine are more likely to recognise epitopes that are only present in the active conformation. Representative examples of such inhibitors include ligands or single-chain antibodies.

In some embodiments, the inhibitor of IL-4 function is an IL-4 trap

In some embodiments, the second agent and the third agent may comprise the same molecule. In specific embodiments, the second agent and the third agent comprise IL-4C118. IL-4C118 can bind to both the IL-13 receptor and IL-4 receptor preventing cellular signalling through these pathways.

2.3 Immune-Stimulating Agents

2.3.1 Antigens

The present invention contemplates the use in the compositions of the invention of an immune stimulator comprising any antigen that corresponds to at least a portion of a target antigen of interest for stimulating an immune response to the target antigen. The antigen that corresponds to at least a portion of the target antigen may be in soluble form (e.g., a peptide or polypeptide) when expressed.

Target antigens useful in the present invention are typically proteinaceous molecules, representative examples of which include polypeptides and peptides. Target antigens may be selected from endogenous antigens produced by a host or exogenous antigens that are foreign to the host. Suitable endogenous antigens include, but are not restricted to, cancer or tumor antigens. Non-limiting examples of cancer or tumor antigens include antigens from a cancer or tumor selected from ABL1 proto-oncogene, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, anal cancer, angiosarcoma, aplastic anemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, dermatofibrosarcoma protuberans, desmoplastic small round cell tumor, ductal carcinoma, endocrine cancers, endometrial cancer, ependymoma, oesophageal cancer, Ewing's Sarcoma, extra-hepatic bile duct cancer, eye cancer, melanoma, retinoblastoma, fallopian tube cancer, Fanconi anemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynecological cancers, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's cell histiocytosis, laryngeal cancer, leiomyosarcoma, leukemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid tumor of kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small-cell-lung-cancer (NSCLC), ocular cancers, esophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal tumours, pituitary cancer, polycythemia vera, prostate cancer, rare cancers and associated disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's macroglobulinemia, Wilms' tumor. In certain embodiments, the cancer or tumor relates to nasopharyngeal cancer. Illustrative examples of nasopharyngeal cancer antigens include EBNA-1, LMP-1, LMP-2, or a combination thereof. Other tumour-specific antigens include, but are not limited to: etv6, am1l, cyclophilin b (acute lymphoblastic leukemia); Ig-idiotype (B cell lymphoma); E-cadherin, α-catenin, β-catenin, γ-catenin, p120ctn (glioma); p21ras (bladder cancer); p21ras (biliary cancer); MUC family, HER2/neu, c-erbB-2 (breast cancer); p53, p21ras (cervical carcinoma); p21ras, HER2/neu, c-erbB-2, MUC family, Cripto-1protein, Pim-1 protein (colon carcinoma); Colorectal associated antigen (CRC)-CO17-1A/GA733, APC (colorectal cancer); carcinoembryonic antigen (CEA) (colorectal cancer; choriocarcinoma); cyclophilin b (epithelial cell cancer); HER2/neu, c-erbB-2, ga733 glycoprotein (gastric cancer); α-fetoprotein (hepatocellular cancer); Imp-1, EBNA-1 (Hodgkin's lymphoma); CEA, MAGE-3, NY-ESO-1 (lung cancer); cyclophilin b (lymphoid cell-derived leukemia); melanocyte differentiation antigen (e.g., gp100, MART, Melan-A/MART-1, TRP-1, Tyros, TRP2, MC1R, MUC1F, MUC1R or a combination thereof) and melanoma-specific antigens (e.g., BAGE, GAGE-1, gp100In4, MAGE-1 (e.g., GenBank Accession No. X54156 and AA494311), MAGE-3, MAGE4, PRAME, TRP2IN2, NYNSO1a, NYNSO1b, LAGE1, p97 melanoma antigen (e.g., GenBank Accession No. M12154) p5 protein, gp75, oncofetal antigen, GM2 and GD2 gangliosides, cdc27, p21ras, gp100^{Pmel117} or a combination thereof (melanoma); MUC family, p21ras (myeloma); HER2/neu, c-erbB-2 (non-small cell lung carcinoma); MUC family, HER2/neu, c-erbB-2, MAGE-A4, NY-ESO-1 (ovarian cancer); Prostate Specific Antigen (PSA) and its antigenic epitopes PSA-1, PSA-2, and PSA-3, PSMA, HER2/neu, c-erbB-2, ga733 glycoprotein (prostate cancer); HER2/neu, c-erbB-2 (renal cancer); viral products such as human papillomavirus proteins (squamous cell cancers of the cervix and esophagus); NY-ESO-1 (testicular cancer); and HTLV-1 epitopes (T cell leukemia).

Foreign or exogenous antigens are suitably selected from antigens of pathogenic organisms. Exemplary pathogenic organisms include, but are not limited to, viruses, bacteria, fungi, parasites, algae and protozoa and amoebae. Illustrative viruses include viruses responsible for diseases including, but not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Epstein-Barr virus and other herpesviruses such as papillomavirus, Ebola virus, influenza virus, Japanese encephalitis (e.g., GenBank Accession No. E07883), dengue (e.g., GenBank Accession No. M24444), hantavirus, Sendai virus, respiratory syncytial virus, othromyxoviruses, vesicular stomatitis virus, visna virus, cytomegalovirus and human immunodeficiency virus (HIV) (e.g., GenBank Accession No. U18552). Any suitable antigen derived from such viruses are useful in the practice of the present invention. For example, illustrative retroviral antigens derived from HIV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis, e.g., hepatitis A, B, and C, viral components such as hepatitis C viral RNA. Illustrative examples of influenza viral antigens include; but are not limited to, antigens such as hemagglutinin and neurarninidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of cytomegaloviral antigens include, but are not limited to, antigens such as envelope glycoprotein B and other cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as 9PI, gpII, and other varicella zoster viral antigen components. Non-limiting examples of Japanese encephalitis viral antigens include antigens such as proteins E, M-E, M-E-NS 1, NS 1, NS 1-NS2A, and other Japanese encephalitis viral antigen components. Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers, See Fundamental Virology, Second Edition, eds. Fields, B. N. and Knipe, D. M., 1991, Raven Press, New York, for additional examples of viral antigens.

Illustrative examples of fungi include Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselmei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus. Thus, representative fungal antigens that can be used in the compositions and methods of the present invention include, but are not limited to, candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Illustrative examples of bacteria include bacteria that are responsible for diseases including, but not restricted to, diphtheria (e.g., Corynebacterium diphtheria), pertussis (e.g., Bordetella pertussis, GenBank Accession No. M35274), tetanus (e.g., Clostridium tetani, GenBank Accession No. M64353), tuberculosis (e.g., Mycobacterium tuberculosis), bacterial pneumonias (e.g., Haemophilus influenzae.), cholera (e.g., Vibrio cholerae), anthrax (e.g., Bacillus anthracis), typhoid, plague, shigellosis (e.g., Shigella dysenteriae), botulism (e.g., Clostridium botulinum), salmonellosis (e.g., GenBank Accession No. L03833), peptic ulcers (e.g., Helicobacter pylori), Legionnaire's Disease, Lyme disease (e.g., GenBank Accession No. U59487), Other pathogenic bacteria include Escherichia coli, Clostridium perfringens, Pseudomonas aeruginosa, Staphylococcus aureus and Streptococcus pyogenes. Thus, bacterial antigens which can be used in the compositions and methods of the invention include, but are not limited to: pertussis bacterial antigens such as pertussis toxin, filamentous hemagglutinin, pertactin, F M2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components, streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components, pneumococcal bacterial antigens such as pneumolysin, pneumococcal capsular polysaccharides and other pnermiococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens.

Illustrative examples of protozoa include protozoa that are responsible for diseases including, but not limited to, malaria (e.g., GenBank Accession No. X53832), hookworm, onchocerciasis (e.g., GenBank Accession No. M27807), schistosomiasis (e.g., GenBank Accession No. LOS 198), toxoplasmosis, trypanosomiasis, leishmaniasis, giardiasis (GenBank Accession No. M33641), amoebiasis, filariasis (e.g., GenBank Accession No. J03266), borreliosis, and trichinosis. Thus, protozoal antigens which can be used in the compositions and methods of the invention include, but are not limited to: plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

The present invention also contemplates toxin components as antigens. Illustrative examples of toxins include, but are not restricted to, staphylococcal enterotoxins, toxic shock syndrome toxin; retroviral antigens (e.g., antigens derived from HIV), streptococcal antigens, staphylococcal enterotoxin-A (SEA), staphylococcal enterotoxin-B (SEB), staphylococcal enterotoxin₁₋₃ (SE₁₋₃), staphylococcal enterotoxin-D (SED), staphylococcal enterotoxin-E (SEE) as well as toxins derived from mycoplasma, mycobacterium, and herpes viruses.

Peptide antigens may be of any suitable size that can be utilised to stimulate or inhibit an immune response to a target antigen of interest. A number of factors can influence the choice of peptide size. For example, the size of a peptide can be chosen such that it includes, or corresponds to the size of, T cell epitopes and/or B cell epitopes, and their processing requirements. Practitioners in the art will recognise that class I-restricted T cell epitopes are typically between 8 and 10 amino acid residues in length and if placed next to unnatural flanking residues, such epitopes can generally require 2 to 3 natural flanking amino acid residues to ensure that they are efficiently processed and presented. Class II-restricted T cell epitopes usually range between 12 and 25 amino acid residues in length and may not require natural flanking residues for efficient proteolytic processing although it is believed that natural flanking residues may play a role. Another important feature of class II-restricted epitopes is that they generally contain a core of 9-10 amino acid residues in the middle which bind specifically to class II MHC molecules with flanking sequences either side of this core stabilising binding by associating with conserved structures on either side of class II MHC antigens in a sequence independent manner. Thus the functional region of class II-restricted epitopes is typically less than about 15 amino acid residues long. The size of linear B cell epitopes and the factors effecting their processing, like class H-restricted epitopes, are quite variable although such epitopes are frequently smaller in size than 15 amino acid residues. From the foregoing, it is advantageous, but not essential, that the size of the peptide is at least 6, 7, 8, 9, 10, 11, 12, 13, 14, 15, 20, 25, 30 amino acid residues. Suitably, the size of the peptide is no more than about 500, 200, 100, 80, 60, 50, 40 amino acid residues. In certain advantageous embodiments, the size of the peptide is sufficient for presentation by an antigen-presenting cell of a T cell and/or a B cell epitope contained within the peptide.

Criteria for identifying and selecting effective antigenic peptides (e.g., minimal peptide sequences capable of eliciting an immune response) can be found in the art. For example, Apostolopoulos et al. (2000, Curr. Opin. Mol. Ther. 2:29-36) discusses the strategy for identifying minimal antigenic peptide sequences based on an understanding of the three dimensional structure of an antigen-presenting molecule and its interaction with both an antigenic peptide and T-cell receptor. Shastri (1996, Curr. Opin. Immunol. 8:271-277) discloses how to distinguish rare peptides that serve to activate T cells from the thousands peptides normally bound to MHC molecules.

2.4 Compositions

Exemplary compositions of the present invention include vaccines or constructs, including but not limited to recombinant vaccines.

In some embodiments, the composition comprises a nucleic acid composition comprising: a first agent comprising a first polynucleotide sequence which encodes an immune stimulator that stimulates or otherwise enhances an immune response to the target antigen and which is operably linked to a regulatory polynucleotide, and a second agent comprising a second polynucleotide sequence which encodes an inhibitor of IL-13 function and which is operably linked to a regulatory polynucleotide. The regulatory polynucleotide may be the same or different.

In some embodiments, the first and second polynucleotides are located on the same construct (or expression vector). In other embodiments, the first and second polynucleotides are located on different constructs. Optionally, the construct(s) may further include a third polynucleotide that encodes an inhibitor of IL-4 function.

The regulatory polynucleotide suitably comprises transcriptional and/or translational control sequences, which will be compatible for expression in the cell or tissue type of interest. Typically, the transcriptional and translational regulatory control sequences include, but are not limited to, a promoter sequence, a 5′ non-coding region, a cis-regulatory region such as a functional binding site for transcriptional regulatory protein or translational regulatory protein, an upstream open reading frame, ribosomal-binding sequences, transcriptional start site, translational start site, and/or nucleotide sequence which encodes a leader sequence, termination codon, translational stop site and a 3′ non-translated region. Constitutive or inducible promoters as known in the art are contemplated by the invention. The promoters may be either naturally occurring promoters, or hybrid promoters that combine elements of more than one promoter. Promoter sequences contemplated by the present invention may be native to the organism of interest or may be derived from an alternative source, where the region is functional in the chosen organism. The choice of promoter will differ depending on the intended host. For example, promoters which could be used for expression in mammalian cells generally include the metallothionein promoter, which can be induced in response to heavy metals such as cadmium, the β-actin promoter as well as viral promoters such as the SV40 large T antigen promoter, human cytomegalovirus (CMV) immediate early (IE) promoter, rous sarcoma virus LTR promoter, adenovirus promoter, or a HPV promoter, particularly the HPV upstream regulatory region (URR) may also be used. All these promoters are well described and readily available in the art. Alternatively, the promoter may be lineage specific and, in this regard, epithelial-specific promoters are particularly desirable such as, but not limited to, promoters of the following genes transglutaminase type 1, involucrin, loricrin, SPR genes and filagrin as well as those of keratin genes (e.g., K10, K14, K5, K1).

The synthetic construct (or expression vector) may also comprise a 3′ non-translated sequence. A 3′ non-translated sequence refers to that portion of a gene comprising a DNA segment that contains a polyadenylation signal and any other regulatory signals capable of effecting mRNA processing or gene expression. The polyadenylation signal is characterised by effecting the addition of polyadenylic acid tracts to the 3′ end of the mRNA precursor. Polyadenylation signals are commonly recognised by the presence of homology to the canonical form 5′ AATAAA-3′ although variations are not uncommon. The 3′ non-translated regulatory DNA sequence preferably includes from about 50 to 1,000 nucleotide base pairs and may contain transcriptional and translational termination sequences in addition to a polyadenylation signal and any other regulatory signals capable of effecting mRNA processing or gene expression.

In some embodiments, the synthetic construct (or expression vector) further contains a screenable marker gene to permit identification of cells containing the synthetic construct. Screenable genes (e.g., lacZ, gfp, etc) are well known in the art and will be compatible for expression in a particular cell or tissue type.

It will be understood, however, that expression of protein-encoding polynucleotides in heterologous systems is now well known, and the present invention is not directed to or dependent on any particular vector, transcriptional control sequence or technique for its production. Rather, synthetic polynucleotides prepared according to the methods as set forth herein may be introduced into selected cells or tissues or into a precursors or progenitors thereof in any suitable manner in conjunction with any suitable synthetic construct or vector, and the synthetic polynucleotides may be expressed with known promoters in any conventional manner.

The synthetic constructs or vectors can be introduced into suitable host cells for expression using any of a number of non-viral or viral gene delivery vectors. For example, retroviruses (in particular, lentiviral vectors) provide a convenient platform for gene delivery systems. A coding sequence of interest (for example, a sequence useful for gene therapy applications) can be inserted into a gene delivery vector and packaged in retroviral particles using techniques known in the art. Recombinant virus can then be isolated and delivered to cells of the subject either in vivo or ex vivo.

In one illustrative embodiment, retroviruses provide a convenient and effective platform for gene delivery systems. A selected nucleotide sequence that encodes an antigen corresponding to the target antigen and a selected nucleotide sequence that encodes an inhibitor of IL-13 function (where the two selected nucleotide sequences can be part of the same sequence or separate) can be inserted into a construct or vector and packaged in retroviral particles using techniques known in the art. The construct or vector may also comprise a nucleotide sequence that encodes an inhibitor or IL-4 function, where this nucleotide sequence may be part of the nucleotide sequence that encodes an antigen corresponding to the target antigen or the nucleotide sequence that encodes an inhibitor of IL-13 function, or may be part of both the nucleotide sequence that encodes an antigen corresponding to the target antigen and the nucleotide sequence that encodes an inhibitor of IL-13 function, or may be separate. The recombinant virus can then be isolated and delivered to a subject. Several illustrative retroviral systems have been described examples of which include: U.S. Pat. No. 5,219,740; Miller and Rosman, 1989, Bio Techniques 7: 980-990; Miller, A. D., 1990, Human Gene Therapy 1: 5-14; Scarpa et al., 1991, Virology 180: 849-852; Burns et al., 1993, Proc. Natl. Acad. Sci. USA 90: 8033-8037; and Boris-Lawrie and Temin, 1993, Cur. Opin. Genet. Develop. 3: 102-109).

In addition, several illustrative adenovirus-based systems have also been described. Unlike retroviruses which integrate into the host genome, adenoviruses persist extrachromosomally thus minimising the risks associated with insertional mutagenesis (see, e.g., Haj-Ahmad and Graham, 1986, J. Virol. 57: 267-274; Bett et al., 1993, J. Virol. 67: 5911-5921; Mittereder et al., 1994, Human Gene Therapy 5: 717-729; Seth et al., 1994, J. Virol. 68: 933-940, ; Barr et al., 1994, Gene Therapy 1: 51-58; Berkner, K. L., 1988, Bio Techniques 6: 616-629; and Rich et al., 1993, Human Gene Therapy 4: 461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al., 1988, Molec. Cell. Biol. 8: 3988-3996; Vincent et al., 1990, Vaccines 90, Cold Spring Harbor Laboratory Press; Carter, B. J., 1992, Current Opinion in Biotechnology 3: 533-539; Muzyczka, N., 1992, Current Topics in Microbiol. and Immunol. 158: 97-129; Kotin, R. M., 1994, Human Gene Therapy 5: 793-801; Shelling and Smith, 1994, Gene Therapy 1: 165-169; and Zhou et al., 1994, J. Exp. Med. 179: 1867-1875.

Additional viral vectors useful for delivering the antigen-encoding polynucleotide and the IL-13 inhibitor-encoding polynucleotide (which can be the same polynucleotide or two separate polynucleotides), and optionally an IL-4 inhibitor-encoding polynucleotide (which can be the same polynucleotide as the antigen-encoding polynucleotide and/or the IL-13 inhibitor-encoding polynucleotide or separate) by gene transfer include those derived from the pox family of viruses, such as vaccinia virus and avian poxvirus. By way of example, vaccinia virus recombinants expressing the antigen-encoding polynucleotide and the IL-13 inhibitor-encoding polynucleotide, and optionally the IL-4 inhibitor-encoding polynucleotide, can be constructed as follows. The polynucleotides are first inserted into an appropriate vector so that it is adjacent to a vaccinia, promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the expression products of interest into the viral genome. The resulting TK⁽⁻⁾ recombinant can be selected by culturing the cells in the presence of 5-BrdU and picking viral plaques resistant thereto.

Alternatively, avipoxviruses, such as the fowlpox and canarypox viruses, can also be used to deliver the coding sequences of interest. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al., J. Biol. Chem. 268:6866-69, 1993; and Wagner et al., Proc. Natl. Acad. Sci. USA 89:6099-6103, 1992, can also be used for gene delivery under the invention.

In other illustrative embodiments, lentiviral vectors are employed to deliver an antigen-encoding polynucleotide into selected cells or tissues. Typically, these vectors comprise a 5′ lentiviral LTR, a tRNA binding site, a packaging signal, a promoter operably linked to one or more genes of interest, an origin of second strand DNA synthesis and a 3′ lentiviral LTR, wherein the lentiviral vector contains a nuclear transport element. The nuclear transport element may be located either upstream (5′) or downstream (3′) of a coding sequence of interest (for example, a synthetic Gag or Env expression cassette of the present invention). A wide variety of lentiviruses may be utilised within the context of the present invention, including for example, lentiviruses selected from the group consisting of HIV, HIV-1, HIV-2, FIV, BIV, EIAV, MVV, CAEV, and SW. Illustrative examples of lentiviral vectors are described in PCT Publication Nos. WO 00/66759, WO 00/00600, WO 99/24465, WO 98/51810, WO 99/51754, WO 99/31251, WO 99/30742, and WO 99/15641. Desirably, a third generation SIN lentivirus is used. Commercial suppliers of third generation SIN (self-inactivating) lentiviruses include Invitrogen (ViraPower Lentiviral Expression System). Detailed methods for construction, transfection, harvesting, and use of lentiviral vectors are given, for example, in the Invitrogen technical manual “ViraPower Lentiviral Expression System version B 050102 25-0501”, available at http://www.invitrogen.com/Content/Tech-Online/molecular_biology/manuals_p-ps/virapower_lentiviral_system_man.pdf. Lentiviral vectors have emerged as an efficient method for gene transfer. Improvements in biosafety characteristics have made these vectors suitable for use at biosafety level 2 (BL2). A number of safety features are incorporated into third generation SIN (self-inactivating) vectors. Deletion of the viral 3′ LTR U3 region results in a provirus that is unable to transcribe a full length viral RNA. In addition, a number of essential genes are provided in trans, yielding a viral stock that is capable of but a single round of infection and integration. Lentiviral vectors have several advantages, including: 1) pseudotyping of the vector using amphotropic envelope proteins allows them to infect virtually any cell type; 2) gene delivery to quiescent, post mitotic, differentiated cells, including neurones, has been demonstrated; 3) their low cellular toxicity is unique among transgene delivery systems; 4) viral integration into the genome permits long term transgene expression; 5) their packaging capacity (6-14 kb) is much larger than other retroviral, or adeno-associated viral vectors. In a recent demonstration of the capabilities of this system, lentiviral vectors expressing GFP were used to infect murine stem cells resulting in live progeny, germline transmission, and promoter-, and tissue-specific expression of the reporter (Ailles, L. E. and Naldini, L., HIV-1-Derived Lentiviral Vectors. In: Trono, D. (Ed.), Lentiviral Vectors, Springer-Verlag, Berlin, Heidelberg, New York, 2002, pp. 31-52). An example of the current generation vectors is outlined in FIG. 2 of a review by Lois et al. (Lois, C., Hong, E. J., Pease, S., Brown, E. J., and Baltimore, D., Germline transmission and tissue-specific expression of transgenes delivered by lentiviral vectors, Science, 295 (2002) 868-872).

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronisation with the host cell cycle. The manner in which the expression construct is delivered to a cell and where in the cell the polynucleotide remains is dependent on the type of expression construct employed.

In other embodiments, a polynucleotide is administered/delivered as “naked” DNA, for example as described in Ulmer et al., Science 259:1745-49, 1993 and reviewed by Cohen, Science 259:1691-92, 1993. The uptake of naked DNA may be increased by coating the DNA onto biodegradable beads, which are efficiently transported into the cells.

In still other embodiments, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

2.4.1 Immune-Stimulating Cell Embodiments

Antigen-Presenting Cells

The present invention also contemplates the use of antigen-presenting cells, which present an antigen corresponding to at least a portion of the target antigen, in the compositions of the present invention and which express or otherwise produce the inhibitor of IL-13 function. The antigen-presenting cells may also express or otherwise produce an inhibitor of IL-4 function. Such antigen-presenting cells include professional or facultative antigen-presenting cells. Professional antigen-presenting cells function physiologically to present antigen in a form that is recognised by specific T cell receptors so as to stimulate or anergise a T lymphocyte or B lymphocyte mediated immune response. Professional antigen-presenting cells not only process and present antigens in the context of the major histocompatibility complex (MHC), but also possess the additional immunoregulatory molecules required to complete T cell activation or induce a tolerogenic response. Professional antigen-presenting cells include, but are not limited to, macrophages, monocytes, B lymphocytes, cells of myeloid lineage, including monocytic-granulocytic-DC precursors, marginal zone Kupffer cells, microglia, T cells, Langerhans cells and dendritic cells including interdigitating dendritic cells and follicular dendritic cells. Non-professional or facultative antigen-presenting cells typically lack one or more of the immunoregulatory molecules required to complete T lymphocyte activation or anergy. Examples of non-professional or facultative antigen-presenting cells include, but are not limited to, activated T lymphocytes, eosinophils, keratinocytes, astrocytes, follicular cells, microglial cells, thymic cortical cells, endothelial cells, Schwann cells, retinal pigment epithelial cells, myoblasts, vascular smooth muscle cells, chondrocytes, enterocytes, thymocytes, kidney tubule cells and fibroblasts. In some embodiments, the antigen-presenting cell is selected from monocytes, macrophages, B lymphocytes, cells of myeloid lineage, dendritic cells or Langerhans cells. In certain advantageous embodiments, the antigen-presenting cell expresses CD11c and includes a dendritic cell or a Langerhans cell.

Antigen-presenting cells for stimulating an immune response to an antigen or group of antigens may be prepared according to any suitable method known to the skilled practitioner. Illustrative methods for preparing antigen-presenting cells for stimulating antigen-specific immune responses are described by Albert et al. (International Publication WO 99/42564), Takamizawa et al. (1997, J Immunol, 158(5): 2134-2142), Thomas and Lipsky (1994, J Immunol, 153(9):4016-4028), O'Doherty et al. (1994, Immunology, 82(3):487-93), Fearnley et al. (1997, Blood, 89(10): 3708-3716), Weissman et al. (1995, Proc Natl Acad Sci USA, 92(3):826-830), Freudenthal and Steinman (1990, Proc Natl Acad Sci USA, 87(19):7698-7702), Romani et al. (1996, J Immunol Methods, 196(2): 137-151), Reddy et al. (1997, Blood, 90(9):3640-3646), Thurnher et al. (1997, Exp Hematol, 25(3):232-237), Caux et al. (1996, J Exp Med, 184(2):695-706; 1996, Blood, 87(6):2376-85), Luft et al. (1998, Exp Hematol, 26(6):489-500; 1998, J Immunol, 161(4):1947-1953), Cella et al. (1999, J Exp Med, 189(5): 821-829;1997, Nature, 388(644):782-787; 1996, J Exp Med, 184(2):747-572), Ahonen et al. (1999, Cell Immunol, 197(1):62-72) and Piemonti et al. (1999, J Immunol, 162(11):6473-6481).

In some embodiments, the antigen-presenting cells are isolated from a host, treated and then re-introduced or reinfused into the host. Conveniently, antigen-presenting cells can be obtained from the host to be treated either by surgical resection, biopsy, blood sampling, or other suitable technique. Such cells are referred to herein as “autologous” cells. In other embodiments, the antigen-presenting cells or cell lines are prepared and/or cultured from a different source than the host. Such cells are referred to herein as “allogeneic” cells. Desirably, allogeneic antigen-presenting cells or cell lines will share major and/or minor histocompatibility antigens to potential recipients (also referred to herein as ‘generic’ antigen-presenting cells or cell lines). In certain advantageous embodiments of this type, the generic antigen-presenting cells or cell lines comprise major histocompatibility (MHC) class I antigens compatible with a high percentage of the population (i.e., at least 10, 20, 30, 40, 50, 60, 70, 75, 80, 85, 90, 92, 94 or 98%) that is susceptible or predisposed to a particular condition. Suitably, the generic antigen-presenting cells or cell lines naturally express an immunostimulatory molecule as described herein, especially an immunostimulatory membrane molecule, at levels sufficient to trigger an immune response, desirably a T lymphocyte immune response (e.g., a cytotoxic T lymphocyte immune response), in the intended host. In certain embodiments, the antigen-presenting cells or cell lines are highly susceptible to treatment with at least one IFN as described in International Publication No. WO 01/88097 (i.e., implied high level expression of class I HLA).

In some embodiments, antigen-presenting cells are made antigen-specific by a process including contacting or ‘pulsing’ the antigen-presenting cells with an antigen that corresponds to at least a portion of the target antigen for a time and under conditions sufficient to permit the antigen to be internalised by the antigen-presenting cells; and culturing the antigen-presenting cells so contacted for a time and under conditions sufficient for the antigen to be processed for presentation by the antigen-presenting cells. The pulsed cells can then be used to stimulate autologous or allogeneic T cells in vitro or in vivo. The amount of antigen to be placed in contact with antigen-presenting cells can be determined empirically by persons of skill in the art. Typically antigen-presenting cells are incubated with antigen for about 1 to 6 hr at 37° C. Usually, for purified antigens and peptides, 0.1-10 μg/mL is suitable for producing antigen-specific antigen-presenting cells. The antigen should be exposed to the antigen-presenting cells for a period of time sufficient for those cells to internalise the antigen. The time and dose of antigen necessary for the cells to internalise and present the processed antigen may be determined using pulse-chase protocols in which exposure to antigen is followed by a washout period and exposure to a read-out system e.g., antigen reactive T cells. Once the optimal time and dose necessary for cells to express processed antigen on their surface is determined, a protocol may be used to prepare cells and antigen for inducing tolerogenic responses. Those of skill in the art will recognise in this regard that the length of time necessary for an antigen-presenting cell to present an antigen may vary depending on the antigen or form of antigen employed, its dose, and the antigen-presenting cell employed, as well as the conditions under which antigen loading is undertaken. These parameters can be determined by the skilled artisan using routine procedures.

The delivery of exogenous antigen to an antigen-presenting cell can be enhanced by methods known to practitioners in the art. For example, several different strategies have been developed for delivery of exogenous antigen to the endogenous processing pathway of antigen-presenting cells, especially dendritic cells. These methods include insertion of antigen into pH-sensitive liposomes (Zhou and Huang, 1994, Immunomethods, 4:229-235), osmotic lysis of pinosomes after pinocytic uptake of soluble antigen (Moore et al., 1988, Cell, 54:777-785), coupling of antigens to potent adjuvants (Aichele et al., 1990, J. Exp. Med., 171: 1815-1820; Gao et al., 1991, J. Immunol., 147: 3268-3273; Schulz et al., 1991, Proc. Natl. Acad. Sci. USA, 88: 991-993; Kuzu et al., 1993, Euro. J. Immunol., 23: 1397-1400; and Jondal et al., 1996, Immunity 5: 295-302) and apoptotic cell delivery of antigen (Albert et al. 1998, Nature 392:86-89; Albert et al. 1998, Nature Med. 4:1321-1324; and in International Publications WO 99/42564 and WO 01/85207). Recombinant bacteria (e.g., E. coli) or transfected host mammalian cells may be pulsed onto dendritic cells (as particulate antigen, or apoptotic bodies respectively) for antigen delivery. Recombinant chimeric virus-like particles (VLPs) have also been used as vehicles for delivery of exogenous heterologous antigen to the MHC class I processing pathway of a dendritic cell line (Bachmann et al., 1996, Eur. J. Immunol., 26(11): 2595-2600).

Alternatively, or in addition, an antigen may be linked to, or otherwise associated with, a cytolysin to enhance the transfer of the antigen into the cytosol of an antigen-presenting cell of the invention for delivery to the MHC class I pathway. Exemplary cytolysins include saponin compounds such as saponin-containing Immune Stimulating Complexes (ISCOMs) (see e.g., Cox and Coulter, 1997, Vaccine 15(3): 248-256 and U.S. Pat. No. 6,352,697), phospholipases (see, e.g., Camilli et al., 1991, J. Exp. Med. 173: 751-754), pore-forming toxins (e.g., an α-toxin), natural cytolysins of gram-positive bacteria, such as listeriolysin O (LLO, e.g., Mengaud et al., 1988, Infect. Immun. 56: 766-772 and Portnoy et al., 1992, Infect. Immun. 60: 2710-2717), streptolysin O (SLO, e.g., Palmer et al., 1998, Biochemistry 37(8): 2378-2383) and perfringolysin O (PFO, e.g., Rossjohn et al., Cell 89(5): 685-692). Where the antigen-presenting cell is phagosomal, acid activated cytolysins may be advantageously used. For example, listeriolysin exhibits greater pore-forming ability at mildly acidic pH (the pH conditions within the phagosome), thereby facilitating delivery of vacuole (including phagosome and endosome) contents to the cytoplasm (see, e.g., Portnoy et al., Infect. Immun. 1992, 60: 2710-2717).

The cytolysin may be provided together with a pre-selected antigen in the form of a single composition or may be provided as a separate composition, for contacting the antigen-presenting cells. In one embodiment, the cytolysin is fused or otherwise linked to the antigen, wherein the fusion or linkage permits the delivery of the antigen to the cytosol of the target cell. In another embodiment, the cytolysin and antigen are provided in the form of a delivery vehicle such as, but not limited to, a liposome or a microbial delivery vehicle selected from virus, bacterium, or yeast. Suitably, when the delivery vehicle is a microbial delivery vehicle, the delivery vehicle is non-virulent. In a preferred embodiment of this type, the delivery vehicle is a non-virulent bacterium, as for example described by Portnoy et al. in U.S. Pat. No. 6,287,556, comprising a first polynucleotide encoding a non-secreted functional cytolysin operably linked to a regulatory polynucleotide which expresses the cytolysin in the bacterium, and a second polynucleotide encoding one or more pre-selected antigens. Non-secreted cytolysins may be provided by various mechanisms, e.g., absence of a functional signal sequence, a secretion incompetent microbe, such as microbes having genetic lesions (e.g., a functional signal sequence mutation), or poisoned microbes, etc. A wide variety of nonvirulent, non-pathogenic bacteria may be used; preferred microbes are relatively well characterised strains, particularly laboratory strains of E. coli, such as MC4100, MC1061, DH5α, etc. Other bacteria that can be engineered for the invention include well-characterised, nonvirulent, non-pathogenic strains of Listeria monocytogenes, Shigella flexneri, mycobacterium, Salmonella, Bacillus subtilis, etc. In particular embodiments, the bacteria are attenuated to be non-replicative, non-integrative into the host cell genome, and/or non-motile inter- or intra-cellularly.

In some other embodiments, in order to enhance the class I presentation of the antigen, the antigen is modified to comprise an intracellular degradation signal or degron. The degron is suitably a ubiquitin-mediated degradation signal selected from a destabilising amino acid at the amino-terminus of an antigen, a ubiquitin acceptor, a ubiquitin or combination thereof.

Thus, in one embodiment, the antigen is modified to include a destabilising amino acid at its amino-terminus so that the protein so modified is subject to the N-end rule pathway as disclosed, for example, by Bachmair et al., in U.S. Pat. No. 5,093,242 and by Varshaysky et al., in U.S. Pat. No. 5,122,463. In a preferred embodiment of this type, the destabilising amino acid is selected from isoleucine and glutamic acid, more preferably from histidine tyrosine and glutamine, and even more preferably from aspartic acid, asparagine, phenylalanine, leucine, tryptophan and lysine. In an especially preferred embodiment, the destabilising amino acid is arginine.

Modification or design of the amino-terminus of a protein can also be accomplished at the genetic level. Conventional techniques of site-directed mutagenesis for addition or substitution of appropriate codons to the 5′ end of an isolated or synthesised antigen-encoding polynucleotide can be employed to provide a desired amino-terminal structure for the encoded protein. For example, so that the protein expressed has the desired amino acid at its amino-terminus the appropriate codon for a destabilising amino acid can be inserted or built into the amino-terminus of the protein-encoding sequence. Where necessary, a nucleic acid sequence encoding the amino-terminal region of a protein can be modified to introduce one or more lysine residues in an appropriate context, which act as a ubiquitin acceptor as described in more detail below. This can be achieved most conveniently by employing DNA constructs encoding “universal destabilising segments”. A universal destabilising segment comprises a nucleic acid construct which encodes a polypeptide structure, preferably segmentally mobile, containing one or more lysine residues, the codons for lysine residues being positioned within the construct such that when the construct is inserted into the coding sequence of the antigen-encoding polynucleotide, the lysine residues are sufficiently spatially proximate to the amino-terminus of the encoded protein to serve as the second determinant of the complete amino-terminal degradation signal. The insertion of such constructs into the 5′ portion of an antigen-encoding polynucleotide would provide the encoded protein with a lysine residue (or residues) in an appropriate context for destabilisation.

The codon for the amino-terminal amino acid of the protein of interest can be made to encode the desired amino acid by, for example, site-directed mutagenesis techniques currently standard in the field. Suitable mutagenesis methods are described for example in the relevant sections of Ausubel, et al. (supra) and of Sambrook, et al., (supra). Alternatively, suitable methods for altering DNA are set forth, for example, in U.S. Pat. Nos. 4,184,917, 4,321,365 and 4,351,901, which are incorporated herein by reference. Instead of in vitro mutagenesis, the synthetic polynucleotide can be synthesised de novo using readily available machinery. Sequential synthesis of DNA is described, for example, in U.S. Pat. No. 4,293,652. However, it should be noted that the present invention is not dependent on, and not directed to, any one particular technique for constructing a polynucleotide encoding a modified antigen as described herein.

If the antigen-encoding polynucleotide is a synthetic or recombinant polynucleotide the appropriate 5′ codon can be built-in during the synthetic process. Alternatively, nucleotides for a specific codon can be added to the 5′ end of an isolated or synthesised polynucleotide by ligation of an appropriate nucleic acid sequence to the 5′ (amino-terminus-encoding) end of the polynucleotide. Nucleic acid inserts encoding appropriately located lysine residues (such as the “universal destabilising segments” described above) can suitably be inserted into the 5′ region to provide for the second determinant of the complete amino-terminal degradation.

In a preferred embodiment, the modified antigen, which comprises a destabilising amino acid at its amino terminus, is fused or otherwise conjugated to a masking entity, which masks said amino terminus so that when unmasked the antigen will exhibit the desired rate of intracellular proteolytic degradation. Suitably, the masking entity is a masking protein sequence. The fusion protein is designed so that the masking protein sequence fused to the amino-terminus of the protein of interest is susceptible to specific cleavage at the junction between the two. Removal of the protein sequence thus unmasks the amino-terminus of the protein of interest and the half-life of the released protein is thus governed by the predesigned amino-terminus. The fusion protein can be designed for specific cleavage in vivo, for example, by a host cell endoprotease or for specific cleavage in an in vitro system where it can be cleaved after isolation from a producer cell (which lacks the capability to cleave the fusion protein). Thus, in a preferred embodiment, the masking protein sequence is cleavable by an endoprotease, which is preferably an endogenous endoprotease of a mammalian cell. Suitable endoproteases include, but are not restricted to, serine endoproteases (e.g., subtilisins and furins) as described, for example, by Creemers, et al. (1998, Semin. Cell Dev. Biol. 9 (1): 3-10), proteasomal endopeptidases as described, for example, by Zwickl, et al. (2000, Curr. Opin. Struct. Biol. 10 (2): 242-250), proteases relating to the MHC class I processing pathway as described, for example, by Stolze et al. (2000, Nat. Immunol. 1 413-418) and signal peptidases as described, for example, by Dalbey, et al. (1997, Protein Sci. 6 (6): 1129-1138). In a preferred embodiment of this type, the masking protein sequence comprises a signal peptide sequence. Suitable signal peptides sequences are described, for example, by Nothwehr et al. (1990, Bioessays 12 (10): 479-484), Izard, et al. (1994, Mol. Microbiol. 13 (5): 765-773), Menne, et al. (2000, Bioinformatics. 16 (8): 741-742) and Ladunga (2000, Curr. Opin. Biotechnol. 11 (1): 13-18). Suitably, an endoprotease cleavage site is interposed between the masking protein sequence and the antigen.

A modified antigen with an attached masking sequence may be conveniently prepared by fusing a nucleic acid sequence encoding a masking protein sequence upstream of another nucleic acid sequence encoding an antigen, which corresponds to the target antigen of interest and which includes a destabilising amino acid at its amino-terminus. The codon for the amino-terminal amino acid of the antigen of interest is suitably located immediately adjacent to the 3′ end of the masking protein-encoding nucleic acid sequence.

In another embodiment, the antigen is modified to include, or is otherwise associated with, an ubiquitin acceptor which is a molecule that preferably contains at least one residue appropriately positioned from the N-terminal of the antigen as to be able to be bound by ubiquitin molecules. Such residues preferentially have an epsilon amino group such as lysine. Physical analysis demonstrates that multiple lysine residues function as ubiquitin acceptor sites (King et al., 1996, Mol. Biol. Cell 7: 1343-1357; King et al., 1996, Science 274: 1652-1659). Examples of other ubiquitin acceptors include lacI or Sindis virus RNA polymerase. Ubiquitination at the N-terminal of the protein specifically targets the protein for degradation via the ubiquitin-proteosome pathway.

Other protein processing signals that destabilise an antigen of interest and allow for enhanced intracellular degradation are contemplated in the present invention. These other methods may not necessarily be mediated by the ubiquitin pathway, but may otherwise permit degradation of proteins in the cytoplasm via proteosomes. For example, the present invention contemplates the use of other intracellular processing signals which govern the rate(s) of intracellular protein degradation including, but not limited to, those described by Bohley et al. (1996, Biol. Chem. Hoppe. Seyler 377: 425-435). Such processing signals include those that allow for phosphorylation of the target protein (Yaglom et al., 1996, Mol. Cell Biol. 16: 3679-3684; Yaglom et al., 1995, Mol. Cell Biol. 15: 731-741). Also contemplated by the present invention are modification of an parent antigen that allow for post-translational arginylation (Ferber et al. 1987, Nature 326: 808-811; Bohley et al., 1991, Biomed. Biochim. Acta 50: 343-346) of the protein which can enhance its rate(s) of intracellular degradation. The present invention also contemplates the use of certain structural features of proteins that can influence higher rates of intracellular protein turn-over, including protein surface hydrophobicity, clusters of hydrophobic residues within the protein (Sadis et al., 1995, Mol. Cell Biol. 15: 4086-4094), certain hydrophobic pentapeptide motifs at the protein's carboxy-terminus (C-terminus) (e.g., ARINV), as found on the C-terminus of ornithine decarboxylase (Ghoda et al., 1992, Mol. Cell Biol. 12: 2178-2185; Li, et al., 1994, Mol. Cell Biol. 14: 87-92), or AANDENYALAA, as found in C-terminal tags of aberrant polypeptides (Keiler et al., 1996, Science 271: 990-993,) or PEST regions (regions rich in proline (P), glutamic acid (E), serine (S), and threonine (T), which are optionally flanked by amino acids comprising electropositive side chains (Rogers et al. 1986, Science 234 (4774): 364-368; 1988, J. Biol. Chem. 263: 19833-19842). Moreover, certain motifs have been identified in proteins that appear necessary and possibly sufficient for achieving rapid intracellular degradation. Such motifs include RXALGXIXN region (where X=any amino acid) in cyclins (Glotzer et al., 1991, Nature 349: 132-138) and the KTKRNYSARD motif in isocitrate lyase (Ordiz et al., 1996, FEBS Lett. 385: 43-46).

The present invention also contemplates enhanced cellular degradation of a parent antigen which may occur by the incorporation into that antigen known protease cleavage sites. For example amyloid beta-protein can be cleaved by beta- and gamma-secretase (Iizuka et al., 1996, Biochem. Biophys. Res. Commun. 218: 238-242) and the two-chain vitamin K-dependent coagulation factor X can be cleaved by calcium-dependent endoprotease(s) in liver (Wallin et al., 1994, Thromb. Res. 73: 395-403).

In yet another embodiment, the parent antigen is conjugated to a ubiquitin or a biologically active fragment thereof, to produce a modified antigen whose rate of intracellular proteolytic degradation is increased, enhanced or otherwise elevated relative to the parent antigen. In a preferred embodiment of this type, the ubiquitin or biologically active fragment is fused, or otherwise conjugated, to the antigen. Suitably, the ubiquitin is of mammalian origin, more preferably of human or other primate origin.

In one embodiment, the ubiquitin-antigen fusion protein is suitably produced by covalently attaching an antigen corresponding to the target antigen to a ubiquitin or a biologically active fragment thereof. Covalent attachment may be effected by any suitable means known to persons of skill in the art. For example, protein conjugates may be prepared by linking proteins together using bifunctional reagents. The bifunctional reagents can be homobifunctional or heterobifunctional.

Homobifunctional reagents are molecules with at least two identical functional groups. The functional groups of the reagent generally react with one of the functional groups on a protein, typically an amino group. Examples of homobifunctional reagents include glutaraldehyde and diimidates. An example of the use of glutaraldehyde as a cross-linking agent is described by Poznansky et al. (1984, Science, 223: 1304-1306). The use of diimidates as a cross-linking agent is described for example by Wang, et al. (1977, Biochemistry, 16: 2937-2941). Although it is possible to use homobifunctional reagents for the purpose of forming a modified antigen according to the invention, skilled practitioners in the art will appreciate that it is difficult to attach different proteins in an ordered fashion with these reagents. In this regard, in attempting to link a first protein with a second protein by means of a homobifunctional reagent, one cannot prevent the linking of the first protein to each other and of the second to each other. Heterobifunctional crosslinking reagents are, therefore, preferred because one can control the sequence of reactions, and combine proteins at will. Heterobifunctional reagents thus provide a more sophisticated method for linking two proteins. These reagents require one of the molecules to be joined, hereafter called Partner B, to possess a reactive group not found on the other, hereafter called Partner A, or else require that one of the two functional groups be blocked or otherwise greatly reduced in reactivity while the other group is reacted with Partner A. In a typical two-step process for forming heteroconjugates, Partner A is reacted with the heterobifunctional reagent to form a derivatised Partner A molecule. If the unreacted functional group of the crosslinker is blocked, it is then deprotected. After deprotecting, Partner B is coupled to derivatised Partner A to form the conjugate. Primary amino groups on Partner A are reacted with an activated carboxylate or imidate group on the crosslinker in the derivatisation step. A reactive thiol or a blocked and activated thiol at the other end of the crosslinker is reacted with an electrophilic group or with a reactive thiol, respectively, on Partner B. When the crosslinker possesses a reactive thiol, the electrophile on Partner B preferably will be a blocked and activated thiol, a maleimide, or a halomethylene carbonyl (e.g., bromoacetyl or iodoacetyl) group. Because biological macromolecules do not naturally contain such electrophiles, they must be added to Partner B by a separate derivatisation reaction. When the crosslinker possesses a blocked and activated thiol, the thiol on Partner B with which it reacts may be native to Partner B.

An example of a heterobifunctional reagent is N-succinimidyl 3-(2-pyridyldithio)propionate (SPDP) (see for example Carlsson et al., 1978, Biochem. J., 173: 723-737). Other heterobifunctional reagents for linking proteins include for example succinimidyl 4-(N-maleimidomethyl)cyclohexane-1-carboxylate (SMCC) (Yoshitake et al., 1979, Eur. J. Biochem, 101: 395-399), 2-iminothiolane (IT) (Jue et al., 1978, Biochemistry, 17: 5399-5406), and S-acetyl mercaptosuccinic anhydride (SAMSA) (Klotz and Heiney, 1962, Arch. Biochem. Biophys., 96: 605-612). All three react preferentially with primary amines (e.g., lysine side chains) to form an amide or amidine group which links a thiol to the derivatised molecule (e.g., a heterologous antigen) via a connecting short spacer arm, one to three carbon atoms long. Examples of heterobifunctional reagents comprising reactive groups having a double bond that reacts with a thiol group include SMCC mentioned above, succinimidyl m-maleimidobenzoate, succinimidyl 3-(maleimido)propionate, sulfosuccinimidyl 4-(p-maleimidophenyl)butyrate, sulfosuccinimidyl 4-(N-maleimidomethylcyclohexane-1-carboxylate and maleimidobenzoyl-N-hydroxysuccinimide ester (MBS). In a preferred embodiment, MBS is used to produce the conjugate. Other heterobifunctional reagents for forming conjugates of two proteins are described for example by Rodwell et al. in U.S. Pat. No. 4,671,958 and by Moreland et al. in U.S. Pat. No. 5,241,078.

In an alternate embodiment, a ubiquitin-antigen fusion protein is suitably expressed by a synthetic chimeric polynucleotide comprising a first nucleic acid sequence, which encodes an antigen corresponding to the target antigen, and which is linked downstream of, and in reading frame with, a second nucleic acid sequence encoding a ubiquitin or biologically active fragment thereof. In a preferred embodiment of this type, the second polynucleotide comprises a first nucleic acid sequence, which encodes an antigen corresponding to the target antigen, and which is linked immediately adjacent to, downstream of, and in reading frame with, a second nucleic acid sequence encoding a ubiquitin or biologically active fragment thereof. In another embodiment, the second polynucleotide comprises a first nucleic acid sequence, which encodes an antigen corresponding to the target antigen, and which is linked upstream of, and in reading frame with, a second nucleic acid sequence encoding a ubiquitin or biologically active fragment thereof. In yet another embodiment of this type, the second polynucleotide comprises a first nucleic acid sequence, which encodes an antigen corresponding to the target antigen, and which is linked immediately adjacent to, upstream of, and in reading frame with, a second nucleic acid sequence encoding a ubiquitin or biologically active fragment thereof.

The delivery vehicles described above can be used to deliver one or more antigens to virtually any antigen-presenting cell capable of endocytosis of the subject vehicle, including phagocytic and non-phagocytic antigen-presenting cells. In embodiments when the delivery vehicle is a microbe, the subject methods generally require microbial uptake by the target cell and subsequent lysis within the antigen-presenting cell vacuole (including phagosomes and endosomes).

In other embodiments, the antigen is produced inside the antigen-presenting cell by introduction of a suitable expression vector as for example described above. The antigen-encoding portion of the expression vector may comprise a naturally-occurring sequence or a variant thereof, which has been engineered using recombinant techniques. In one example of a variant, the codon composition of an antigen-encoding polynucleotide is modified to permit enhanced expression of the antigen in a target cell or tissue of choice using methods as set forth in detail in International Publications WO 99/02694 and WO 00/42215. Briefly, these methods are based on the observation that translational efficiencies of different codons vary between different cells or tissues and that these differences can be exploited, together with codon composition of a gene, to regulate expression of a protein in a particular cell or tissue type. Thus, for the construction of codon-optimised polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5, 10, 15, 20, 25, 30%, more preferably 35, 40, 50, 60, 70% or more of the existing codons of a parent polynucleotide.

The expression vector for introduction into the antigen-presenting cell will be compatible therewith such that the antigen-encoding polynucleotide is expressible by the cell. For example, expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000, Curr. Opin. Biotechnol. 11(2):205-208), Vigna and Naldini (2000, J. Gene Med. 2(5):308-316), Kay, et al. (2001, Nat. Med. 7(1):33-40), Athanasopoulos, et al. (2000, Int. J. Mol. Med. 6(4):363-375) and Walther and Stein (2000, Drugs 60(2):249-271). The expression vector is introduced into the antigen-presenting cell by any suitable means which will be dependent on the particular choice of expression vector and antigen-presenting cell employed. Such means of introduction are well-known to those skilled in the art. For example, introduction can be effected by use of contacting (e.g., in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g., liposomes. Such liposomes are commercially available (e.g., Lipofectin®, Lipofectamine™, and the like, supplied by Life Technologies, Gibco BRL, Gaithersburg, Md.). It will be understood by persons of skill in the art that the techniques for assembling and expressing antigen-encoding nucleic acid molecules, immunoregulatory molecules and/or cytokines as described herein e.g., synthesis of oligonucleotides, nucleic acid amplification techniques, transforming cells, constructing vectors, expressions system and the like and transducing or otherwise introducing nucleic acid molecules into cells are well established in the art, and most practitioners are familiar with the standard resource materials for specific conditions and procedures.

In some embodiments, the antigen-specific antigen-presenting cells are obtained by isolating antigen-presenting cells or their precursors from a cell population or tissue to which modification of an immune response is desired. Typically, some of the isolated antigen-presenting cells or precursors will constitutively present antigens or have taken up such antigen in vivo that are targets or potential targets of an immune response for which stimulation or inhibition of an immune response is desired. In this instance, the delivery of exogenous antigen is not essential. Alternatively, cells may be derived from biopsies of healthy or diseased tissues, lysed or rendered apoptotic and the pulsed onto antigen-presenting cells (e.g., dendritic cells). In certain embodiments of this type, the antigen-presenting cells represent cancer or tumor cells to which an antigen-specific immune response is required. Illustrative examples of cancers or tumor cells include cells of sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; myelomonocytic, monocytic and erythroleukemia); chronic leukemia (chronic myelocytic (granulocyte) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In certain embodiments, the cancer or tumor cells are selected from the group consisting of melanoma cells and mammary carcinoma cells.

In some of the above embodiments, the cancer or tumor cells will constitute facultative or non-professional antigen-presenting cells, and may in some instances require further modification to enhance their antigen-presenting functions. In these instances, the antigen-presenting cells are further modified to express one or more immunoregulatory molecules, which include any molecules occurring naturally in animals that may regulate or directly influence immune responses including: proteins involved in antigen processing and presentation such as TAP1/TAP2 transporter proteins, proteosome molecules such as LMP2 and LMP7, heat shock proteins such as gp96, HSP70 and HSP90, and major histocompatibility complex (MHC) or human leukocyte antigen (HLA) molecules; factors that provide co-stimulation signals for T cell activation such as B7 and CD40; factors that provide co-inhibitory signals for direct killing of T cells or induction of T lymphocyte or B lymphocyte anergy or stimulation of T regulatory cell (Treg) generation such as OX-2, programmed death-1 ligand (PD-IL); accessory molecules such as CD83; chemokines; lymphokines and cytokines such as IFN s α, β and γ, interleukins (e.g., IL-2, IL-7, IL-12, IL-15, IL-22, etc.), factors stimulating cell growth (e.g., GM-SCF) and other factors (e.g., tumor necrosis factors (TNFs), DC-SIGN, MIP1α, MIP1β and transforming growth factor-β (TGF-β). In certain advantageous embodiments, the immunoregulatory molecules are selected from a B7 molecule (e.g., B7-1, B7-2 or B7-3) and an ICAM molecule (e.g., ICAM-1 and ICAM-2).

Instead of recombinantly expressing immunoregulatory molecules, antigen-presenting cells expressing the desired immunostimulatory molecule(s) may be isolated or selected from a heterogeneous population of cells. Any method of isolation/selection is contemplated by the present invention, examples of which are known to those of skill in the art. For instance, one can take advantage of one or more particular characteristics of a cell to specifically isolate that cell from a heterogeneous population. Such characteristics include, but are not limited to, anatomical location of a cell, cell density, cell size, cell morphology, cellular metabolic activity, cell uptake of ions such as Ca²⁺, K⁺, and H⁺ ions, cell uptake of compounds such as stains, markers expressed on the cell surface, protein fluorescence, and membrane potential. Suitable methods that can be used in this regard include surgical removal of tissue, flow cytometry techniques such as fluorescence-activated cell sorting (FACS), immunoaffinity separation (e.g., magnetic bead separation such as Dynabead™ separation), density separation (e.g., metrizamide, Percoll™, or Ficoll™ gradient centrifugation), and cell-type specific density separation. Desirably, the cells are isolated by flow cytometry or by immunoaffinity separation using an antigen-binding molecule that is immuno-interactive with the immunoregulatory molecule.

Alternatively, the immunoregulatory molecule can be provided to the antigen-presenting cells in soluble form. In some embodiments of this type, the immunoregulatory molecule is a B7 molecule that lacks a functional transmembrane domain (e.g., that comprises a B7 extracellular domain), non-limiting examples of which are described by McHugh et al. (1998, Clin. Immunol. Immunopathol. 87(1):50-59), Faas et al. (2000, J. Immunol. 164(12):6340-6348) and Jeannin et al. (2000, Immunity 13(3):303-312). In other embodiments of this type, the immunostimulatory protein is a B7 derivative including, but not limited to, a chimeric or fusion protein comprising a B7 molecule, or biologically active fragment thereof, or variant or derivative of these, linked together with an antigen binding molecule such as an immunoglobulin molecule or biologically active fragment thereof. For example, a polynucleotide encoding the amino acid sequence corresponding to the extracellular domain of the B7-1 molecule, containing amino acids from about position 1 to about position 215, is joined to a polynucleotide encoding the amino acid sequences corresponding to the hinge, CH2 and CH3 regions of human Ig Cγ1, using PCR, to form a construct that is expressed as a B7Ig fusion protein. DNA encoding the amino acid sequence corresponding to a B7Ig fusion protein has been deposited with the American Type culture Collection (ATCC) in Rockville, Md., under the Budapest Treaty on May 31, 1991 and accorded accession number 68627. Techniques for making and assembling such B7 derivatives are disclosed for example by Linsley et al. (U.S. Pat. No. 5,580,756). Reference also may be made to Sturmhoefel et al. (1999, Cancer Res. 59: 4964-4972) who disclose fusion proteins comprising the extracellular region of B7-1 or B7-2 fused in frame to the Fc portion of IgG2a.

The half-life of a soluble immunoregulatory molecule may be prolonged by any suitable procedure if desired. Preferably, such molecules are chemically modified with polyethylene glycol (PEG), including monomethoxy-polyethylene glycol, as for example disclosed by Chapman et al. (1999, Nature Biotechnology 17: 780-783).

Alternatively, or in addition, the antigen-presenting cells are cultured in the presence of at least one IFN for a time and under conditions sufficient to enhance the antigen presenting function of the cells and washing the cells to remove the IFN(s). In certain advantageous embodiments of this type, the step of culturing may comprise contacting the cells with at least one type I IFN and/or a type II IFN. The at least one type I IFN is suitably selected from the group consisting of an IFN-α, an IFN-β, a biologically active fragment of an IFN-α, a biologically active fragment of an IFN-β, a variant of an IFN-α, a variant of an IFN-β, a variant of a said biologically active fragment, a derivative of an IFN-α, a derivative of an IFN-β, a derivative of a said biologically active fragment, a derivative of a said variant, an analogue of IFN-α and an analogue of IFN-β. Typically, the type II IFN is selected from the group consisting of an IFN-γ, a biologically active fragment of an IFN-γ, a variant of an IFN-γ, a variant of said biologically active fragment, a derivative of an IFN-γ, a derivative of said biologically active fragment, a derivative of said variant and an analogue of an IFN-γ. Exemplary methods and conditions for enhancing the antigen-presenting functions of antigen-presenting cells using IFN treatment are described in International Publication No. WO 2001/88097.

In some embodiments, the antigen-presenting cells (e.g., cancer cells) or cell lines are suitably rendered inactive to prevent further proliferation once administered to the subject. Any physical, chemical, or biological means of inactivation may be used, including but not limited to irradiation (generally with at least about 5,000 cGy, usually at least about 10,000 cGy, typically at least about 20,000 cGy); or treatment with mitomycin-C (usually at least 10 μg/mL; more usually at least about 50 μg/mL).

The antigen-presenting cells may be obtained or prepared to contain and/or express one or more antigens by any number of means, such that the antigen(s) or processed form(s) thereof, is (are) presented by those cells for potential modulation of other immune cells, including T lymphocytes and B lymphocytes, and particularly for producing T lymphocytes and B lymphocytes that are primed to respond to a specified antigen or group of antigens.

The present invention also contemplates co-introducing an agent that comprises an inhibitor of IL-13 function, and/or an inhibitor of IL-4 function into an antigen-presenting cell or antigen-presenting cell precursor so that the antigen-present cell co-expresses or co-presents both the antigen and the inhibitor of IL-13 function and/or the inhibitor of IL-4 function.

The agents of the present invention may be encapsulated, adsorbed to, or associated with, particulate carriers. Such carriers can be used to selectively introduce the agents to cells of the immune system. The particles can be taken up by professional antigen presenting cells such as macrophages and dendritic cells, and/or can enhance antigen presentation through other mechanisms such as stimulation of cytokine release. Examples of particulate carriers include those derived from polymethyl methacrylate polymers, as well as microparticles derived from poly(lactides) and poly(lactide-co-glycolides), known as PLG. See, e.g., Jeffery et al., 1993, Pharm. Res. 10:362-368; McGee J. P., et al., 1997, J Microencapsul. 14(2):197-210; O'Hagan D. T., et al., 1993, Vaccine 11(2):149-54.

Furthermore, other particulate systems and polymers can be used for the in vivo delivery of the agents of the present invention. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods. See, e.g., Feigner, P. L., Advanced Drug Delivery Reviews (1990) 5:163-187, for a review of delivery systems useful for gene transfer. Peptoids (Zuckerman, R. N., et al., U.S. Pat. No. 5,831,005, issued Nov. 3, 1998) may also be used for delivery of a construct of the present invention.

Additionally, biolistic delivery systems employing particulate carriers such as gold and tungsten, are especially useful for delivering agents that are in nucleic acid form (e.g., constructs of the present invention). The particles are coated with the synthetic expression cassette(s) to be delivered and accelerated to high velocity, generally under a reduced atmosphere, using a gun powder discharge from a “gene gun.” For a description of such techniques, and apparatuses useful therefor, see, e.g., U.S. Pat. Nos. 4,945,050; 5,036,006; 5,100,792; 5,179,022; 5,371,015; and 5,478,744. In illustrative examples, gas-driven particle acceleration can be achieved with devices such as those manufactured by PowderMed Pharmaceuticals PLC (Oxford, UK) and PowderMed Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest. Other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

Alternatively, micro-cannula- and microneedle-based devices (such as those being developed by Becton Dickinson and others) can be used to administer nucleic acid constructs of the invention. Illustrative devices of this type are described in EP 1 092 444 A1, and U.S. application Ser. No. 606,909, filed Jun. 29, 2000. Standard steel cannula can also be used for intra-dermal delivery using devices and methods as described in U.S. Ser. No. 417,671, filed Oct. 14, 1999. These methods and devices include the delivery of substances through narrow gauge (about 30 G) “micro-cannula” with limited depth of penetration, as defined by the total length of the cannula or the total length of the cannula that is exposed beyond a depth-limiting feature. It is within the scope of the present invention that targeted delivery of substances including nucleic acid constructs can be achieved either through a single microcannula or an array of microcannula (or “microneedles”), for example 3-6 microneedles mounted on an injection device that may include or be attached to a reservoir in which the substance to be administered is contained.

2.5 Ancillary Components

In some embodiments the composition further comprises one or more cytokines, which are suitably selected from flt3, SCF, IL-3, IL-6, GM-CSF, G-CSF, TNF-α, TNF-β, LT-β, IL-2, IL-7, IL-9, IL-15, IL-5, IL-1α, IL-1β, IFN-7, IL-17, IL-16, IL-18, HGF, IL-11, MSP, FasL, TRAIL, TRANCE, LIGHT, TWEAK, CD27L, CD30L, CD40L, APRIL, TALL-1, 4-1BBL, OX40L, GITRL, IGF-I, IGF-II, HGF, MSP, FGF-a, FGF-b, FGF-3-19, NGF, BDNF, NTs, Tpo, Epo, Ang1-4, PDGF-AA, PDGF-BB, VEGF-A, VEGF-B, VEGF-C, VEGF-D, PIGF, EGF, TGF-α, AR, BTC, HRGs, HB-EGF, SMDF, OB, CT-1, CNTF, OSM, SCF, Flt-3L, M-CSF, MK and PTN or their functional, recombinant or chemical equivalents or homologues thereof. Preferably the cytokine is selected from the group consisting of IL-12, IL-3, IL-5, TNF, GMCSF, and IFN-γ.

### 3. Cell Based Therapy or Prophylaxis

In accordance with the present invention, an inhibitor of IL-13 function, as described for example in Section 2.1, can be administered to a patient, together with antigen-presenting cells as described in Section 2.3.2 for priming or boosting an immune response. Optionally, the patient may also be administered with an inhibitor of IL-4 function, as described for example in Section 2.2. These cell based compositions are useful, therefore, for treating or preventing a disease or condition that is associated with the presence or aberrant expression of a target antigen. The cells of the invention can be introduced into a patient by any means (e.g., injection), which produces the desired immune response to an antigen or group of antigens. The cells may be derived from the patient (i.e., autologous cells) or from an individual or individuals who are MHC matched or mismatched (i.e., allogeneic) with the patient. Typically, autologous cells are injected back into the patient from whom the source cells were obtained. The injection site may be mucosal, subcutaneous, intraperitoneal, intramuscular, intradermal, or intravenous. The cells may be administered to a patient to provide protective immunity or to a patient already suffering from a disease or condition or who is predisposed to a disease or condition in sufficient number to treat or prevent or alleviate the symptoms of the disease or condition. The number of cells injected into the patient in need of the treatment or prophylaxis may vary depending on inter alia, the antigen or antigens and size of the individual. This number may range for example between about 10³ and 10¹¹, and usually between about 10⁵ and 10⁷ cells (e.g., dendritic cells or T lymphocytes). Single or multiple administrations of the cells can be carried out with cell numbers and pattern being selected by the treating physician. The cells should be administered in a pharmaceutically acceptable carrier, which is non-toxic to the cells and the individual. Such carrier may be the growth medium in which the cells were grown, or any suitable buffering medium such as phosphate buffered saline. The cells may be administered alone or as an adjunct therapy in conjunction with other therapeutics known in the art for the treatment or prevention of unwanted immune responses for example but not limited to glucocorticoids, methotrexate, D-penicillamine, hydroxychloroquine, gold salts, sulfasalazine, TNFa or interleukin-1 inhibitors, and/or other forms of specific immunotherapy.

### 4. Preparation of Immunomodulating Compositions

The preparation of the immunomodulating compositions of the present invention uses routine methods known to persons skilled in the art. Typically, such formulations and vaccines are prepared as injectables, either as liquid solutions or suspensions; solid forms suitable for solution in, or suspension in, liquid prior to injection may also be prepared. The preparation may also be emulsified. The active immunogenic ingredients are often mixed with excipients that are pharmaceutically acceptable and compatible with the active ingredient. Suitable excipients are, for example, water, phosphate buffered saline, saline, dextrose, glycerol, ethanol, or the like and combinations thereof. In addition, if desired, the vaccine may contain minor amounts of auxiliary substances such as wetting or emulsifying agents, pH buffering agents, and/or adjuvants that enhance the effectiveness of the vaccine. Examples of adjuvants which may be effective include but are not limited to: surface active substances such as hexadecylamine, octadecylamine, octadecyl amino acid esters, lysolecithin, dimethyldioctadecylammonium bromide, N, N-dicoctadecyl-N′,N′bis(2-hydroxyethyl-propanediamine), methoxyhexadecylglycerol, and pluronic polyols; polyamines such as pyran, dextransulfate, poly IC carbopol; mineral gels such as aluminum phosphate, aluminum hydroxide or alum; peptides such as muramyl dipeptide and derivatives such as N-acetyl-muramyl-L-threonyl-D-isoglutamine (thur-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 1983A, referred to as MTP-PE), and RIBI, which contains three components extracted from bacteria, monophosphoryl lipid A, dimethylglycine, tuftsin; oil emulsions; trehalose dimycolate and cell wall skeleton (MPL+TDM+CWS) in a 2% squalene/Tween 80 emulsion; lymphokines; QuilA and immune stimulating complexes (ISCOMS). For example, the effectiveness of an adjuvant may be determined by measuring the amount of antibodies resulting from the administration of the vaccine, wherein those antibodies are directed against one or more antigens presented by the treated cells of the vaccine.

The active ingredients should be administered in a pharmaceutically acceptable carrier, which is non-toxic to the cells and the individual to be treated. Such carrier may be the growth medium in which the cells were grown. Compatible excipients include isotonic saline, with or without a physiologically compatible buffer like phosphate or Hepes and nutrients such as dextrose, physiologically compatible ions, or amino acids, and various culture media suitable for use with cell populations, particularly those devoid of other immunogenic components. Carrying reagents, such as albumin and blood plasma fractions and non-active thickening agents, may also be used. Non-active biological components, to the extent that they are present in the vaccine, are preferably derived from a syngeneic animal or human as that to be treated, and are even more preferably obtained previously from the subject. The injection site may be subcutaneous, intraperitoneal, intramuscular, intradermal, or intravenous.

If soluble actives are employed, the soluble active ingredients can be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric, maleic, and the like. Salts formed with the free carboxyl groups may also be derived from inorganic basis such as, for example, sodium, potassium, ammonium, calcium, or ferric hydroxides, and such organic basis as isopropylamine, trimethylamine, 2-ethylamino ethanol, histidine, procaine, and the like.

If desired, devices or pharmaceutical compositions or compositions containing the vaccine and suitable for sustained or intermittent release could be, in effect, implanted in the body or topically applied thereto for the relatively slow release of such materials into the body.

Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

The dosage to be administered may depend on the subject to be treated inclusive of the age, sex, weight and general health condition thereof. The dosage will also take into consideration the binding affinity of the inhibitor of IL-13 function, and in some embodiments the inhibitor of IL-4 function, to its target molecule, the immunogenicity of the immune stimulator, their bioavailability and their in vivo and pharmacokinetic properties. In this regard, precise amounts of the agent(s) for administration can also depend on the judgment of the practitioner. In determining the effective amount of the agent(s) to be administered in the treatment of a disease or condition, the physician or veterinarian may evaluate the progression of the disease or condition over time. In any event, those of skill in the art may readily determine suitable dosages of the agents of the invention without undue experimentation. Cell-containing compositions and vaccines are suitably administered to a patient in the range of between about 10⁴ and 10¹⁰, and more preferably between about 10⁶ and 10⁸ treated cells/administration. The dosage of the actives administered to a patient should be sufficient to effect a beneficial response in the patient over time such as a reduction in the symptoms associated with the cancer or tumor. For example usual patient dosages for systemic administration of inhibitors of IL-13 function, polypeptide antigens, or inhibitors of IL-4 function range from about 0.1-200 g/day, typically from about 1-160 g/day and more typically from about 10-70 g/day. Stated in terms of patient body weight, usual dosages range from about 1.5-3000 mg/kg/day, typically from about 15-2500 mg/kg/day, more typically from about 150-1000 mg/kg/day and even more typically from about 20-50 mg/kg/day.

Thus, the inhibitor of IL-13 function and the immune stimulator may be provided in effective amounts to stimulate or enhance the immune response to a target antigen. In some embodiments, an inhibitor of IL-4 function may also be provided in effective amounts to stimulate or enhance the immune response to a target antigen.

### 5. Methods for Modulating Immune Responses

The compositions of the invention may be used for stimulating an immune response to a target antigen in a subject that is immunologically naïve to the target antigen or that has previously raised an immune response to that antigen. Thus, the present invention also extends to methods for enhancing an immune response in a subject by administering to the subject the compositions or vaccines of the invention. Advantageously, the immune response is a cell-mediated immune response (e.g., a T-cell mediated response, which desirably includes CD8⁺ IFN-γ-producing T cells).

Also encapsulated by the present invention is a method for treatment and/or prophylaxis of a disease or condition, comprising administering to a patient in need of such treatment an effective amount of a inhibitor of IL-13 function, together with an effective amount of an immune stimulator, as broadly described above. In some embodiments, the method further may further comprise administering to the patient an inhibitor of IL-4 function, as broadly described above. In certain embodiments, the target antigen is associated with or responsible for a disease or condition which is suitably selected from cancers, infectious diseases and diseases characterised by immunodeficiency. Examples of cancer include but are not limited to ABL1 protooncogene, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, anal cancer, angiosarcoma, aplastic anemia, astrocytoma, ataxia telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, dermatofibrosarcoma protuberans, desmoplastic small round cell tumor, ductal carcinoma, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anemia, fibrosarcoma, gall bladder cancer, gastric cancer, gastrointestinal cancers, gastrointestinal carcinoid tumor, genitourinary cancers, germ cell tumors, gestational-trophoblastic disease, glioma, gynecological cancers, hematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's cell histiocytosis, laryngeal cancer, leiomyosarcoma, leukemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid tumor of kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small cell lung cancer (NSCLC), ocular cancers, esophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal tumors, pituitary cancer, polycythemia vera, prostate cancer, rare-cancers-and-associated-disorders, renal cell carcinoma, retinoblastoma, rhabdomyosarcoma, Rothmund thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's-macroglobulinemia, Wilms' tumor.

In other embodiments, the composition of the invention could also be used for generating large numbers of CD8⁺ or CD4⁺ CTL, for adoptive transfer to immunodeficient individuals who are unable to mount normal immune responses. For example, antigen-specific CD8⁺ CTL can be adoptively transferred for therapeutic purposes in individuals afflicted with HIV infection (Koup et al., 1991, J. Exp. Med. 174: 1593-1600; Carmichael et al., 1993, J. Exp. Med. 177: 249-256; and Johnson et al., 1992, J. Exp. Med. 175: 961-971), malaria (Hill et al., 1992, Nature 360: 434-439) and malignant tumours such as melanoma (Van der Brogen et al., 1991, Science 254: 1643-1647; and Young and Steinman 1990, J. Exp. Med., 171: 1315-1332).

In still other embodiments, the composition is suitable for treatment or prophylaxis of a viral, bacterial or parasitic infection. Viral infections contemplated by the present invention include, but are not restricted to, infections caused by HIV, Hepatitis, Influenza, Japanese encephalitis virus, Epstein-Barr virus and respiratory syncytial virus. Bacterial infections include, but are not restricted to, those caused by Neisseria species, Meningococcal species, Haemophilus species Salmonella species, Streptococcal species, Legionella species and Mycobacterium species. Parasitic infections encompassed by the invention include, but are not restricted to, those caused by Plasmodium species, Schistosoma species, Leishmania species, Trypanosoma species, Toxoplasma species and Giardia species.

The effectiveness of the immunisation may be assessed using any suitable technique. For example, CTL lysis assays may be employed using stimulated splenocytes or peripheral blood mononuclear cells (PBMC) on peptide coated or recombinant virus infected cells using ⁵¹Cr or Alamar Blue™ labeled target cells. Such assays can be performed using for example primate, mouse or human cells (Allen et al., 2000, J. Immunol. 164(9): 4968-4978 also Woodberry et al., infra). Alternatively, the efficacy of the immunisation may be monitored using one or more techniques including, but not limited to, HLA class I tetramer staining—of both fresh and stimulated PBMCs (see for example Allen et al., supra), proliferation assays (Allen et al., supra), ELISPOT assays and intracellular IFN-γ staining (Allen et al., supra), ELISA Assays—for linear B cell responses; and Western blots of cell sample expressing the synthetic polynucleotides.

In some embodiments, the composition comprises a nucleic acid construct from which an antigen that corresponds to the target antigen is expressible. Administration of such constructs to a mammal, especially a human, may include delivery via direct oral intake, systemic injection, or delivery to selected tissue(s) or cells. Delivery of the constructs to cells or tissues of the mammal may be facilitated by microprojectile bombardment, liposome mediated transfection (e.g., lipofectin or lipofectamine), electroporation, calcium phosphate or DEAE-dextran-mediated transfection, for example. A discussion of suitable delivery methods may be found in Chapter 9 of Ausubel et al., (1994-1998, supra).

The step of introducing the expression vector into the selected target cell or tissue will differ depending on the intended use and species, and can involve one or more of non-viral and viral vectors, cationic liposomes, retroviruses, and adenoviruses such as, for example, described in Mulligan, R. C., (1993). Such methods can include, for example:

A. Local application of the expression vector by injection (Wolff et al., 1990 Science 247 (4949 Pt 1): 1465-1468), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of the protein.

B. General systemic delivery by injection of DNA, (Calabretta et al., 1993), or RNA, alone or in combination with liposomes (Zhu et al., 1993), viral capsids or nanoparticles (Bertling et al., 1991) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by the expression vector, or of cells responsive to the protein. For example, in the case of a liposome containing antisense IL-13 polynucleotides and/or antisense IL-4 polynucleotides, the liposome may be targeted to skin cancer cells, e.g., squamous carcinoma cells, by the incorporation of immuno-interactive agents into the liposome coat which are specific the EGF receptor, which is expressed at higher levels in skin cancer.

C. Injection or implantation or delivery by any means, of cells that have been modified ex vivo by transfection (for example, in the presence of calcium phosphate: Chen et al., 1987, or of cationic lipids and polyamines: Rose et al., 1991), infection, injection, electroporation (Shigekawa et al., 1988) or any other way so as to increase the expression of the polynucleotide in those cells. The modification can be mediated by plasmid, bacteriophage, cosmid, viral (such as adenoviral or retroviral; Mulligan, 1993; Miller, 1992; Salmons et al., 1993) or other vectors, or other agents of modification such as liposomes (Zhu et al., 1993), viral capsids or nanoparticles (Bertling et al., 1991), or any other mediator of modification. The use of cells as a delivery vehicle for genes or gene products has been described by Barr et al., 1991 and by Dhawan et al., 1991. Treated cells can be delivered in combination with any nutrient, growth factor, matrix or other agent that will promote their survival in the treated subject.

In order that the invention may be readily understood and put into practical effect, particular preferred embodiments will now be described by way of the following non-limiting examples.

### EXAMPLES

### Example 1

### Materials and Methods

### Mice

Pathogen-free 6-8 weeks old female BALB/c, IL-4Rα^{-/-}, STAT6^{-/-}, IL-4^{-/-}, IL-13^{-/-}, and IL-4^{-/-}IL-13^{-/-} (H-2^{d} background) mice were obtained from the Animal Breeding Establishment, The John Curtin School of Medical Research, Australia. All animals were maintained and used in accordance with The John Curtin School of Medical Research animal ethics guidelines.

### Vaccines

AE FPV vaccines containing modified AE clade gag, pol, env, rev and tat genes were AE VV containing modified gag and pol genes were prepared as described previously (Ranasinghe et al., 2006, Vaccine 24: 5881-5895; Ranasinghe et al., 2007, J. Immunol. 178: 2370-2379; Coupar et al., 2006, Vaccine 24: 1378-1388) and as shown in the following table:

TABLE C

| | Insertion sites | | |
|---|---|---|---|
| Recombinant | F6,7-9 | TK-ORFX or TK | REV |
| FPV-117 (AE FPV) | AE gag/pol(m) | AE tat-rev | AE env(m) |
| VV-336 (AE VV) | | AE gag/pol(m) | |

TK = thymidine kinase,
ORFX = uncharacterised gene,
REV = reticuloendotheliosis provirus

### Immunisation of Mice and Preparation of T Cells

Mice (n=3-6) were primed with 1×10⁷ pfu rFPV and boosted with 1×10⁷ pfu rVv (expressing the AE clade HIV-1 antigens) 2 weeks later (under mild methoxyfluorane anesthesia) using i.n./i.n. (pure mucosal), i.n./i.m. (combined mucosal systemic), or i.m./i.m. (pure systemic) immunisation routes. In some experiments memory T-cell responses were recalled using i.r. (intra-rectal) AE VV challenge at 8 weeks following boost immunisation.

Before each immunisation the rFPV or rVV was diluted in sterile PBS and sonicated to obtain a homogeneous viral suspension. The recombinant viruses were mucosally administered in a final volume of 10-20 μL whereas i.m. immunisation was administered in a final volume of 100 μL.

Mice were sacrificed at different time intervals (2, 9 or 13 weeks) post-boost immunisation and systemic and/or mucosal T-cell responses were measured in splenocytes and/or genito-rectal node (iliac lymph node) cell suspensions prepared in complete RPMI as described below.

### IFN-Gamma ELIspot Assay

HIV-specific T-cell responses were measured by IFN-γ capture ELISpot assay as described previously (Ranasinghe et al., 2006, Vaccine 24: 5881-5895; Ranasinghe et aL, 2007, J. Immunol. 178: 2370-2379). Briefly, cells were stimulated for 16-20 hours in the presence of HIV-specific immuno-dominant H-2K^{d} binding AMQMLKETI, 9mer Gag peptide (the K^{d}Gag₁₉₇₋₂₀₅ tetramer) synthesised at the Bio-Molecular Resource Facility at The John Curtin School of Medical Research, Australia (Mata et al., 1998, J. Immunol. 161: 2985-2993). Con A (Sigma, USA) was used as the positive control and unstimulated cells as negative controls. The spot forming units were counted using an ELISpot Bio Reader-4000 (BIOSYS, GmbH, Germany).

Results are expressed as 1×10⁶ T cells and represent the average of the duplicate or triplicate value. Unstimulated cell counts were subtracted from stimulated counts before plotting the data.

### Tetramer Staining and Cell Sorting

The K^{d}Gag₁₉₇₋₂₀₅ tetramer staining was performed as described previously (Ranasinghe et al., 2006, Vaccine 24: 5881-5895). Briefly, 5×10⁶ cells were stained (unstimulated with any peptide) with anti-CD8 FITC antibody (BD Pharmingen, San Diego, Calif., USA) and allophycocyanin-conjugated K^{d}Gag₁₉₇₋₂₀₅ tetramer in FACS buffer not containing azide, for 40 minutes at room temperature.

After two washes, K^{d}Gag₁₉₇₋₂₀₅-specific single cells were sorted into 96-well plates for single-cell multiplex nested PCR analysis and snap frozen and kept at −80° C. until use, or where necessary were sorted and cultured for 3-4 days before intracellular cytokine staining.

### Single-Cell cDNA Synthesis and Single-Cell PCR

To synthesise the cDNA, 5 μL cDNA buffer (Sensiscript RT kit, QIAGEN), containing 0.5 mM dNTP (QIAGEN), 125 ng oligo dT (Promega, USA), 2.5 U RNAsin (Promega), 0.5 nM spermidine (Sigma), 0.1% Triton-X 100 (Sigma), 100 μg/mL tRNA, and 0.125 μL Sensiscript Reverse Transcriptase (Sensiscript RT kit, QIAGEN), was added to each cell and each well centrifuged at 2500 rpm for 3 minutes and incubated at 37° C. for 110 minutes (Ranasinghe et al., 2007, J. Immunol. 178: 2370-2379; Turner et al., 2003, Immunity 18: 549-559).

Nested PCR was performed as described previously (Ranasinghe et al., 2007, J. Immunol. 178: 2370-2379) using HotStar Taq Master mix (QIAGEN) with 5 pmol of forward and reverse cytokine and granzyme B primers (as indicated in Ranasinghe et al., 2007, J. Immunol. 178: 2370-2379) plus CCL5 PCR1 primers 5′-3′ (FW1: ATGAAGATCTCTGCAGCTGCC and REV1: CTAGCTCATCTCCAAATAGTT) and PCR2 primers 5′-3′ (FW2: TCACCATCATCCTCACTGCAG and REV2: TCGAGTGACAAACACGACTGC). Most of these primers were designed using Primer Bank (Wang et al., 2003, Nucleic Acids Res. 31: 154).

Results are represented as a percentage of tetramer reactive cells (K^{d}Gag₁₉₇₋₂₀₅-specific CTL) expressing the cytokine, granzyme B or CCL5.

### Tetramer Dissocation Assay

The dissociation assay was performed as described elsewhere (La Gruta et al., 2004, J. Immunol. 172: 5553-5560; Ranasinghe et al., 2007, J. Immunol. 178: 2370-2379). Briefly, 2×10⁶ cells from each sample were stained with FITC-CD8α and allophycocyanin-labelled K^{d}Gag₁₉₇₋₂₀₅ as described above.

50 μg/μL of H-2K^{d} competitive antibody (BD Pharmingen) was added to each well to prevent tetramer re-binding and plates were incubated at 37° C., 5% CO₂.

At each time point, aliquots were transferred to cold FACS buffer, then washed and resuspended in 100 μL of of FACS buffer containing 0.5% paraformaldehyde. These samples were analysed on a FACSCalibur flow cytometer (Becton-Dickinson) using Cell Quest Pro analysis software.

### Cell Surface Cytokine Staining and Intracellular Cytokine Stainins (ICS)

For all cytokines, except IL-2 and IFN-γ, in total, 2×10⁶ lymphocytes were stimulated for 16 hours in the presence of immuno-dominant H-2K^{d} binding AMQMLKETI 9mer Gag peptide and then for a further 5-6 hours in the presence of brefeldin A, as described previously (Ranasinghe et al., 2006, Vaccine 24: 5881-5895).

Owing to the different expression kinetics of cytokines IL-2 and IFN-γ (Ranasinghe et al., 2007, J. Immunol. 178: 2370-2379), when IL-2 production was assessed, cells were only stimulated for 4 hours,and when IFN-γ production was assessed, these cells were first cultured in complete RPMI in the presence of IL-2 for 3-4 days, then the cells were mixed with naïve splenocytes, and then re-stimulated with 9mer Gag peptide, after which the cells were stimulated initially for 1 hour and then for a further 5 hours in the presence of brefeldin A.

Following stimulation, cells were surface stained with anti-CD8 allophycocyanin or FITC and/or CD62L FITC (BD Pharmingen). These cells were fixed and permeabilised before intracellular staining with anti-mouse IFN-γ FITC, IL-2 FITC or IL-4 allophycocyanin (BD Pharmingen).

One hundred thousand gated events from each sample were acquired on a four-colour FACSCalibur flow cytometer, and results were analysed using Cell Quest Pro software. Unstimulated cell counts were used as the background controls.

### Cytoline Antibody Arrays

In total 2×10⁶ splenocytes were cultured in complete RPMI without IL-2 for 16-20 hours in the presence of the Gag₁₉₇₋₂₀₅ peptide. Supernatants were collected and cytokine antibody arrays were performed according to the manufacturer's instructions (Chemicon International or Ray Biotech, USA).

Cytokine expression was detected using chemoluminescence substrate. Expression signal intensities of each cytokine were calculated as a percentage absorbance, normalised against the positive controls on the membrane using Multi Gauge V3.0 software density linear calibration analysis (A−B/mm²; where A=average absorbance of the cytokine, B=average background absorbance, mm²=average area).

Then the final percentage absorbance for each cytokine was calculated by subtracting un-immunised percentage absorbance from immunised percentage absorbance.

### Statistics and Analysis of Data

For all assays (where appropriate)+SD was calculated and p-values were determined using a two-tailed, two-sample equal variance or unequal variance Student's t-test. Except where stated, experiments were repeated three or more times.

### Results

### Musocsal Immunisation Elicits CD8⁺ T Cells of Higher Avidity than Immunisation by Systemic Route

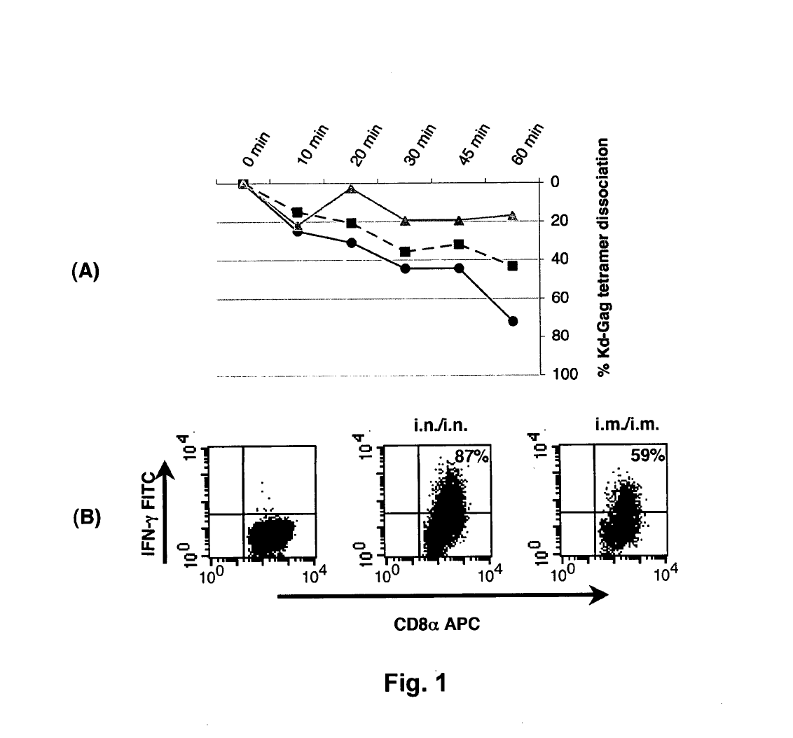

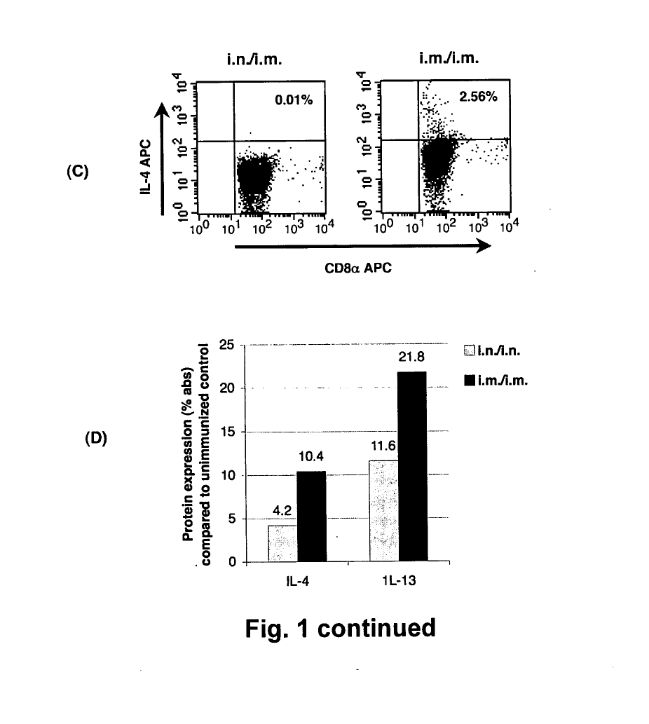

It was found in the tetramer dissociation assay that K^{d}Gag₁₉₇₋₂₀₅-specific CTL following i.m/i.m. immunisation (pure systemic) elicited T cells with the highest tetramer dissociation (e.g., lowest avidity), i.n./i.m. immunisation (combined systemic mucosal) elicited intermediary levels of tetramer dissociation and i.n./i.n. immunisation (pure mucosal) regime demonstrated the lowest tetramer dissociation (e.g., highest avidity). This data is shown in FIG. 1A and clearly demonstrates that mucosal immunisation generates HIV-specific CTL of higher avidity compared with systemic immunisation. This data was previously reported in Ranasinghe et al., 2007, J. Immunol. 178: 2370-2379.

To assess whether there is a significant difference in the percentage of K^{d}Gag₁₉₇₋₂₀₅-specific CTL that expressed IFNγ after i.n./i.n. or i.m./i.m. immunisations, K^{d}Gag₁₉₇₋₂₀₅-specific CTL were FACS sorted, following tetramer staining and were re-stimulated with Gag₁₉₇₋₂₀₅-specific peptide in vitro as described in the Materials and Methods section above. The data from the results obtained revealed that the capacity to produce IFN-γ by CTL was vaccine route dependent, as ˜87% of K^{d}Gag₁₉₇₋₂₀₅-specific CTL from i.n./i.n. immunised group were IFN-γ⁺ while only ˜59% of K^{d}Gag₁₉₇₋₂₀₅-specific CTL from i.m./i.m. immunised group were able to express IFN-γ⁺ (as shown in FIG. 1B).

### HIV-Specific CTL from Systemically Immunised Mice Produce Higher Levels of IL-4 and IL-13

Using single-cell multiplex nested PCR analysis, it has been previously demonstrated that K^{d}Gag₁₉₇₋₂₀₅-specific CTL obtained from the i.m./i.m. immunised group showed much higher numbers of Th2 cytokine IL-4 mRNA producing cells (>20%) compared with mucosally immunised group (˜4%) (Ranasinghe et al., 2007, J. Immunol. 178: 2370-2379). It was also shown that the number of CTL that expressed IL-4 mRNA was also greater in memory population than effector cell pool.

Hence, in these studies, 8 weeks following prime boost immunisation, intrarectal (i.r.) AE VV challenge was performed to recall memory and to overcome the barrier of preexisting VV immunity in the systemic compartment. This model has been extensively studied (Belyakov et al., 1999, Proc. Natl. Acad. Sci. USA 96: 4512-4517), and accepted as a way of overcoming vector-induced immunity. This enabled the inventors to further substantiate that greater percentage of memory CD8⁺CTL obtained from i.m./i.m. immunised mice expressed IL-4 compared with i.n./i.m. delivery (2.56% versus i.n./i.m. 0.01%) as shown in FIG. 1C.

Moreover, when similar numbers of splenocytes from i.n./i.n. and i.m./i.m. immunised mice were cultured overnight in the presence of Gag₁₉₇₋₂₀₅ peptide and supernatants were collected and analysed by cytokine antibody arrays, the data clearly indicated that CD8⁺T cells obtained from systemically immunised cells expressed two-fold higher IL-4 and IL-13 protein compared with mucosally immunised cells as shown in FIG. 1D.

### HIV-Specific CTL in IR-4RALPHA^{-/-} and BALB/c Mice

IL-4Rα is a common receptor for 1L-4 and IL-13. Therefore, to further evaluate the role of IL-4 and IL-13 expression and their influence on CTL avidity, IL-4Rα^{-/-} and wild type BALB/c mice (H-2^{d} background) were prime boosted i.n./i.m. with AE FPV/AE VV. The combined i.n./i.m. immunisation regime was particularly chosen for avidity studies because, previously it has been shown that i.n./i.m. immunisation regime generated robust systemic and mucosal immunity to vaccine antigens (Ranasinghe et al., 2006, Vaccine 24: 5881-5895) and also elicited intermediary levels of tetramer dissociation compared with i.n./i.n. or i.m./i.m. immunisation regimes (Ranasinghe et al., 2007, J. Immunol. 178: 2370-2379). Hence, this regime allowed monitoring of both increases and/or decreases in tetramer loss. By comparison, the T cells from i.n./i.n. immunised mice had previously showed minimal tetramer dissociation, and therefore any further decrease would have been difficult to assess.

Two weeks following immunisation, cytokine profiles of antigen-specific CD8⁺ CTL were evaluated using a range of techniques. Even though similar numbers of tetramer reactive CTL were detected between IL-4Rα^{-/-} and wild type BALB/c mice (see FIG. 2A), IL-4Rα^{-/-} mice showed significantly reduced number of cells expressing IFN-γ as assessed by ELISpot (IL-4Rα^{-/-} p=0.018) and IFN-γ intracellular cytokine staining (IL-4Rα^{-/-} p=0.016) compared with wild type BALB/c control (see FIGS. 2B and 2C respectively).

Furthermore, upon K^{d}Gag₁₉₇₋₂₀₅ peptide stimulation, expression of IL-2 protein was significantly higher in IL-4Rα^{-/-} CD8⁺T cells (˜7.69%) compared with BALB/c control (˜0.65%) as shown in FIG. 2D. In contrast reduced IFN-γ expression was observed compared to the controls.

Single-cell multiplex nested PCR analysis of un-stimulated K^{d}Gag₁₉₇₋₂₀₅-specific CTL also confirmed these findings with over 20% of cells expressing IL-2 compared with wild type BALB/c (0%) as shown in FIG. 3A. Significantly lower numbers of IFN-γ⁺ HIV-specific CTL were observed in IL-4Rα^{-/-} mice.

There were no differences in IL-4 expressing numbers in IL-4Rα^{-/-} mice compared with wild type BALB/c control mice, yet increased IL-13 expressing K^{d}Gag₁₉₇₋₂₀₅-specific CTL numbers were observed (see FIG. 3A). The data here indicated that the absence of the receptor did not influence the secretion of IL-4 and IL-13 cytokines. Furthermore, IL-4Rα^{-/-} K^{d}Gag₁₉₇₋₂₀₅-specific CTL tetramer loss was also not significantly different compared with wild type BALB/c CTL at 60 minutes end time point (as shown in FIG. 3B).

### HIV-Specific ‘Effector’ of Higher Avidity are Found in IL-13^{-/-} KO Mice

To further evaluate the influence of these Th2 cytokines on CTL avidity similar experiments were performed with IL-13^{-/-}, IL-4^{-/-}, IL-13^{-/-}IL-4^{-/-}, and STAT6^{-/-} KO mice (H-2^{d} background) following i.n./i.m. poxvirus prime boost immunisation. Tetramer analysis revealed that the number of K^{d}Gag₁₉₇₋₂₀₅-specific effector CTL between the KO and BALB/c control did not differ markedly (as shown in FIG. 4A), although lower numbers were detected in mice compared with the other two KO groups (IL-4^{-/-} and STAT6^{-/-} p=0.016; IL-4^{-/-} and IL-13^{-/-} p=0.023; IL-4^{-/-} and BALB/c p=0.240). In contrast, KO mice showed significantly different IFN-γ ELISpot counts compared with wild type BALB/c control. The STAT6^{-/-} mice showed the highest IFN-γ counts and IL-4^{-/-} mice the lowest (as shown in FIG. 4B).

When tetramer loss of the KO K^{d}Gag₁₉₇₋₂₀₅-specific effector CTL was evaluated at 60 minutes compared with wild type BALB/c, IL-13^{-/-} CTL showed the slowest tetramer dissociation (p=0.043), followed by IL-4^{-/-} (p=0.045) and STAT6^{-/-} (p=0.115) (as shown in FIG. 4C).

Interestingly, single-cell multiplex nested PCR showed no IL-4 or IL-13 expression in IL-13^{-/-} or IL4^{-/-} HIV-specific effector CTL, respectively. Moreover, between the two groups, Th1 cytokine/granzyme B profiles (as shown in FIG. 4E) as well as effector CTL avidity profiles were also remarkably similar.

Surprisingly, IL-4^{-/-}IL-13^{-/-} double KO T cells showed reduced K^{d}Gag₁₉₇₋₂₀₅-specific tetramer staining and IFN-γ responses compared with IL-13^{-/-} T cells (see FIG. 4D). Single-cell multiplex PCR analysis of double cytokine KO HIV-specific CTL also revealed that the cytokine profiles they produced were relatively different to single cytokine KO mice (see FIG. 4E) or wild type BALB/c mice.

### HIV-Specific ‘Memory’ CTL of Higher Avidity are Observed in IL-13^{-/-} and STAT6^{-/-} Mice

Studies were also performed to evaluate the ‘quality’ of the memory CTL population following a mucosal i.r. AE VV memory recall at 8 weeks post-booster immunisation (similar to previous studies i.r. re-exposure of AE VV was used to overcome the barrier of preexisting VV immunity in the systemic compartment (Belyakov et al., 1999, Proc. Natl. Acad. Sci. USA 96: 4512-4517)). Although significant differences in T-cell responses were observed by K^{d}Gag₁₉₇₋₂₀₅-specific tetramer staining (see FIG. 5A) and IFN-γ ELISpot (see FIG. 5B) between STAT6^{-/-} and IL-4^{-/-} compared with wild type control, similar T-cell responses were detected between IL-13^{-/-} and control BALB/c.

In contrast, upon Gag peptide stimulation, a higher percentage of K^{d}Gag₁₉₇₋₂₀₅-specific CD62L⁺ cells was detected in IL-13^{-/-} mice compared with all groups tested (see FIG. 5C).

Furthermore, when functional avidity of these CTL was measured, slowest tetramer loss was observed in IL-13^{-/-} (p=0.0011), then STAT6^{-/-} (p=0.0035) and IL-4^{-/-} (p=0.1230) compared with wild type control at 60 minutes end time point (see FIG. 6). It is believed that tetramer dissociation assays provide a more functional measure of CTL avidity compared dose response IFN-γ ELISpot, independent of IFN-γ measurement (La Gruta et al., 2004, J. Immunol. 172: 5553-5560).

### Higher Numbers of HIV-Specific Memory CTL Expressing IFN-Gamma are Found in IL-13^{-/-} and STAT6^{-/-} Mice

Single-cell multiplex nested PCR of K^{d}Gag₁₉₇₋₂₀₅-specific CTL (unstimulated CTL) enables the evaluation of ‘crucial micro level changes in cells, in a naïve state’ (cells not being restimulated in vitro), which are otherwise undetectable or unfeasible using other techniques (ELISpot, intracellular cytokine staining (ICS)) due to small sample size.

To uncover the differences observed in CTL avidity, single-cell cytokine profiling was performed on systemic (splenocytes) and mucosal (genito-rectal nodes) memory K^{d}Gag₁₉₇₋₂₀₅-specific CTL from KO and control mice (n=96 cells). The method was as described in the Materials and Methods section and in Ranasinghe et al., 2007, J. Immunol. 178: 2370-2379. Surprisingly, the results here showed low numbers of BALB/c K^{d}Gag₁₉₇₋₂₀₅-specific memory CTL expressed IFN-γ (<2%). In striking contrast, a much greater proportion of IL-13^{-/-} K^{d}Gag₁₉₇₋₂₀₅-specific systemic and mucosal memory CTL expressed IFN-γ (58% or 73%) as shown in the following table:

TABLE D
SINGLE CELL CYTOKINE PROFILES OF SYSTEMIC AND MUCOSAL K^{D}GAG₁₉₇₋₂₀₅-SPECIFIC MEMORY CTL

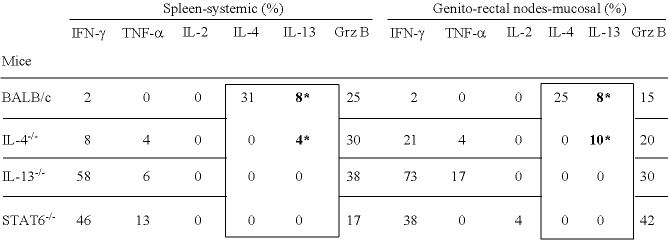

The data in the above table represent the % of K^{d}Gag₁₉₇₋₂₀₅-specific splenocytes and genito-rectal nodes producing IFN-γ, TNF-α, IL-2, IL-4, IL-13 and granzyme B. The box highlights differences in IL-4 and IL-13 expression in different groups. (*)highlights the IL-13 expression in HIV-specific CTL. Data are representative of two experiments.

This data suggested that in a memory CTL population the number of cells expressing IFN-γ was inversely related to the expression of IL-13 by CTL (e.g., IL-13^{-/-}>STAT6^{-/-}>IL-4^{-/-}>control). Granzyme B expression was also highest in IL-13^{-/-} CTL (38%). Furthermore, over 25% of the wild type BALB/c K^{d}Gag₁₉₇₋₂₀₅-specific memory CTL also expressed IL-4, but no IL-4 was detected in any other group as shown in Table D above. IL-13 was found both in wild type BALB/c and IL-4^{-/-} HIV-specific CTL. Low numbers of cells expressing TNF-α were also detected in splenocytes and genito-rectal nodes from KO animals but none were found in the wild type BALB/c mice.

Unlike wild type BALB/c memory CTL, there were also observed differences in cytokine expression between systemic and mucosal K^{d}Gag₁₉₇₋₂₀₅-specific memory CTL obtained from KO mice (as shown in Table D above). Interestingly, over 4% STAT6^{-/-} K^{d}Gag₁₉₇₋₂₀₅-specific genito-rectal memory CTL also showed IL-2 expression, whereas IL-2 expressing cells were not detected in BALB/c mice or cytokine KO CTL (as shown in Table D above).

In summary, higher IFN-γ expression and no IL-4 or IL-13 expression were detected in IL-13^{-/-} and STAT6^{-/-} K^{d}Gag₁₉₇₋₂₀₅-specific memory CTL, these cells were also higher in avidity (slower tetramer loss) (see Table D and FIG. 6). Expression of IL-4 and IL-13 was greatly enhanced in wild type BALB/c CTL, which also showed significantly lower IFN-γ expression, and the highest tetramer loss compared with other groups tested (see Table D and FIG. 6).

Intriguingly, IL-13 was also expressed in IL-4^{-/-} memory CTL, which were lower in avidity. Thus, these data clearly indicate that Th2 cytokine IL-13 expression plays a more pivotal role in down-regulating the functional avidity of CTL than IL-4.

Recent in vitro studies have further substantiated these findings showing that when IL-13 was reconstituted in IL-13^{-/-} T cells, their ability to bind to tetramers or produce IFN-γ upon K^{d}Gag₁₉₇₋₂₀₅ peptide stimulation was greatly reduced compared with control treatment.

### Both CTL Avidity and CCL5 Expression are Inversely Related to the Expression of IL-4/IL-13

It has been observed recently in micro-array studies that following prime boost immunisation memory CD8⁺ cells maintain high levels of untranslated chemokine CCL5 (RANTES) mRNA that is activated upon peptide stimulation, and similar observations have been reported by Walzer et al., 2003, J. Immunol. 170: 1615-1619.

It has also been demonstrated that IL-4 treatment of murine CD8⁺ T cells can inhibit CCL5 (Marcais et al., 2006, J. Immunol. 177: 4451-4457), while CCL5 can also inhibit IL-4 expression in a CCLI-dependent manner (Chensue et al., 1999, J. Immunol. 163: 165-173).

Therefore, in K^{d}Gag₁₉₇₋₂₀₅-specific memory CTL CCL5, IL-4 and IL-13 levels were also evaluated by single-cell multiplex nested PCT. IL-13^{-/-} K^{d}Gag₁₉₇₋₂₀₅-specific memory CTL showed the highest number of CTL expressing CCL5 (90%) compared with IL-4^{-/-} (75%) and lowest was detected in BALB/c mice (30%) (see FIG. 7).

Furthermore, high-avidity CTL obtained from IL-13^{-/-} KO mice elicited high numbers of CCL5 and IFN-γ expressing cells, while no IL-4 or IL-13 was detected in these CTL (see FIG. 7).

In contrast, wild type BALB/c HIV-specific CTL showed reduced IFN-γ (<2%), CCL5 (31%) expressing CTL but showed high numbers of CTL expressing IL-4 (30%) and IL-13 (4%) (see FIG. 7), which were lower in avidity.

These data clearly indicate that IL-4 and IL-13 cytokines can modulate CCL5 expression.

### Discussion

Correlates of protective immunity for many infections are poorly defined but it becoming increasingly apparent that the avidity of T cells is an important parameter for immunological defence. The capacity to resist infection is in part related to the avidity of T cells. The results from the experimental work discussed above show that IL-13 directly influences the avidity of T cells that are induced by vaccine antigens. This was shown using vaccinated IL-13 gene knock-out mice in which T cells showed high levels of avidity and more importantly could protect against a challenge virus.

It is believed that a recombinant vaccine co-expressing an antigen and an IL-13 inhibitor (such as IL-13Rα2Δ10) will be able to elicit T cells with higher avidity to antigens and a capacity to protect against a pathogenic challenge. Thus the co-expression of an antigen and a soluble IL-13 antagonist in a recombinant vaccine will be able to dramatically enhance T cell avidity elicited to vaccine antigens and afford a higher level of protection against challenge.

It is believed that the vector of the vaccine will enter cells of the host, some of which will be antigen-presenting cells of the immune system, and expresses both the antigen and the IL-13 antagonist. The antigen is processed by the cell, so as to stimulate an immune response specific for the antigen, while the IL-13 antagonist leaves the cell and binds to host IL-13 that is produced during the immune response. The expression of both the antigen and IL-13 antagonist occurs in the local milieu of the immune response. It is believed that production of both the antigen and IL-13 antagonist in the local milieu of the immune response may be an essential requirement for the desired immune response (production of high avidity CD8+ T cells). It is believed that delivery of the antigen and IL-13 antagonist separately fails to induce appropriate responses. In the recombinant vaccine of the present invention, the IL-13 antagonist binds and therefore detracts host IL-13 from its negative effect on the immune response resulting in heightened T cells responses with increased avidity towards the antigen. The T cells elicited under this regime also have markedly broadened cytokine profile responses different from that induced without the IL-13 antagonist.

### Example 2

### Materials and Methods

### Genes and Plasmids

A spleen was removed from a female C57BL/6 mouse and immediately immersed in RNAlater stabilisation reagent (QIAGEN) and stored at −20° C. Total RNA was isolated from 10 mg of stabilised spleen tissue using the RNeasy Protect Mini Kit (QIAGEN) as recommended by the manufacturer.

Mouse IL-13Rα2 cDNAs were amplified from the total RNA using gene specific primers AGATCTGAAATGGCTTTTGTGCATATCAGATGCTTGTG and GAGCTCTTAACAGAGGGTATCTTCATAAGC and the One-Step RT-PCR Kit (QIAGEN) as recommended by the manufacturer.

The PCR products were purified using the Mini-Elute Gel Purification Kit (QIAGEN) and directly ligated into the U-tailed vector pDrive and used to transform QIAGEN EZ competent cells contained in the PCR Cloning-Plus Kit (QIAGEN).

Two different length PCR products were isolated, a 1167 bp product encoding the full-length membrane bound IL-13Rα2 and a 1051 bp splice variant encoding the secreted IL-13 receptor (sIL-13Rα2; IL-13Rα2Δ10) which lacks exon 10 and thus the trans-membrane domain sequences (Tabata et al., 2006, J. Immunol. 177: 7905-7812). The DNA sequences encoding the IL-13α2 cDNAs isolated here were identical to GenBank entries NM008356 and EF219410 (secreted splice variant).

The cloned PCR product containing the IL-13Rα2 sequence was digested with BglII and SacI and the fragment gel-purified and ligated between the BamHI and Sad sites of vaccinia virus (VACV) vector pTK7.5A (Coupar et al., 1988, Gene 68: 1-10) immediately downstream of the P7.5 early/late promoter. The IL-13Rα2 cDNA was also isolated on BglII and PstI (pDrive MCS) DNA fragments and ligated between the BamHI and Pstl sites of the fowlpox virus (FPV) vector pAF09 (Heine and Boyle, 1993, Arch. Viral. 131: 277-292) downstream of the FPV early/late promoter and in-frame with the upstream ATG.

### Recombinant Viruses

Recombinant poxviruses co-expressing the HIV gag/pol antigen and mouse IL-13Rα2 were constructed using parent viruses FPV-086 and VV-336 (Coupar et al., 2006, Vaccine 24: 1378-1388). Recombinant FPV was constructed by infecting chicken embryo skin (CES) cell cultures with FPV-086 (MOI 0.05) followed by transfection with pAF09-IL-13Rα2 using Lipofectamine 2000 transfection reagent (Invitrogen).

Recombinant viruses were selected by passage of viruses on CES cells in the minimal essential media (MEM) containing 5% (v/v) foetal bovine sera (FBS) and MX-HAT (2.5 μg/mL mycophenolic acid), 250 μg/mL xanthine, 100 μg/mL hypoxanthine, 0.4 μg/mL aminopterine and 30 μg/mL thymidine) to select for viruses expressing the gpt (xanthine guanine phosphoribosyltransferase) gene.

Plaques containing recombinant viruses were identified using an agar overlay (1% agar in MEM) containing X-gal (200 μg/mL) to detect co-expression of the lacZ gene. Blue staining plaques were picked and further plaque purifications (3 or 4 in total) conducted using selective media. Recombinant viruses were confirmed by PCR for the presence of the IL-13Rα2 gene and absence of wild-type virus insert site sequences.

Recombinant VV was similarly constructed by infecting H143B TK-cells with VV-336 (MOI 0.05) and transfection with pTK7.5A-IL-13Rα2Δ10. Recombinant viruses were selected using MEM containing HAT supplement (100 μg/mL hypoxanthine, 0.4 μg/mL aminopterine and 30 μg/mL thymidine) to select for viruses expressing the HSV TK gene contained in the vector. Plaques growing in selective media were plaque purified and confirmed for the IL-13Rα2 gene and absence of virus wild-type insertion site by PCR.

### Immuno-Blotting

Expression of IL-13Rα2 by the recombinant viruses was confirmed by immuno-blotting of infected cells and clarified culture media.

Confluent monolayers of either H143B TK- or CES in 24 well plates were infected at MOI of 1 PFU/cell of recombinant vaccinia or fowlpox viruses, respectively.

Infected cells were incubated in 0.5 mL MEM, 5% FBS at 37° C. At 72 hours post-infection with vaccinia virus the infected cells were scraped from the plates, cell debris separated by centrifugation and media recovered and stored at −20° C. The cell pellet, approximately 5×10⁵ cells, was resuspended in 100 μL SDS-PAGE cell extraction buffer (2% (w/v) SDS, 60 mM Tris-HCl pH 6.8, 0.75 M β-mercaptoethanol, 0.01% (w/v) bromophenol blue) containing 1× Complete protease inhibitor EDTA-free (Roche) and pushed through a 26-gauge needle to reduce viscosity. Fowlpox virus infected cells were similarly processed at 120 hours post infection.

Aliquots of 10 gL SDS lysed cells or 20 μL cell culture media were mixed with an equal volume of TrueSep sample buffer (2×) (NuSep, BG-165) containing β-mercaptoethanol, heat denatured and applied to 4-20% polyacrylamide long-life gels (NuSep, NH21-420) and proteins separated by electrophoresis using a Tris-HEPES-SDS buffer (NuSep, BG-163) as recommended by the manufacturer.

Proteins were transferred to 0.45 μm pore Immobilon-P membraes (Millipore) using standard transfer tank blotting procedures in 25 mM Tris base, 192 mM glycine and 10% (v/v) methanol at 8 V/cm inter-electrode distance as recommended by the membrane manufacturer.

Recombinant mouse IL-13Rα2 bound to Immobilon-P membrane was detected by the rapid immune-detection procedure as recommended by the manufacturer using goat anti-mouse IL-13Rα2 polyclonal sera (R&D Systems, AF539) at 0.2 μg/mL in PBS containing 0.05% (v/v) Tween-20, 0.5% (w/v) skim milk powder for 60 minutes. Secondary rabbit anti-goat IgG biotin conjugate (Sigma, B7014) diluted 1:1000 was applied to the membrane followed by streptavidin-horseradish peroxidise conjugate (Amersham, RPN 1231 V) diluted 1:1000, washing with PBS/Tween-20 after each incubation.

The filters were developed using Western Lightning™ Chemiluminescence Reagent Plus (PerkinElmer, NEL103) and images captured using Fujifilm LAS 1000 Luminescence Image Analyser.

### Further Methods and Results

### Avidity and Protective Capacity of CD8⁺ T Cells in Absence of IL-13

Normal (Balb/c) and IL-13^{-/-} mice were immunised against HIV gag/pol antigens using a prime-boost vaccination regime of recombinant FPV and VV expressing HIV AE gag/pol.

Although the number of K^{d}Gag₁₉₇₋₂₀₅-specific CD8 T cells was similar in normal and IL-13^{-/-} mice, the IL-13^{-/-} mice were more resistant to influenza-K^{d}Gag₁₉₇₋₂₀₅ challenge than the normal (control) mice.

As shown in Example 1, CD8⁺ K^{d}Gag₁₉₇₋₂₀₅-specific T cells from vaccinated IL-13^{-/-} mice showed a lower dissociation rate (i.e. higher avidity) compared with CD8⁺ T cells from normal mice (FIG. 8A).

Next, spleen cells from prime-boost immunised IL-13^{-/-} or control mice were transferred to naïve animals and the naïve animals were challenged with influenza virus encoding K^{d}Gag₁₉₇₋₂₀₅. Although the transfer of T cells from immunised control mice gave a marginal level of protection from influenza-K^{d}Gag₁₉₇₋₂₀₅ challenge, animals receiving IL-13^{-/-} T cells were completed protected (see FIG. 8B—weight loss is accepted as the standard observation of influenza challenge).

The immune responses after influenza-KdGag197-205 challenge were measured using IFN-γ ELISpot and intracellular cytokine staining (ICS). Mice receiving IL-13−/− spleen cells responded substantially better than animals receiving cells from vaccinated normal mice. The enhanced response of IL-13−/− spleen cells was also reflected in the number of CD8+ T cells expressing both IFN-γ and TNF-α. The superior responses with IL-13−/− CD8 T cells were observed whether the donors were obtained from i.m. or i.n. immunised animals (see FIG. 8C).

### Soluble IL-13 Decoy

IL-13 is regarded as an immunoregulatory cytokine secreted predominantly by the activated T-helper type 2 (Th2) cell, although CD8 T cells can be induced to express and respond to the cytokine.

The various functions of IL-13 are mediated by a complex receptor system. IL-13Rα1 (CD213α1) binds IL-13 with low-affinity, however when paired with IL-4Rα (CD124) it binds with high affinity forming the functional IL-13 receptor (also known as the IL-4 Type II receptor) that results in cell signaling via the STAT6 pathway. A second receptor, IL-13α2, has been identified which binds IL-13 with high affinity. The membrane associated IL-13Rα2 has a short cytoplasmic domain that does not contain known signaling motifs and it has been postulated that it may act as a decoy receptor. Soluble IL-13Rα2 has been identified in vivo that can sequester IL-13 preventing it binding to its receptor. Soluble IL-13Rα2 is thought to result from either cleavage of the extracellular IL-13 binding domain or, at least for the mouse, results from alternate mRNA splicing producing both cell membrane bound IL-13Rα2 and a secreted IL-13Rα2 lacking the trans-membrane motif.

Based on the above and the results already observed, the inventors reasoned that a recombinant vaccine vector expressing the decoy soluble IL-13Rα2 would bind and inhibit IL-13 activity resulting in the induction of high avidity CD8 T cells.

Recombinant FPV and VV vectors encoding HIV gag/pol and IL-13Rα2 were constructed and shown to express gag/pol antigens and IL-13Rα2 during in vitro infection of tissue culture cells (see FIG. 9). Administration of FPV gag/pol IL-13Rα2 and VV gag/pol IL-13Rα2 in a prime-boost vaccination regime was compared with FPV gag/pol and VV gag/pol. Balb/c mice elicited higher numbers of K^{d}Gag₁₉₇₋₂₀₅ specific CD8 T cells when given FPV gag/pol IL-13Rα2 and FPV gag/pol compared to those given FPV gag/pol and VV gag/pol. Indeed the responses obtained were higher than those induced in IL-13^{-/-} mice given FPV gag/pol and VV gag/pol (FIGS. 10A-D). The elevated responses elicited were shown by tetramer staining (FIG. 10A), intracellular IFN-g expression (FIG. 10B), IL-2 expression by T cells (FIG. 10C) and TNF (FIG. 10D) as a monitor of polyfunctional T cells. More importantly, when the inventors evaluated the influence of IL-13Rα2 expression on the avidity of the K^{d}Gag₁₉₇₋₂₀₅ specific CD8 T cells elicited, they found the slowest tetramer dissociation (highest avidity) in mice immunised with FPV gag/pol IL-13Rα2 and VV gag/pol IL-13Rα2 (FIG. 11).

Prime boost immunisation using various vaccine vectors has been extensively explored as a vaccine strategy to induce good humoral and cell-mediated immune responses. The generally accepted explanation for the success of this strategy over repeated viral vector delivery is the circumvention of anti-vector immunity that can hinder the immunity elicited with a subsequent viral boost (Ramshaw, I. A., and Ramsay, A. J., 2000 Immunol Today 21(4): 163-5). To examine whether inhibition of IL-13 by IL-13Rα2 was required in the prime or boost part of the vaccination regime we immunised mice with FPV gag/pol IL-13Rα2 and boosted with VV gag/pol or primed with FPV gag/pol and boosted with VV gag/pol IL-13Rα2. The avidity of the resulting CD8 T cell population was compared with CD8 T cells induced when both vectors expressed IL-13Rα2 (FIG. 11). The T cell avidity responses elicited were better when the IL-13Rα2 was expressed in the priming vector rather than the boost. All groups of animals showed superior T cell avidity responses compared to mice receiving vectors not containing IL-13Rα2.

### Cytoline Expression

Cytokines and chemokine production are an important aspect of protective immunity. The capacity of CD8+T cells from mice immunised with control vaccines or those expressing IL-13Rα2 was measured at 14 days using an antibody array assay (RayBio Mouse Cytokine Antibody Array). FIG. 12 illustrates that vaccines encoding IL-13Rα2 are superior at eliciting responses that produce higher levels of a range of cytokines and chemokines.

Vaccines that elicit long-term memory would have significant advantages for protection against infection. FIGS. 13A-C show that vaccines encoding IL-13Rα2 elicit enhanced CD8+ T cell responses at 8 weeks post vaccination compared to control vaccines as measured by tetramer binding (FIG. 13A), ICS (FIG. 13B) and IFN-γ ELIspot (FIG. 13C).

As a measure of protective immunity prime boost immunised mice were challenged with an influenza virus encoding the dominant HIV K^{d}Gag₁₉₇₋₂₀₅ epitope. The weight loss observed reflects the capacity of vaccinated mice to resist influenza HIV K^{d}Gag₁₉₇₋₂₀₅ challenge. Mice vaccinated with vectors encoding IL-13Rα2 were better protected against challenge than mice receiving control vaccines and mice not vaccinated (FIG. 14)

In order to demonstrate that IL-13Rα2 needs to be co expressed by the vaccine vectors normal BALB/c mice were immunised with control vaccines and given recombinant IL-13Rα2 separately. FIG. 15 shows that the recombinant IL-13Rα2 protein given separately with control vaccines failed to elicit the responses obtained with vaccines encoding and co expressing IL-13Rα2.

Administration of FPV gag/pol IL-4C118 and VV gag/pol IL-4C118 in a prime-boost vaccination regime was compared with FPV gag/pol and VV gag/pol. Results indicate that the vaccination regime including IL4C118 can enhance the K^{d}Gag₁₉₇₋₂₀₅ specific T-cell avidity as demonstrated by tetramer dissociation (FIG. 16A) as well as the magnitude of the T-cell immunity as demonstrated by the number of CD8+/IFNγ+ positive cells (FIG. 16B) compared to the control FPV-HIVNV-HIV prime boost immunisation.

### Discussion

The inventors had hypothesised that vaccines engineered to express the soluble decoy receptor to IL-13 (IL-13Rα2) would elicit high avidity T cell responses and improve the efficacy of the vaccine-elicited immunity.

Using a prime boost immunisation protocol as shown above, it was shown that recombinant FPV and VV expressing the IL-13Rα2 decoy receptor induce high avidity CD8 T cell responses to co-expressed HIV antigens.

Further, the CD8 T cells induced were polyfunctional in that they expressed multiple cytokines including IFN-γ, TNF-α and IL-2 and are highly protective against a mucosal model challenge of influenza virus expressing a major dominant HIV epitope.

Accordingly, it is believed that such vaccines will be useful in providing improved immunity to the recipient as they inhibit the development of detrimental Th2 host responses.

The disclosure of every patent, patent application, and publication cited herein is hereby incorporated herein by reference in its entirety.

The citation of any reference herein should not be construed as an admission that such reference is available as “Prior Art” to the instant application.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1. An immunomodulatory composition comprising a first agent comprising an immune stimulator that stimulates or otherwise enhances an immune response to the target antigen in a subject or a polynucleotide sequence encoding an immune stimulator that stimulates or otherwise enhances an immune response to the target antigen in a subject together with a second agent comprising an inhibitor of IL-13 function or a polynucleotide sequence encoding an inhibitor of IL-13 function.

2. The composition of claim 1, wherein the composition comprises a nucleic acid composition comprising: a first agent comprising a first polynucleotide sequence which encodes an immune stimulator that stimulates or otherwise enhances an immune response to the target antigen and which is operably linked to a regulatory polynucleotide, and a second agent comprising a second polynucleotide sequence which encodes an inhibitor of IL-13 function and which is operably linked to a regulatory polynucleotide.

3. The composition of claim 1, wherein the first agent and the second agent are in the form of a single composition.

4. The composition of claim 2, wherein the first agent and the second agent are in the form of one or more nucleic acid constructs.

5. The composition of claim 1, wherein the composition is formulated such that the first agent and the second agent are co-expressed in the subject.

6. A method for stimulating an immune response against a target antigen in a subject, the method comprising administering the composition of claim 1.

7. (canceled)

8. (canceled)

9. An immunomodulatory antigen-presenting cell or antigen-presenting cell precursor which presents an antigen that corresponds to at least a portion of the target antigen, and which expresses or otherwise produces an inhibitor of IL-13 function.

10. A method for producing an immunomodulatory antigen-presenting cell or antigen-presenting cell precursor, the method comprising contacting an antigen-presenting cell or antigen-presenting cell precursor with (1) an antigen that corresponds to at least a portion of the target antigen or a polynucleotide from which the antigen is expressible for a time and under conditions sufficient for the antigen or a processed form thereof to be presented by the antigen-presenting cell or antigen-presenting cell precursor, and (2) with a composition according to claim 1, for a time and under conditions for the inhibitor of IL-13 function to be produced by or otherwise provided in the antigen-presenting cell or antigen-presenting cell precursor.

11. A method according to claim 10, wherein the inhibitor of IL-13 function is secreted by the antigen-presenting cell or antigen-presenting cell precursor.

12. The method of claim 6, wherein the composition comprises a nucleic acid composition comprising: a first agent comprising a first polynucleotide sequence which encodes an immune stimulator that stimulates or otherwise enhances an immune response to the target antigen and which is operably linked to a regulatory polynucleotide, and a second agent comprising a second polynucleotide sequence which encodes an inhibitor of IL-13 function and which is operably linked to a regulatory polynucleotide.

13. The method of claim 6, wherein the first agent and the second agent are in the form of a single composition.

14. The method of claim 6, wherein the first agent and the second agent are in the form of one or more nucleic acid constructs.

15. The method of claim 6, wherein the composition is formulated such that the first agent and the second agent are co-expressed in the subject.

16. The method of claim 6, further comprising preventing or treating a disease or condition associated with the presence or aberrant expression of the target antigen in a subject.
